SEQUENCE LISTING (24 sequences)

SEQ ID NO: 1 - DNA, 386 bp, Human Adenovirus
atggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtcttcccccgggctattcggtcgctttttagcactgaccgatgtgaatcaacctgatgtgtttaccgagtcttacattatgcatccggacatgaccgaggagctgtcagtggtgctttttaatcacggtgaccagtttttttacggtcacgccggcatggccgtagtccgtcttatgcttataagggttgtttttcctgttgtaagacaggcttctaatgtttaa

SEQ ID NO: 2 - DNA, 381 bp, Human Adenovirus
atggctgatcaacatatatatgtgcatttgctgggacgtcgggcctttttgccccagcaacagggttattctaatatgtatgttcttttttcaccggaggattttgtgcttgctcccagagggattattttgctgtctttgcagctgtcgttggatattcccactgggtatctgggacgttttttttctgtggcggatatgaatgtgaggggggtattgctgtgtgctcaggagatccaaccgagtacatggtgggaagtttctgttgttctttttaatcattcggatgaattttaccgcggttcccggggacagccggttgcttgcctgctgctggagcgtgtcatatatcccaccgttcgccaagcttctttagtttaa

SEQ ID NO: 3 - protein, 126 aa, Human Adenovirus
MADQHIYVHLLGRRAFLPQQQGYSNMYVLFSPEDFVLAPRGIILLSLQLSLDIPTGYLGRFFSVADMNVRGVLLCAQEIQPSTWWEVSVVLFNHSDEFYRGSRGQPVACLLLERVIYPTVRQASLV

SEQ ID NO: 4 - DNA, 387 bp, Human Adenovirus
atggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaa

SEQ ID NO: 5 - protein, 128 aa, Human Adenovirus
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 6 - DNA, 387 bp, Human Adenovirus
atggctgccgctgtggaagcgctgtttgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaa

SEQ ID NO: 7 - protein, 128 aa, Human Adenovirus
MAAAVEALFVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 8 - DNA, 411 bp, Human Adenovirus
atggactacaaagacgatgacgacaaggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaa

SEQ ID NO: 9 - protein, 136 aa, Human Adenovirus
MDYKDDDDKAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 10 - protein, 9 aa, Artificial Sequence
  A synthetic sequence
MDYKDDDDK

SEQ ID NO: 11 - DNA, 411 bp, Human Adenovirus
atggactacaaagacgatgacgacaaggctgccgctgtggaagcgctgtttgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaa

SEQ ID NO: 12 - protein, 136 aa, Human Adenovirus
MDYKDDDDKAAAVEALFVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 13 - DNA, 381 bp, Human Adenovirus; identical to SEQ ID NO: 2

SEQ ID NO: 14 - protein, 126 aa, Human Adenovirus; identical to SEQ ID NO: 3

SEQ ID NO: 15 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 16 - DNA, 443 bp, Homo sapiens
atggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcacta

SEQ ID NO: 17 - protein, 352 aa, Homo sapiens [sequence omitted]

SEQ ID NO: 18 - DNA, 3358 bp, Homo sapiens
agaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataa

SEQ ID NO: 19 - protein, 1455 aa, Homo sapiens
MSDSWVPNSASGQDPGGRRRAWAELLAGRVKREKYNPERAQKLKESAVRLLRSHQDLNALLLEVEGPLCKKLSLSKVIDCDSSEAYANHSSSFIGSALQDQASRLGVPVGILSAGMVASSVGQICTAPAETSHPVLLTVEQRKKLSSLLEFAQYLLAHSMFSRLSFCQELWKIQSSLLLEAVWHLHVQGIVSLQELLESHPDMHAVGSWLFRNLCCLCEQMEASCQHADVARAMLSDFVQMFVLRGFQKNSDLRRTVEPEKMPQVTVDVLQRMLIFALDALAAGVQEESSTHKIVRCWFGVFSGHTLGSVISTDPLKRFFSHTLTQILTHSPVLKASDAVQMQREWSFARTHPLLTSLYRRLFVMLSAEELVGHLQEVLETQEVHWQRVLSFVSALVVCFPEAQQLLEDWVARLMAQAFESCQLDSMVTAFLVVRQAALEGPSAFLSYADWFKASFGSTRGYHGCSKKALVFLFTFLSELVPFESPRYLQVHILHPPLVPGKYRSLLTDYISLAKTRLADLKVSIENMGLYEDLSSAGDITEPHSQALQDVEKAIMVFEHTGNIPVTVMEASIFRRPYYVSHFLPALLTPRVLPKVPDSRVAFIESLKRADKIPPSLYSTYCQACSAAEEKPEDAALGVRAEPNSAEEPLGQLTAALGELRASMTDPSQRDVISAQVAVISERLRAVLGHNEDDSSVEISKIQLSINTPRLEPREHMAVDLLLTSFCQNLMAASSVAPPERQGPWAALFVRTMCGRVLPAVLTRLCQLLRHQGPSLSAPHVLGLAALAVHLGESRSALPEVDVGPPAPGAGLPVPALFDSLLTCRTRDSLFFCLKFCTAAISYSLCKFSSQSRDTLCSCLSPGLIKKFQFLMFRLFSEARQPLSEEDVASLSWRPLHLPSADWQRAALSLWTHRTFREVLKEEDVHLTYQDWLHLELEIQPEADALSDTERQDFHQWAIHEHFLPESSASGGCDGDLQAACTILVNALMDFHQSSRSYDHSENSDLVFGGRTGNEDIISRLQEMVADLELQQDLIVPLGHTPSQEHFLFEIFRRRLQALTSGWSVAASLQRQRELLMYKRILLRLPSSVLCGSSFQAEQPITARCEQFFHLVNSEMRNFCSHGGALTQDITAHFFRGLLNACLRSRDPSLMVDFILAKCQTKCPLILTSALVWWPSLEPVLLCRWRRHCQSPLPRELQKLQEGRQFASDFLSPEAASPAPNPDWLSAAALHFAIQQVREENIRKQLKKLDCEREELLVFLFFFSLMGLLSSHLTSNSTTDLPKAFHVCAAILECLEKRKISWLALFQLTESDLRLGRLLLRVAPDQHTRLLPFAFYSLLSYFHEDAAIREEAFLHVAVDMYLKLVQLFVAGDTSTVSPPAGRSLELKGQGNPVELITKARLFLLQLIPRCPKKSFSHVAELLADRGDCDPEVSAALQSRQQAAPDADLSQEPHLF

SEQ ID NO: 20 - DNA, 5452 bp, Homo sapiens
gagccgccgccggggctgtaggcgccaaggccatgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccggcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacatggctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctgacgggacctgccactgcacaccagcccagctcccgtgtaaataatttattacaagcataacatggagctcttgttgcactaaaaagtggattacaaatctcctcgactgctttagtggggaaaggaatcaattatttatgaactgtccggccccgagtcactcagcgtttgcgggaaaataaaccactggtcccagagcagaggaaggctacttgagccggacaccaagcccgcctccagcaccaagggcgggcagcaccctccgaccctcccatgcgggtgcacacgaagggtgaggctgacacagccactgcggagtccaggctgctagaggtgctcatcctcactgccgtcctcaggtgggttcgggcttcaccgcctggccctctgtggtcacagaggggctcggtggcccaggtggtggttccgcctccaggggcagggccttgtcctgggtctgtgtcagcgggtgcaccatggacatgtgtacattgaggttgtgggccttctcaaaccgccggccacactggtcacaggcaaagtccagctcagtctcagccttgtgtttggtcatgtggtacttgagggatgcccgctgcctgcactggaacccacagacctcacacctgggggacagaggcagataagaaggtgcgagggccacagccctgggagggggtcctgactcacacttactgcaaaggcttggctcccgaatgtcgcatttggtggacgagaaggtgcttccgctgcttgaaggtttgtccacattcgtcacagatatagttccgcacctctgagaggggagagtccagtgagtccaggcccctgatgctccaacctcccggggggacgacgatgacaatgtgaaaccatcacagctgggaagacatttctgcacatggttcaccatgcagtgggcccaagcaaggggcctatgagggcctcgtttattaagatctttaaactgctttatacactgtcacgtggcttcatcagctgtgtgcatttcaggatggtttttaaagaaacctcagaaagctatttccttaa

SEQ ID NO: 21 - protein, 558 aa, Homo sapiens
MAQDSVDLSCDYQFWMQKLSVWDQASTLETQQDTCLHVAQFQEFLRKMYEALKEMDSNTVIERFPTIGQLLAKACWNPFILAYDESQKILIWCLCCLINKEPQNSGQSKLNSWIQGVLSHILSALRFDKEVALFTQGLGYAPIDYYPGLLKNMVLSLASELRENHLNGFNTQRRMAPERVASLSRVCVPLITLTDVDPLVEALLICHGREPQEILQPEFFEAVNEAILLKKISLPMSAVVCLWLRHLPSLEKAMLHLFEKLISSERNCLRRIECFIKDSSLPQAACHPAIFRVVDEMFRCALLETDGALEIIATIQVFTQCFVEALEKASKQLRFALKTYFPYTSPSLAMVLLQDPQDIPRGHWLQTLKHISELLREAVEDQTHGSCGGPFESWFLFIHFGGWAEMVAEQLLMSAAEPPTALLWLLAFYYGPRDGRQQRAQTMVQVKAVLGHLLAMSRSSSLSAQDLQTVAGQGTDTDLRAPAQQLIRHLLLNFLLWAPGGHTIAWDVITLMAHTAEITHEIIGFLDQTLYRWNRLGIESPRSEKLARELLKELRTQV

SEQ ID NO: 22 - DNA, 4592 bp, Homo sapiens
agaatgcactgctgacacgtgtgcgcgcgcgcggctccactgccgggcgaccgcgggaaaattccaaaaaaactcaaaaagccaatacgaggcaaagccaaattttcaagccacagatcccgggcggtggcttcctttccgccactgcccaaactgctgaagcagctcccgcgaggaccacccgatttaatgtgtgccgaccatttccttcagtgctggacaggctgctgtgaagggacatcaccttttcgctttttccaagatggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatggtccaggtgaaggccgtgctgggccacctcctggcaatgtccagaagcagcagcctctcagcccaggacctgcagacggtagcaggacagggcacagacacagacctcagagctcctgcacaacagctgatcaggcaccttctcctcaacttcctgctctgggctcctggaggccacacgatcgcctgggatgtcatcaccctgatggctcacactgctgagataactcacgagatcattggctttcttgaccagaccttgtacagatggaatcgtcttggcattgaaagccctagatcagaaaaactggcccgagagctccttaaagagctgcgaactcaagtctagaaggcacgcaggccgtgtgggtgcccggcgtgagggatcaggctcgccagggccacaggacaggtgatgacctgtggccacgcatttgtggagtaagtgccctcgctgggctgtgagaatgagctgtacacatcttgggacaatctgctagtatctattttacaaaatgcagagccaggtccctcagcccagactcagtcagacatgttcactaatgactcaagtgagccttcggtactcctggtgcccgcccggccagaccgtcagcttgataattactaaagcaaaggcctgggtgggagaacaggtttctagtttttacccaagtcaagctgcacatctattatttaaaaattcaaagtcttagaaccaagaatttggtcatgaaccattaaagaatttagagagaacttagctctttttagactctttttaggagtcagggatctgggataaagccacactgtcttgctgtatggagaaattcttcaaggggagtcagggtccctcaggcttcccttgtgtctccctggacctgcctgacaggccacaggagcagacagcacacccaagcccgggcctccggcacactctttccactctgtatttgctaaatgatgctaactgctaccaaaaggcccttgggacatcagaggagccggcaggcgaaggtagaggatgtgttccagaaacattagaaggcaggattaattcagttagttagttctcttgttaaatggaaatgggaattggaaattcctgataaagaattggcctggctgggtgcagtggctcacacctgtgatcccagcactttgggaggccaaggcagggggattacttcagcccaggagttccagactgcctggctaacatggcaataccctatctctactaaaaatacaaaaattatcggggtgcaatggcatgcatctgtaatcccagctattcaagaggctgaggcatgaggatctcttgaacccgggaggtgggagttgtagtgagccgagatcatgacactgcactccagcctgggcaacagagcgagaccatctcttaaaaaaaggcattgttagtgtaatctcaaggttaacatttatttcatgtcagtacagggtgctttttcctttcagggacattctggaattgtattggttgtacattcttttgtgtctattctgtttgtcaagtgagtcaagacttgcttttgtccattttgatttgtgtgtattagtctgagtcttggctccgttttgaggtatgagcaaagttttgctggattagaagttaacctttagggaaattccttattttggtatgtggcaatgctaatagatccactgaagatctggaaaattccaggaacttttcacctgagcctttcttctgagaaatgctgcagtcagaagggtgtgctggtaaagtattttggtggcagctgccatcatggtcattgccttcatataacatgcttcgtgctcatggtcattgccttcatataacatgcttcgtgccatcatgatccttgccttcatataacaaacatgcttcgtcagaggtgttggggttgaaaaaggagctgcatgcttcactggagttgagggcctctctcctgttctgactttaagccagaacttgtggctgggccatggaagctgtgactcctctgtggacatggtggcagcagggaacccctagagagaggggccactgggaccaggcctcctgttgtggagggactcctgggacagtcctccaccctgtcctgtggtcctgtgtacagggttggcctcttcctcctcccctgccaggcctctgcccatgccccttccttccttctcctgggactggtgaagctaggcatctggaagacttcttcctagcctggaagccctgacctcggcccatctgcagaatctcccagttccttcacagctgccgagtcctctcacgggtgcggtggaggcggccttgccggtggtgctttctgggcagccaggggttcctgggtgggaggactgtccctctggggacgtggcactgaagtgcctgctggcttcatgtggccctttgccctttcccagcctgagagatgctcaaaggtggggagctgggggagccacccctcggccattccctccacctccaagacaggtggcggccgggcaggcactcttaagcccacctccccctcttgttgccttcgatttcggcaaagcctgggcaggtgccaccgggaaggaatggcatccgagatgctgggcggggacgcggcgtggccgagggggccttgacggcgttggcggggcctgggcacaggggcagccgcagggaggcagggatggcaaggcgtgaagccaccctggaaggaactggaccaaggtcttcagaggtgcgacagggtctggaatctgaccttactctagcaggagtttttgtagactctccctgatagtttagtttttgataaagcatgctggtaaaaccactaccctcagagagagccaaaaatacagaagaggcggagagcgcccctccaaccaggctgttattcccctggactccgtgacatctgtggaattttttagctctttaaaatctgtaatttgttgtctattttttcattctaaataaaacttcagtttgcacctaa

SEQ ID NO: 23 - protein, 622 aa, Homo sapiens
MSRQTTSVGSSCLDLWREKNDRLVRQAKVAQNSGLTLRRQQLAQDALEGLRGLLHSLQGLPAAVPVLPLELTVTCNFIILRASLAQGFTEDQAQDIQRSLERVLETQEQQGPRLEQGLRELWDSVLRASCLLPELLSALHRLVGLQAALWLSADRLGDLALLLETLNGSQSGASKDLLLLLKTWSPPAEELDAPLTLQDAQGLKDVLLTAFAYRQGLQELITGNPDKALSSLHEAASGLCPRPVLVQVYTALGSCHRKMGNPQRALLYLVAALKEGSAWGPPLLEASRLYQQLGDTTAELESLELLVEALNVPCSSKAPQFLIEVELLLPPPDLASPLHCGTQSQTKHILASRCLQTGRAGDAAEHYLDLLALLLDSSEPRFSPPPSPPGPCMPEVFLEAAVALIQAGRAQDALTLCEELLSRTSSLLPKMSRLWEDARKGTKELPYCPLWVSATHLLQGQAWVQLGAQKVAISEFSRCLELLFRATPEEKEQGAAFNCEQGCKSDAALQQLRAAALISRGLEWVASGQDTKALQDFLLSVQMCPGNRDTYFHLLQTLKRLDRRDEATALWWRLEAQTKGSHEDALWSLPLYLESYLSWIRPSDRDAFLEEFRTSLPKSCDL

SEQ ID NO: 24 - DNA, 2556 bp, Homo sapiens
cctttctcgaggctgtggcctccgcgagagccgagcgggccgcaccgccggccgtgcgactgccccagtcagacacgaccccggcttctagcccgcctaagcctgtttggggttgctgactcgtttcctccccgagtttcccgcgggaactaactcttcaagaggaccaaccgcagcccagagcttcgcagacccggccaaccagaggcgaggttgagagcccggcgggccgcggggagagagcgtcccatctgtcctggaaagcctgggcgggtggattgggaccccgagagaagcaggggagctcggcggggtgcagaagtgcccaggcccctccccgctggggttgggagcttgggcaggccagcttcacccttcctaagtccgcttctggtctccgggcccagcctcggccaccatgtcccgccagaccacctctgtgggctccagctgcctggacctgtggagggaaaagaatgaccggctcgttcgacaggccaaggtggctcagaactccggtctgactctgaggcgacagcagttggctcaggatgcactggaagggctcagagggctcctccatagtctgcaagggctccctgcagctgttcctgttcttcccttggagctgactgtcacctgcaacttcattatcctgagggcaagcttggcccagggtttcacagaggatcaggcccaggatatccagcggagcctagagagagtgctggagacacaggagcagcaggggcccaggttggaacaggggctcagggagctgtgggactctgtccttcgtgcttcctgccttctgccggagctgctgtctgccctgcaccgcctggttggcctgcaggctgccctctggttgagtgctgaccgtcttggggacctggccttgttactagagaccctgaatggcagccagagtggagcctctaaggatctgctgttacttctgaaaacttggagtcccccagctgaggaattagatgctccattgaccctgcaggatgcccagggattgaaggatgtcctcctgacagcatttgcctaccgccaaggtctccaggagctgatcacagggaacccagacaaggcactaagcagccttcatgaagcggcctcaggcctgtgtccacggcctgtgttggtccaggtgtacacagcactggggtcctgtcaccgtaagatgggaaatccacagagagcactgttgtacttggttgcagccctgaaagagggatcagcctggggtcctccacttctggaggcctctaggctctatcagcaactgggggacacaacagcagagctggagagtctggagctgctagttgaggccttgaatgtcccatgcagttccaaagccccgcagtttctcattgaggtagaattactactgccaccacctgacctagcctcaccccttcattgtggcactcagagccagaccaagcacatactagcaagcaggtgcctacagacggggagggcaggagacgctgcagagcattacttggacctgctggccctgttgctggatagctcggagccaaggttctccccacccccctcccctccagggccctgtatgcctgaggtgtttttggaggcagcggtagcactgatccaggcaggcagagcccaagatgccttgactctatgtgaggagttgctcagccgcacatcatctctgctacccaagatgtcccggctgtgggaagatgccagaaaaggaaccaaggaactgccatactgcccactctgggtctctgccacccacctgcttcagggccaggcctgggttcaactgggtgcccaaaaagtggcaattagtgaatttagcaggtgcctcgagctgctcttccgggccacacctgaggaaaaagaacaaggggcagctttcaactgtgagcagggatgtaagtcagatgcggcactgcagcagcttcgggcagccgccctaattagtcgtggactggaatgggtagccagcggccaggataccaaagccttacaggacttcctcctcagtgtgcagatgtgcccaggtaatcgagacacttactttcacctgcttcagactctgaagaggctagatcggagggatgaggccactgcactctggtggaggctggaggcccaaactaaggggtcacatgaagatgctctgtggtctctccccctgtacctagaaagctatttgagctggatccgtccctctgatcgtgacgccttccttgaagaatttcggacatctctgccaaagtcttgtgacctgtagctgccacgttttgaagagcttgagctgggtccccagtgggctgtctctctgtggggagggctttctgcttcaccatcattaggaatgtgaccattcctatataattcctggactggtgagattggtggtaggcctgtgaaatttgccctagttactaccattctcgttttggaggaaacaatctctgccaccaccaagtcattgactttgctcgaggcaccttttttcctgtttctccttttctgttgtcgagtaaaatttcatattta

(19) United States
(12) Patent Application Publication
(10) Pub. No.: US 2023/0212516 A1
(43) Pub. Date: Jul. 6, 2023

(54) METHODS FOR EXPANDING HEMATOPOIETIC STEM CELLS

(71) Applicant: Cornell University, Ithaca, NY (US)

(72) Inventors: Joseph M. Scandura, New York, NY (US); Silvana Di Giandomenico, New York, NY (US)

(21) Appl. No.: 18/000,603

(22) PCT Filed: Jun. 2, 2021
(86) PCT No.: PCT/US2021/035511

§ 371 Date: Dec. 2, 2022

Related U.S. Application Data
(60) Provisional application No. 63/033,453, filed on Jun. 2, 2020.

(51) Int. Cl. C12N 5/0789

(52) U.S. Cl. CPC C12N 5/0647; C12N 2500/02; C12N 2502/28; C12N 2510/00

(57) ABSTRACT

Methods and compositions are described herein that involve expansion of hematopoietic stem cells and/or hematopoietic progenitor cells.

### CROSS-REFERENCE TO RELATED APPLICATIONS

This application claims the benefit of U.S. Provisional Patent Appl. Ser. No. 63/033,453, filed Jun. 2, 2020, which is incorporated by reference as if fully set forth herein.

### INCORPORATION BY REFERENCE OF SEQUENCE LISTING PROVIDED AS A TEXT FILE

A Sequence Listing is provided herewith as a text file, “2146358.txt” created on Jun. 2, 2021 and having a size of 57,344 bytes. The contents of the text file are incorporated by reference herein in their entirety.

### BACKGROUND

Hematopoietic stem cells (HSCs) promote the lifelong production of all mature blood cell lineages through their unique capabilities of durable self-renewal and multilineage differentiation. HSCs are present in donor-derived bone marrow (BM), cord blood (CB), and mobilized peripheral blood stem-cell products for allogeneic stem-cell transplantation (SCT) in patients with hematological malignancies and monogenic diseases. Patient-derived peripheral blood stem-cell products are extensively used for autologous SCT, which supports hematopoietic rescue after high-dose chemotherapy for various types of hematological malignancies, solid tumors, and autoimmune diseases. However, in many diseases affecting blood cell lineages, the number of HSCs that can be obtained from a patient is very limited in number. Attrition of autologous HSCs during processing for gene therapy is also a problem. In addition, it has not been possible to maintain or expand human HSCs ex vivo. Under in vitro cell culture conditions, the number of HCS decline because HSCs either die or terminally differentiate, losing their stem cell properties.

### SUMMARY

The present disclosure is directed to methods of culturing and expanding HSCs in vitro. In some embodiments, HSCs cultured according to the methods of the instant disclosure expand (increase in number) at least 3 fold, at least 5 fold, at least 10 fold, at least 15 fold, at least 20 fold, at least 25 fold, at least 30 fold, at least 35 fold, at least 40 fold, at least 45 fold, or at least 50 fold.

The inventors of the instant disclosure have found that culturing HSCs under hypoxic conditions promotes HSC self-renewal, and that culturing HSCs under normoxic (for example, ambient oxygen) conditions is detrimental to HSC survival and self-renewal. Without being bound to a particular theory, hypoxic conditions are thought to reduce reactive oxygen species (ROS), induce HIF1α expression, and stimulate HIF1α-related signaling pathways in the cells, thereby promoting HSC self-renewal.

As used herein, the phrase “hypoxic conditions” refers to culture conditions of 3% or less oxygen. In some embodiments. “hypoxic conditions” refer to about 2.5%, about 2%, about 1% or less oxygen. As used herein, the phrase “normoxic conditions” refer to culture conditions of more than 3% oxygen. In some embodiments, “normoxic conditions” refer to about 5%, about 8%, about 10%, about 15%, or ambient/atmospheric conditions of about 20% oxygen. As used herein reactive oxygen species (ROS) are highly reactive chemical molecules formed due to the electron receptivity of O₂. Examples of ROS include peroxides, superoxide, hydroxyl radical, singlet oxygen, and alpha-oxygen.

The inventors have also found that culturing HSCs in the presence of endothelial feeder cells (ECs) improves HSC survival and self-renewal capabilities. In some embodiments, the HSC-endothelial feeder cell co-culture is done under hypoxic conditions.

In one aspect, the disclosure is directed to a method of expanding HSCs comprising co-culturing the HSCs with feeder endothelial cells (ECs) under hypoxic conditions, wherein the ECs do not express the adenovirus E4 open reading frame 1 (E4ORF1) gene, and the co-culturing is performed in a medium that comprises a cytokine. In some embodiments, the cytokine is selected from the group consisting of FGF2, IGF, EGF, and VEGF. In a specific embodiment, the cytokine is FGF2. In some embodiments, the medium further comprises heparin.

In another aspect, the disclosure is directed to a method of expanding HSCs comprising co-culturing the HSCs with feeder endothelial cells (ECs) under hypoxic conditions, wherein the ECs are transfected to express either the adenovirus E4 open reading frame 1 (E4ORF1) gene, or an Akt gene.

In another aspect, the disclosure is directed to a method of expanding HSCs comprising co-culturing the HSCs with feeder endothelial cells (ECs) under normoxic conditions (for example, ambient/atmospheric conditions of 18%-20% oxygen), wherein the co-culturing is performed in a medium that comprises a factor selected from the group consisting of a TGFβ signaling inhibitor, a TGFβ inhibitor, a chemokine, a hypoxia mimetic, a ROS scavenger, a HIF stabilizer, and an mTOR inhibitor. In some embodiments, the ECs do not express the adenovirus E4 open reading frame 1 (E4ORF1) gene, and the co-culturing is performed in a medium that comprises a cytokine. In some embodiments, the ECs are transfected to express either the adenovirus E4 open reading frame 1 (E4ORF1) gene, or the Akt gene.

In another aspect, the disclosure is directed to a method of expanding HSCs comprising culturing the HSCs in the absence of feeder endothelial cells (ECs) under normoxic conditions, wherein the co-culturing is performed in a medium that comprises a factor selected from the group consisting of a TGFβ signaling inhibitor, a chemokine, a hypoxia mimetic, a ROS scavenger, a HIF stabilizer, and an mTOR inhibitor.

As mentioned above, HSCs are very difficult to maintain ex vivo, because they quickly die or differentiate under normal culture conditions. The inventors of the instant disclosure have found conditions that support HSC viability, which allows for in vitro manipulation (e.g., genetic manipulation) and expansion of the HSCs. Therefore, another aspect of the disclosure is directed to a method for maintaining HCSs in vitro comprising culturing the HSCs under hypoxic conditions. Yet another aspect of the disclosure is directed to a method of maintaining hematopoietic stem cells (HSCs) in vitro comprising culturing the HSCs under normoxic conditions (for example, ambient/atmospheric conditions of 18%-20% oxygen) and in a culturing medium that comprises a factor selected from the group consisting of a hypoxia mimetic, a ROS scavenger, and a HIF stabilizer.

In another aspect, the disclosure is directed to a method of treating subject suffering from a disease comprising a genetic defect affecting hematopoietic cells comprising harvesting hematopoietic stem cells (HSCs) from the subject, culturing the HSCs according to the methods of this disclosure; genetically manipulating the expanded HSCs to correct the genetic defect; and administering the genetically-manipulated HSC to the subject.

In some embodiments, the HSCs are obtained from the bone marrow or blood (such as umbilical cord blood or adult peripheral blood) of a human subject. In some embodiments, the phrase “adult HSC” refers to HSC obtained from a source other than umbilical cord blood (UCB).

In some embodiments, the human subject suffers from a disease caused by a genetic defect that affects hematopoietic cells, for example, Fanconi Anemia (FA), sickle cell anemia, severe combined immunodeficiency, β-thalassemia. Wiskott-Aldrich syndrome, adenosine deaminase SCID (ADA SCID), HIV, Diamond-Blackfan anemia, Schwachman-Diamond syndrome metachromatic leukodystrophy, and leukodystrophy. Other genes and genetic variations can also be modified such as genes involved in metabolism or genes involved in drug responses. In some embodiments, the human subject suffers from a disease for which genetic modification of normal genes (e.g., CCR5 in HIV infection) could cure or significantly ameliorate the disease.

“TGFβ inhibitors” or “TGFβ signaling inhibitors” as used herein, refer to molecules which inhibit the signal transduction mediated by TGFβ. TGFβ signaling inhibitors include molecules which inhibit the level and/or activity of TGFβ such as agents that block the upstream synthesis and activation of latent TGFβ to form active TGFβ; agents that prevent the release/secretion of latent or active TGFβ from megakaryocytes; agents that block the interaction between TGFβ with its receptors, and agents that inhibit the downstream signaling cascade, such as molecules which inhibit the level and/or activity of downstream targets of TGFβ signaling, for example, p57, among others (including those in the Smad-dependent pathway of TGFβ signaling). TGFβ-pathway inhibitors also include molecules that inhibit the function or activity of TGFβ receptors. See, e.g., Nagaraj and Datta, Exp. Opin. Investig. Drugs 19(1): 77-91 (2010); Korpal and Yang. Eur J Cancer 46: 1232-1240 (2010); and Akhurst et al., Nature Reviews 11:791 (2012); all of which are incorporated herein by reference in entirety. The TGFβ inhibitors reduce the expression or function of TGFβ1, TGFβ2, TGFβ3, or a combination thereof.

TGFβ inhibitors suitable for use in the present methods include large molecule inhibitors (such as monoclonal antibodies, and soluble TGFβ antagonists such as polypeptides composed of the extracellular domain of a TGFβ receptor), antisense oligonucleotides, and small molecule organic compounds. For example, the TGFβ inhibitor can be an anti-TGFβ-1,2,3 monoclonal antibody (e.g., 1D11.16.8 from Invitrogen or Thermofisher Scientific).

### DESCRIPTION OF THE FIGURES

FIG. 1A-1R illustrate methods and results for achieving expansion of functional Adult Derived-human hematopoietic stem and progenitor cells (AD-HSPCs) by hypoxic endothelial cells. FIG. 1A is a schematic diagram illustrating methods for ex vivo expansion of AD-HSPCs with or without endothelial cells. Fold change in cell numbers after expansion was calculated relative to the cell numbers originally present for all populations of interest. Unexpanded cells from the same donor (UN) were used in the experiments. As shown, the bottom wells have endothelial feeder cells (EC) but the top wells do not (noEC). FIG. 1B graphically illustrates the fold change in CD45+, CD45+CD34+(CD34+), and immunophenotypically-defined HSCs (iHSC) (CD45+CD34+CD45RA-CD90+) cells cultured with E4-expressing endothelial cells (EC) or without EC (NoEC). FIG. 1C graphically illustrates the fold change of colony forming cells (CFU) for granulocyte-macrophage cells (CFU-GM; GM), for burst-forming unit-erythroid (BFU-E) cells (CFU-BFU-E; BFU-E), and for colony forming units that generate myeloid cells (CFU-GEMM; GEMM) (n=4 donors per condition). FIG. 1D is a schematic diagram illustrating methods for testing cells before transplantation into immunodeficient (NSG) mice. Engraftment of unexpanded cells from the same donor was used as control (UN). Engraftment of the equivalent number of cells was assessed after expansion with EC (EC) or without EC (NoEC). FIG. 1E graphically illustrates the percent hCD45 engraftment for unexpanded cells (UN) compared to cells after expansion with EC (EC) or without EC (NoEC). The percent hCD45 engraftment of negative control cells (B6) is also shown (n=5 donors per group). The table below shows the number of transplanted mice meeting criteria for engraftment. FIG. 1F graphically illustrates the fold change of CD45+CD34+ and CD45+CD34− cells when cultured under different oxygen tension conditions (% oxygen) (n=4 donors per condition). FIG. 1G graphically illustrates the fold change of CD45+ cells when cultured under different oxygen tension conditions (% oxygen) (n=4 donors per condition). FIG. 1H graphically illustrates the fold change of CD34+ cells when cultured under different oxygen tension conditions (% oxygen) (n=4 donors per condition). FIG. 1I graphically illustrates the fold change of iHSC (CD34+CD38-CD45RA-CD90+) cells when cultured under different oxygen tension conditions (% oxygen) (n=4 donors per condition). FIG. 1J graphically illustrates the fold change of colony forming units that generate myeloid cells (GEMM) when cultured under different oxygen tension conditions (% oxygen) (n=4 donors per condition). FIG. 1K graphically illustrates the expansion of CD45+CD34+ and CD45+CD34− cells when cultured without endothelial feeder cells under different oxygen tension conditions (% oxygen) using cytokines alone (NoEC) (n=5 donors per group). FIG. 1L graphically illustrates the expansion of CD45+ cells when cultured without endothelial feeder cells under different oxygen tension conditions (% oxygen) using cytokines alone (NoEC) (n=5 donors per group). FIG. 1M graphically illustrates the expansion of CD34+ cells when cultured without endothelial feeder cells under different oxygen tension conditions (% oxygen) using cytokines alone (NoEC) (n=5 donors per group). FIG. 1N graphically illustrates the expansion of CD90 cells when cultured without endothelial feeder cells under different oxygen tension conditions (% oxygen) using cytokines alone (NoEC) (n=5 donors per group). FIG. 1O graphically illustrates the fold change of colony forming units that generate myeloid cells (GEMM) when cultured without endothelial feeder cells under different oxygen tension conditions (% oxygen) using cytokines alone (NoEC) (n=5 donors per group). FIG. 1P graphically illustrates the percentage of hCD45 cells from bone marrow engrafted into the blood of NSG mice that that had been expanded using hypoxia either with or without endothelial cells (n=5 donors per group). Engraftment of unexpanded cells from the same donor was used as control (UN). The percent hCD45 engraftment of negative control cells (B6) is also shown. Data are shown as mean±SD (* P<0.05, ** P<0.01, *** P<0.001, or if not shown, the comparison was not significant). FIG. 1Q graphically ea vivo expansion of AD-HSPCs without endothelial feeder cells but when the culture medium contains cytokines with SR1 or UM171. The fold change in cell numbers is shown for CD45+, CD45+CD34+(CD34+), and iHSC (CD45+CD34+CD45RA-CD90+) cells cultured with cytokines alone (NoRx) or with SR1 or UM171 (n=4 donors per condition). FIG. 1R graphically ex vivo expansion of AD-HSPCs without endothelial feeder cells but when the culture medium contains cytokines with SR1 or UM171. The fold change in colony forming cells is shown for CFU-GM (GM), CFU-BFU-E (BFU-E) CFU-GEMM (GEMM) cells cultured with cytokines alone (NoRx) or with SR1 or UM171 (n=4 donors per condition) (n=4 donors per condition).

FIG. 2A-2B illustrate that the hypoxic vascular niche platform expands AD-HSPCs with all the features of bona fide HSCs. FIG. 2A is a schematic illustrating a method used for a limiting dilution experiment. Cell dose was calculated based upon the initial number of unexpanded AD-HSPCs. FIG. 2B graphically illustrates results of limiting-dilution transplantation demonstrating vascular niche 22-fold expansion of GCSF-mobilized AD-HSPCs capable of long-term, multi-lineage NSG engraftment.

FIG. 3A-3O illustrates that decreasing reactive oxygen species (ROS) or stabilizing HIF1α favors ex vivo AD-HSPC Expansion. FIG. 3A is a schematic illustrating ROS measurement using CellROX reagent. AD-HSPCs were cultured without (NoEC) or with EC (EC) at different oxygen tensions. After 4 days culture, cultures were exposed for 30 minutes prior to harvest and analysis. FIG. 3B graphically illustrates CellRox mean fluorescence intensity (MFI) for the indicated hematopoietic populations and the oxygen tension during culture without endothelial feeder cells (NoEC)(n=4 donors per condition). FIG. 3C graphically illustrates CellRox mean fluorescence intensity (MFI) is shown for the indicated hematopoietic populations and the oxygen tension during culture without endothelial feeder cells (EC) (n=4 donors per condition). FIG. 3D graphically illustrates γH2AX mean MFI for the CD45 hematopoietic cell populations cultured at various oxygen levels with endothelial cells (n=3 donors per condition). FIG. 3E graphically illustrates γH2AX mean MFI for the CD34 hematopoietic cell populations cultured at various oxygen levels with endothelial cells (n=3 donors per condition). FIG. 3F graphically illustrates γH2AX mean MFI for the iHSC hematopoietic cell populations cultured at various oxygen levels with endothelial cells (n=3 donors per condition). FIG. 3G graphically illustrates γH2AX mean MFI for the CD31 hematopoietic cell populations cultured at various oxygen levels with endothelial cells (n=3 donors per condition). FIG. 3H is schematic illustrating methods for detecting the effects of N-acetyl cysteine (NAC) during ex vivo propagation of AD-HSPCs using vascular niche platform (EC) at 20% or 1% oxygen. FIG. 3I graphically illustrates the fold change of the indicated immunophenotypic hematopoietic populations (CD45+, CD34+, iHSC) and functions for CFU-GEMM (GEMM) following culture with endothelial cells (ECs) at 20% oxygen, without NAC (NoRx) or with NAC (NAC) (n=4 donors per group). FIG. 3J graphically illustrates the fold change of the indicated immunophenotypic hematopoietic populations (CD45+, CD34+, iHSC) and the functions CFU-GEMM (GEMM) following culture with endothelial cells (ECs) at 1% oxygen, without NAC (NoRx) or with NAC (NAC) (n=4 donors per group). FIG. 3K graphically illustrates the HIF1α mean fluorescence intensity (MFI) for the indicated hematopoietic populations cultured at various oxygen tension levels without endothelial cells (NoEC) (n=3 donors per condition). FMO means Fluorescence Minus One (essentially background fluorescence levels). FIG. 3L graphically illustrates the HIF1α mean fluorescence intensity (MRI) for the indicated hematopoietic populations cultured at various oxygen tension levels with endothelial cells (EC) (n=3 donors per condition). FIG. 3M is a schematic illustrating analysis of an HIF1α stabilizer (IOX2) or an inhibitor of HIF1α (BAY) during ex vivo propagation of AD-HSPCs using vascular niche platform (EC) at 20% or 1% oxygen. FIG. 3N graphically illustrates the fold change of the indicated immunophenotypic hematopoietic populations (CD45+, CD34+, iHSC) and functions of CFU-GEMM (GEMM) following culture with endothelial cells at 20% oxygen, without additive (NoRx) or with IOX2 or BAY (n=3 donors per group). Data are shown as mean±SD (* P<0.05, ** P<0.01, *** P<0.001, or if not shown, the comparison was not significant). FIG. 3O graphically illustrates the fold change of the indicated immunophenotypic hematopoietic populations (CD45+, CD34+, iHSC) and functions CFU-GEMM (GEMM) is shown following culture with ECs at 1% oxygen, without additive (NoRx) or with IOX2 or BAY (n=3 donors per group). Data are shown as mean±SD (* P<0.05, ** P<0.01, *** P<0.001, or if not shown, the comparison was not significant).

FIG. 4A-4J illustrate that hypoxia mediates an angiocrine switch to factors favoring HSC self-renewal. FIG. 4A is a schematic illustrating methods for endothelial cell (EC) culture at different oxygen tensions prior to western blot or RNA-seq. FIG. 4B shows a Western blot and a graph showing quantified phosphorylated and total protein for AKT in ECs cultured at different oxygen tension. FIG. 4C shows a Western blot and a graph showing quantified phosphorylated and total protein for p38 in ECs cultured at different oxygen tension. FIG. 4D shows a Western blot and a graph showing quantified phosphorylated and total protein for pERK in ECs cultured at different oxygen tension. Replicate blots for FIGS. 4B-4D were quantified using ImageJ and phosphorylated forms were normalized to signal for the total corresponding protein (n=3 donors per condition). FIG. 4E graphically illustrates the fold change of the indicated immunophenotypic hematopoietic populations (CD45+, CD34+, CD90) and functions for CFU-GEMM (GEMM) following culture with CXCL12, the CXCR4 inhibitor plerixafor, or nothing at all (NoRx) at 20% oxygen (n=3 donors per group). FIG. 4F graphically illustrates the fold change of the indicated immunophenotypic hematopoietic populations (CD45+, CD34+, CD90) and functions for CFU-GEMM (GEMM) following culture with CXCL12, the CXCR4 inhibitor plerixafor, or nothing at all (NoRx) at 1% oxygen (n=3 donors per group). FIG. 4G graphically illustrates the fold change of the indicated immunophenotypic hematopoietic populations (CD45+, CD34+, CD90) and functions of CFU-GEMM (GEMM) following culture with BMP4, Noggin, or no additive (NoRX) at 20% oxygen levels (n=3 donors per group). FIG. 4H graphically illustrates the fold change of the indicated immunophenotypic hematopoietic populations (CD45+, CD34+, CD90) and functions of CFU-GEMM (GEMM) following culture with BMP4, Noggin, or no additive (NoRX) at 1% oxygen levels (n=3 donors per group). Data are shown as mean±SD (* P<0.05, ** P<0.01. *** P<0.001, or if not shown, the comparison was not significant). FIG. 4I graphically illustrates the fold change of the indicated immunophenotypic hematopoietic populations (CD45+, CD34+, CD90) and functions CFU-GEMM (GEMM) is shown following culture with the indicated CXCL12, BMP4, Noggin, and/or Plerixafor additives at 20% oxygen (n=3 donors per group). FIG. 4J graphically illustrates the fold change of the indicated immunophenotypic hematopoietic populations (CD45+, CD34+, CD90) and functions CFU-GEMM (GEMM) is shown following culture with the indicated CXCL12, BMP4, Noggin, and/or Plerixafor additives at 1% oxygen (n=3 donors per group). Data are shown as mean±SD (* P<0.05, ** P<0.01, *** P<0.001, or if not shown, the comparison was not significant).

FIG. 5A-5H illustrate symmetric self-renewal of HSCs capable of long-term NSG engraftment. FIG. 5A schematically illustrates methods for single iHSC expansion and analysis. Each iHSC was propagated individually within a well and progeny in each well were analyzed independently either by flow cytometry or for their potential to engraft NSG mice. To test engraftment, a random subset of wells were split into two portions and transplanted into each of two NSG recipient mice. FIG. 5B graphically illustrates marrow engraftment of hCD45+ progeny from a cell expanded as described in FIG. 5A where the data are shown as percentage of femoral marrow cells. Progeny from expanded wells were split into two portions (A or B) and each portion was individually transplanted into an NSG mouse (n=2 mice per well, total n=16 mice). FIG. 5C graphically illustrates myeloid (CD14/CD15) and lymphoid (CD19/CD3) engraftment (% of hCD45) for each transplanted mouse. FIG. 5D graphically illustrates the total number of CD45+, CD34+, iHSC progeny cells from a single iHSC after 3 weeks of culture. FIG. 5E is a schematic illustrating a method for transduction of CD34+ aHSPCs and sorting of single transduced iHSCs per well in a 96-well format followed by 3 weeks of propagation. FIG. 5F graphically illustrates the percentage of iHSCs that remain GFP+ at the end of three weeks of culture of a single iHSC. FIG. 5G graphically illustrates the total number of GFP+CD45+ and GFP+CD34+ progeny from a single iHSC after 3 weeks of culture. FIG. 5H graphically illustrates the total number of GFP+iHSC progeny from a single iHSC after 3 weeks of culture. Data are shown as mean±SD (* P<0.05. ** P<0.01, *** P<0.001, or if not shown, the comparison was not significant).

FIG. 6 is a schematic diagram illustrating methods for transduction and transplantation of aHSPCs that allow assessment of the expansion of differently genetically-marked cells and determination of the genetically-marked cells' potential for long-term engraftment. Transduced cells were split into two portions. One portion was cryopreserved immediately while the other portion was cryopreserved after ex vivo expansion using the vascular niche. The cell types were later thawed. Expanded cells that had been transduced with GFP-lentiviral vector were mixed with unexpanded cells transduced with mCherry lentiviral vector. Expanded mCherry-expressing HSPCs were mixed with unexpanded GFP-expressing cells prior. The different cell mixtures were transplanted into separate mice. Engraftment was assessed in the blood and marrow of the mice 20 weeks after transplantation.

FIG. 7A-7C illustrate expansion and engraftment of genetically modified human sickle cell disease (SCD) peripheral blood HSPCs engineered to express non-sickling β-globin. FIG. 7A is a schematic diagram illustrating methods used to transduce aHSPCs mobilized from patients having sickle cell disease with non-sickling β-globin lentiviral expression vector. Transduced aHSPCs were either cryopreserved right away or cryopreserved after expansion using the vascular niche culture conditions. Thawed, transduced cells were transplanted into NSG mice. The peripheral blood (PB) and bone marrow of the mice were analyzed 20 weeks later. FIG. 7B graphically illustrates the percent of human CD45-expressing cells and the percent engraftment of the myeloid and lymphoid cells derived from the β-globin expressing, genetically modified aHSPCs in the peripheral blood of the mice. FIG. 7C graphically illustrates the percent of human CD45-expressing cells and the percent engraftment of the myeloid and lymphoid cells derived from the β-globin expressing, genetically modified aHSPCs in the marrow of the mice.

FIG. 8A-8B illustrate some exemplary methods for expanding HSPCs. FIG. 8A schematically illustrates expansion of HSPCs in culture with microcarriers that contain endothelial cells (e.g., E4-expressing endothelial cells, E4ECs, or endothelial cells that do not express E4). FIG. 8B graphically illustrates fold expansion when using a microcarrier-based system, either with static cultures or in spinner flasks. Cytodex-3® microcarriers (Cyt; dextran beads coated with denatured porcine-skin collagen bound to their surface) were used in this experiment to expand HSPCs under low oxygen, vascular niche culture conditions. The microcarrier-based systems can be upscaled 40-fold without modification.

FIG. 9 graphically illustrates that media containing only FGF2. EGF, IGF, HEP, or combinations thereof, can effectively promote the expansion of HSPCs even without E4ORF1 expression by endothelial feeder cells. However, use of FGF2 without EGF, IGF, HEP, or combinations thereof, was most effective. FGF2 can be used to replace the effect of E4 in a serum-free culture system that can be widely adapted.

FIG. 10A-IOC illustrate how mutant (e.g. cancer) stem cells can give rise to a plethora of different types of mutant (e.g., cancerous) differentiated hematopoietic cells and how to identify agents that can deplete or eliminate the mutant stem cells before they expand and differentiate into a full-blown myeloproliferative neoplasm (MPN). FIG. 10A is a schematic diagram illustrating the types of cells that can differentiate from hematopoietic stem cells (HSCs), and showing how a small number of myeloproliferative neoplasm stem and progenitor cells can expand to be a significant proportion of the differentiated hematopoietic cell population. FIG. 10B schematically illustrates a screening method for identifying useful treatment agents for depleting/eliminating myeloproliferative neoplasm (MPN) stem and progenitor cells (MPN-SPCs) from stem cells isolated from donor bone marrow or peripheral blood. For example the JAK2V617F mutation is an acquired, somatic mutation present in the majority of patients with myeloproliferative cancer (myeloproliferative neoplasms), and the chronic malignancy can begin with one mutated hematopoietic stem cell (HSC). Cells are harvested from a donor or patient. The harvest cells can include hematopoietic stem cells (HSCs), more differentiated hematopoietic cells, myeloproliferative neoplastic (MPN) stem cells (MPN-SCs), and myeloproliferative neoplastic (MPN) cells. The cells are plated with endothelial cells and cultured under the low oxygen conditions in the presence or absence of one or more test agents (study agents). The expansion of the different cell types is monitored, for example, by cell cytometry and/or polymerase chain reaction (PCR). Useful agents are identified as those that reduce or eliminate the myeloproliferative neoplasm (MPN) stem and progenitor cells (MPN-SPCs) from the other cells isolated from donor bone marrow or peripheral blood. FIG. 10C graphically illustrates that interferon-alpha (IFN) can reduce the expansion of MPN-SPCs in some but not all patient samples. Bone marrow or peripheral blood sampled were harvest from seven different polycythemia vera (PV) patients (each with a different MPD number). The hematopoietic stem cells (HSCs) from the different patients included mutant cells with the JAK2V617F mutation. Each graph shows the fold expansion, with interferon-alpha (IFN) or without it (None) in the culture media, for JAK2V617F mutant cells compared to the expansion of wild type (JAK2) cells from one of the seven polycythemia vera (PV) patients. Although interferon-alpha did reduce the proliferation of JAK2V617F mutant cells from some patients (e.g., patient MPD377), interferon-alpha did not deplete the mutant cells from other patients (e.g., patient MPD090) and the lack of previous interferon-alpha treatment did not always correlate with depletion of mutant cells (e.g., patient MPD216). Hence, the methods described herein are useful for identifying whether a particular treatment option may be useful, and which treatment agents and methods are most effective for individual patients.

FIG. 11 shows box plots illustrating the effects of different test agents on the expansion of the following cell types harvested from six different donors with myeloproliferative neoplasms (MPNs). CD45+ (referred to as “Heme”) cells, CD45+CD34+ non-EC cells (referred to as “CD34” cells); and CD31−CD45+CD34+CD38−CD45RA−CD90+ cells (referred to as HSC). As illustrated, test agent CPI-0610 (a BET-domain inhibitor) was most effective for reducing cell expansion of the Heme, CD34, and HSC cells, particularly when combined with interferon-alpha.

FIG. 12A-12C illustrate optimization of conditions for harvesting of cells from E4EC-loaded Corning Dissolvable Microcarriers. FIG. 12A graphically illustrates that optimal digestion and recovery of viable mono-cellular suspensions was obtained with pre-digestion of EC-loaded microcarriers by addition of Benzonase® (B) and Celase® (C) with stirring followed by addition of EDTA (E) and pectinase (P.) E4ECs loaded onto Corning dissolvable microcarriers were tested using various combinations of enzymes and procedures. As illustrated, pectinase and EDTA are required to digest the microcarrier polysaccharide matrix but other enzymes were required to optimize harvesting. Hence, Celase® (C) and/or TripLE® (T) were used to digest basement membranes, while Benzonase® (B) was used to help disaggregate cells by digesting free nucleic acids. FIG. 12B graphically illustrates the numbers of endothelial cells after growth on Corning Dissolvable Microcarriers (the ECs did adhere), followed by digestion as described in FIG. 12A, and seeding of the harvested cells in T175 flask. Cell morphology was appropriate and cell growth was essentially the same as ECs that had not been grown and then harvested from microcarriers. FIG. 12C graphically illustrates the fold change in cell numbers after use of EC-dissolvable microcarriers for expansion and after harvesting the cells. The fold change in CD45+ and CD34+ cells is shown, as well as the ability of the harvested cells to form colony forming units that generated myeloid cells (CFU-GEMM).

### DETAILED DESCRIPTION

Methods and compositions for expanding hematopoietic stem cells and hematopoietic progenitor cells are described herein. The methods are so efficient that they can be incorporated into screening methods using small patient HSC samples for identifying individualized treatment regimens and agents—regimens and agents that are uniquely effective for treatment of a particular patient with a particular type of disease or condition.

The methods described herein are fast. For example, within about one week 1 million CD34+ cells can be expanded to about 20-40 million CD34+ HSPCs. Single cells can be expanded by the methods described herein, and the methods are equally effective for wild type cells and for cells such as sickle cell anemia cells. The expanded cultures can also be maintained and their phenotypes can be preserved under the culture conditions described herein.

Human Hematopoietic Stem Cells and/or Hematopoietic Progenitor Cells

Hematopoietic stem cells and hematopoietic progenitor cells can be obtained from any donor. The hematopoietic stem cells and hematopoietic progenitor cells can be healthy, wild type cells or they can have mutations that can adversely affect their function. Any such cells can be expanded by the methods described herein. The methods described herein can be used to detect such mutations, and in some cases repair the mutations so that genetically repaired cells can then be expanded. In some cases the hematopoietic stem cells and hematopoietic progenitor cells are autologous to a person or patient who may later receive an expanded population of the hematopoietic stem cells and/or hematopoietic progenitor cells.

The hematopoietic stem cells and hematopoietic progenitor cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

Hematopoietic stem and progenitor cells (including adult hematopoietic stem cells and hematopoietic progenitor cells, aHSPCs) can be obtained from bone marrow or by mobilization of peripheral blood (PB). For example, bone marrow cells can be harvested from a donor under sterile conditions. In general, bone marrow is aspirated by needle from larger bones, such as the hip bone, femur, or chest bones.

Hematopoietic stem and progenitor cells can be mobilized and obtained from the peripheral blood. Mobilization is a process whereby the cells are stimulated out of the bone marrow space (e.g., from the hip bones and the chest bone) into the bloodstream so they are available for collection. Mozobil® (plerixafor) injection can be used for such mobilization. In some cases, Mozobil (plerixafor) can be used in combination with granulocyte-colony stimulating factor (G-CSF) to mobilize hematopoietic stem and progenitor cells to the peripheral blood. After collection the cells can be expanded immediately, or frozen and stored until expansion is desired.

### Endothelial Cell Niche

Hematopoietic stem cells and progenitor cells are notoriously difficult to maintain and grow in culture. However, expansion of such cells can be improved by use of endothelial “feeder cells.” The primary endothelial cells currently used are believed to require the presence of serum or growth factors for long-term maintenance. But many stem cells cannot tolerate the presence of serum or certain growth factors. Hence, serum and certain types of growth factors are not used for expansion of hematopoietic stem cells and hematopoietic progenitor cells in the methods described herein.

Instead, culture conditions are used that recreate the vascular niche that hematopoietic stem cells and hematopoietic progenitor cells naturally occupy in vivo. Such vascular niche includes living endothelial cells. In some cases, the endothelial cells can be mitotically inactive (e.g., by subjecting them to irradiation or mitomycin-C (MMC) exposure). However, in general, it is most useful to employ living, growing (mitotically active) endothelial cells.

Current methodology for expanding human bone marrow CD34+ cells requires transfection of endothelial cells with an E4ORF1. The E4 region of the adenoviral genome encodes several open reading frames (ORFs). However, the E4ORF1 gene has been found to provide certain biological effects on endothelial cells, such as promoting survival and inducing proliferation of endothelial cells and also stimulating angiogenesis.

As illustrated herein, in some cases E4ORF1-expressing endothelial cells can be useful feeder cells to support the growth of stem cells and progenitor cells such as hematopoietic stem or progenitor cells. A sequence for a human adenovirus type 5 E4 region (containing ORF1) is available in the NCBI database (see for example accession number D12587) and shown below as SEQ ID NO:1.

| | |
|---|---|
| 1 | ATGGCTGCCG CTGTGGAAGCGCTGTATGTT GTTCTGGAGC |
| 41 | GGGAGGGTGC TATTTTGCCTAGGCAGGAGG GTTTTTCAGG |
| 81 | TGTTTATGTG TTTTTCTCTCCTATTAATTT TGTTATACCT |
| 121 | CCTATGGGGG CTGTAATGTTGTCTCTACGC CTGCGGGTAT |
| 161 | GTCTTCCCCC GGGCTATTCGGTCGCTTTTT AGCACTGACC |
| 201 | GATGTGAATC AACCTGATGTGTTTACCGAG TCTTACATTA |
| 241 | TGCATCCGGA CATGACCGAGGAGCTGTCAG TGGTGCTTTT |
| 281 | TAATCACGGT GACCAGTTTTTTTACGGTCA CGCCGGCATG |
| 321 | GCCGTAGTCC GTCTTATGCTTATAAGGGTT GTTTTTCCTG |
| 361 | TTGTAAGACA GGCTTCTAATGTTTAA |

In some cases, the E4 region (containing ORF1) is an adenovirus 52 (Ad52) E4ORF1 sequence, shown below as SEQ ID NO:2.

| | |
|---|---|
| 1 | ATGGCTGATC AACATATATA TGTCCATTTG CTGGGACGTC |
| 41 | GGGCCTTTTT GCCCCAGCAA CAGGGTTATT CTAATATGTA |
| 81 | TGTTCTTTTT TCACCGGAGG ATTTTGTGCT TGCTCCCAGA |
| 121 | GGGATTATTT TGCTGTCTTT GCAGCTGTCG TTGGATATTC |
| 161 | CCACTGGGTA TCTGGGACGT TTTTTTTCTG TGGCGGATAT |
| 201 | GAATGTGAGG GGGGTATTGC TGTGTGCTCA GGAGATCCAA |
| 241 | CCGAGTACAT GGTGGGAAGT TTCTGTTGTT CTTTTTAATC |
| 281 | ATTCGGATGA ATTTTACCGC GGTTCCCGGG GACAGCCGGT |
| 321 | TGCTTGCCTG CTGCTGGAGC GTGTCATATA TCCCACCGTT |
| 361 | CGCCAAGCTT CTTTAGTTTA A |

The Ad52 SEQ ID NO:2 E4ORF1 nucleic acid encodes a polypeptide with the following sequence (SEQ ID NO:3).

| | |
|---|---|
| 1 | MADQHIYVHL LGRRAFLPQQ QGYSNMYVLE SPEDFVLAPR |
| 41 | GIILLSLQLS LDIPTGYLGR FFSVADMNVR GVLLCAQEIQ |
| 81 | PSTWWEVSVV LFNHSDEFYR GSRGQPVACL LLERVIYPTV |
| 121 | RQASLV |

In some cases, the E4 region (containing ORF1) is from an SFG_E4orf1_NYSTEM-RV sequence and has the following sequence (SEQ ID NO:4).

| | |
|---|---|
| 1 | ATGGCTGCCG CTGTGGAAGC GCTGTATGTT GTTCTGGAGC |
| 41 | GGGAGGGTGC TATTTTGCCT AGGCAGGAGG GTTTTTCAGG |
| 81 | TGTTTATGTG TTTTTCTCTC CTATTAATTT TGTTATACCT |
| 121 | CCTATGGGGG CTGTAATGTT GTCTCTACGC CTGCGGGTAT |
| 161 | GTATTCCCCC GGGCTATTTC GGTCGCTTTT TAGCACTGAC |
| 201 | CGATGTGAAT CAACCTGATG TGTTTACCGA GTCTTACATT |
| 241 | ATGACTCCGG ACATGACCGA GGAGCTGTCG GTGGTGCTTT |
| 281 | TTAATCACGG TGACCAGTTT TTTTACGGTC ACGCCGGCAT |
| 321 | GGCCGTAGTC CGTCTTATGC TTATAAGGGT TGTTTTTCCT |
| 361 | GTGT?!AGAC AGGCTTCTAA TGTTAA |

The SFG_E4orf1_NYSTEM-RV sequence (SEQ ID NO:4) nucleic acid encodes a polypeptide with the following sequence (SEQ ID NO:5′).

| | |
|---|---|
| 1 | MAAAVEALYV VLEREGAILP RQEGFSGVYV FFSPINFVIP |
| 41 | PMGAVMLSLR LRVCIPPGYF GRFLALTDVN QPDVFTESYI |
| 81 | MTPDMTEELS VVLFNHGDQF FYGHAGMAVV RLMLIRVVFP |
| 121 | VVRQASNV |

In some cases, the E4 region (containing ORF1) from the SFG_E4orf1_NYSTEM-RV sequence has a thymine (T) at position 26 instead of the adenine (A) shown in SEQ ID NO:4. This modified E4ORF1 sequence with the thymine (T) at position has the following sequence (SEQ ID NO:6).

| | |
|---|---|
| 1 | ATGGCTGCCG CTGTGGAAGC GCTGTTTGTT GTTCTGGAGC |
| 41 | GGGAGGGTGC TATTTTGCCT AGGCAGGAGG GTTTTTCAGG |
| 81 | TGTTTATGTG TTTTTCTCTC CTATTAATTT TGTTATACCT |
| 121 | CCTATGGGGG CTGTAATGTT GTCTCTACGC CTGCGGGTAT |
| 161 | GTATTCCCCC GGGCTATTTC GGTCGCTTTT TAGCACTGAC |
| 201 | CGATGTGAAT CAACCTGATG TGTTTACCGA GTCTTACATT |
| 241 | ATGACTCCGG ACATGACCGA GGAGCTGTCG GTGGTGCTTT |
| 281 | TTAATCACGG TGACCAGTTT TTTTACGGTC ACGCCGGCAT |
| 321 | GGCCGTAGTC CGTCTTATGC TTATAAGGGT TGTTTTTCCT |
| 361 | GTTGTAAGAC AGGCTTCTAA TGTTTAA |

The SFG_E4orf1_NYSTEM-RV sequence with the thymine (T) at position 26 (SEQ ID NO:6) encodes a polypeptide with the following sequence, where position 9 has a phenylalanine (SEQ ID NO:7) rather than the tyrosine present in SEQ ID NO:5.

```
    1 MAAAVEALFV VLEREGAILP RQEGFSGVYV FFSPINFVIP
  41 PMGAVMLSLR LRVCIPPGYF GRFLALTDVN QPDVFTESYI
  81 MTPDMTEELS VVLFNHGDQF FYGHAGMAVV RLMLIRVVFP
121 VVRQASNV
```

In some cases, the E4 region (containing ORF1) is from an E4ORF1_N-FLAG_pCCL sequence, with the following sequence (SEQ ID NO:8).

| | |
|---|---|
| 1 | ATGGACTACA AAGACGATCA CGACAAGGCT GCCGCTGTGG |
| 41 | AAGCGCTGTA TGTTGTTCTG GAGCGGGAGG GTGCTATTTT |
| 81 | GCCTAGGCAG GAGGGTTTTT CAGGTGTTTA TGTGTTTTTC |
| 121 | TCTCCTATTA ATTTTGTTAT ACCTCCTATG GGGGCTGTAA |
| 141 | TGTTGTCTCT ACGCCTGCGG GTATGTATTC CCCCGGGCTA |
| 161 | TTTCGGTCGC TTTTTAGCAC TGACCGATGT GAATCAACCT |
| 201 | GATGTGTTTA CCGAGTCTTA CATTATGACT CCGGACATGA |
| 241 | CCGAGGAGCT GTCGGTGGTG CTTTTTAATC ACGGTGACCA |
| 281 | GTTTTTTTAC GGTCACGCCG GCATGGCCGT AGTCCGTCTT |
| 321 | ATGCTTATAA GGGTTGTTTT TCCTGTTGTA AGACAGGCTT |
| 361 | CTAATGTTTA A |

The E4ORF1_N-FLAG_pCCL sequence (SEQ ID NO:8) nucleic acid encodes a polypeptide with the following sequence (SEQ ID NO:9).

| | |
|---|---|
| 1 | MDYKDDDDKA AAVEALYVVL EREGAILPRQ EGFSGVYVFF |
| 41 | SPINFVIPPM GAVMLSLRLR VCIPPGYFGR FLALTDVNQP |
| 81 | DVFTESYIMT PDMTEELSVV LFNHGDQFFY GHAGMAVVRL |
| 121 | MLIRVVFPVV RQASNV |

The E4ORF1 in the E4ORF1_N-FLAG_pCCL sequence has an N-terminal FLAG sequence with the following sequence: MDYKDDDDK (SEQ ID NO: 10).

The E4ORF1_N-FLAG_pCCL sequence can have a thymine (T) at position 50, rather than the adenine (A) highlighted in SEQ ID NO:8. This E4ORF1_N-FLAG_pCCL sequence with a thymine (T) at position 50 is shown below as SEQ ID NO: 11.

| | |
|---|---|
| 1 | ATGGACTACA AAGACGATGA CGACAAGGCT GCCGCTGTGG |
| 41 | AAGCGCTGTT TGTTGTTCTG GAGCGGGAGG GTGCTATTTT |
| 81 | GCCTAGGCAG GAGGGTTTTT CAGGTGTTTA TGTGTTTTTC |
| 121 | TCTCCTATTA ATTTTGTTAT ACCTCCTATG GGGGCTGTAA |
| 141 | TGTTGTCTCT ACGCCTGCGG GTATGTATTC CCCCGGGCTA |
| 181 | TTTCGGTCGC TTTTTAGCAC TGACCGATGT GAATCAACCT |
| 201 | GATGTGTTTA CCGAGTCTTA CATTATGACT CCGGACATGA |
| 241 | CCCAGCACCT GTCGGTGGTG CTTTTTAATC ACGGTGACCA |
| 281 | GTTTTTTTAC GGTCACGCCG GCATGGCCGT AGTCCGTCTT |
| 321 | ATGCTTATAA GGGTTGTTTT TCCTGTTGTA AGACAGGCTT |
| 361 | CTAATGTTTA A |

The E4ORF1_N-FLAG_pCCL sequence with the thymine (1′) at position 50 (SEQ ID NO:11 encodes a polypeptide with the following sequence, where position 17 has a phenylalanine (SEQ ID NO: 12) rather than the tyrosine present in SEQ ID NO:9.

```
    1 MDYKDDDDKA AAVEALFVVL EREGAILPRQ EGFSGVYVFF
  41 SPINFVIPPM GAVMLSLRLR VC1PPGYFGR FLALTDVNQP
  81 DVFTESYIMT PDMTEELSVV LFNHGDQFFY GHAGMAVVRL
121 MLIRVVFPVV RQASNV
```

In some cases, the E4 region (containing ORF1) is from a pCCL-GAGm3-PGK-Ad52_WPREm6 vector, where the E4ORF1 sequence is as follows (SEQ ID NO:13).

| | |
|---|---|
| 1 | ATGGCTGATC AACATATATA TGTGCATTTG CTGGGACGTC |
| 41 | GGGCCTTTTT GCCCCAGCAA CAGGGTTATT CTAATATGTA |
| 81 | TGTTCTTTTT TCACCGGAGG ATTTTGTGCT TGCTCCCAGA |
| 121 | GGGATTATTT TGCTGTCTTT GCAGCTGTCG TTGGATATTC |
| 161 | CCACTGGGTA TCTGGGACGT TTTTTTTCTG TGGCGGATAT |
| 201 | GAATGTGAGG GGGGTATTGC TGTGTGCTCA GGAGATCCAA |
| 241 | CCGAGTACAT GGTGGGAAGT TTCTGTTGTT CTTTTTAATC |
| 281 | ATTCGGATGA ATTTTACCGC GGTTCCCGGG GACAGCCGGT |
| 321 | TGCTTGCCTG CTGCTGGAGC GTGTCATATA TCCCACCGTT |
| 361 | CGCCAAGCTT CTTTAGTTTA A |

The E4ORF1 sequence (SEQ ID NO:13) from pCCL-GAGm3-PGK-Ad52_WPREm6 encodes a polypeptide with the following sequence (SEQ ID NO:14).

| | |
|---|---|
| 1 | MADQHIYVHL LGRRAFLPQQ QGYSNMYVLF SPEDFVLAPR |
| 41 | GIILLSLQLS LDIPTGYLGR FFSVADMNVR GVLLCAQEIQ |
| 81 | PSTWWEVSVV LFNHSDEFYR GSRGQPVACL LLERVIYPTV |
| 121 | RQASLV |

In certain embodiments, the E4ORF1 gene used is the whole adenovirus E4ORF1 gene, or a variant, mutant or fragment thereof that has improved functional properties described herein. For example, the variant or mutant of the E4ORF1 sequence can be a sequence with about an 85% identity to any of SEQ ID NOs:1-14, or about an 88%, 89%, 90%, 91%, 92%, 93%, 94%, 95%, 96%, 97%, 98%, or 99% sequence identity to SEQ ID NO:1 that retains the ability to enhance expansion of hematopoietic stem cells and/or hematopoietic progenitor cells. In some cases, a fragment of an E4ORF1 is used. For example, the E4ORF1 sequence can vary in length by ±30 nucleotides relative to any of SEQ ID NOs:1-9 or 11-14; or about ±28 nucleotides, ±26 nucleotides, ±24 nucleotides, ±22 nucleotides, ±20 nucleotides, ±18 nucleotides, ±16 nucleotides, ±14 nucleotides, ±12 nucleotides, ±10 nucleotides, ±9 nucleotides, ±8 nucleotides, ±7 nucleotides, ±6 nucleotides, ±5 nucleotides, ±4 nucleotides, ±3 nucleotides, ±2 nucleotides, or ±1 nucleotides relative to any of SEQ ID NO:1-9 or 11-14, all of which retain the properties described herein, including, but not limited to, the ability to enhance expansion of hematopoietic stem cells and/or hematopoietic progenitor cells and the ability to promote survival of hematopoietic stem cells and/or hematopoietic progenitor cells.

One goal of the inventors was to find a way to expand human hematopoietic stem cells and/or hematopoietic progenitor cells without the need for E4ORF1 transfection of endothelial cells. Hence, in some cases endothelial cells are used that are not modified to include an adenoviral E4 sequence such as the E4ORF1. Such endothelial cells do not have any E4 nucleic acids (and no E4ORF1 nucleic acids) and do not express any E4 polypeptides (and no E4ORF1 polypeptides).

In order to achieve that goal, the inventors discovered new, optimized culture conditions to grow endothelial cells in serum-free media for at least seven days while avoiding detachment or death of the endothelial cells. The inventors evaluated different cytokines and other additives with endothelial cells in culture media used for expansion of hematopoietic stem cells and/or hematopoietic progenitor cells. Examples of cytokines and additives that can be used with endothelial cells in hematopoietic stem/progenitor cell expansion media include FGF, FGF2, EGF, IGF (e.g., all at about 10 ng/mL). Examples of additives that can be used with endothelial cells in hematopoietic stem/progenitor cell expansion media include KITL, THPO, FLT3L, heparin, SR1 (BioVision Technologies), UM171 (BioVision Technologies), N-Acetylcysteine (NAC) (Pfizer), BAY 87-2243 (Selleck Chem), IOX2 (Selleck Chem), CXCL12 (PeproTech), BMP4 (PeproTech), Plerixafor (PeproTech), Noggin (Thermo), or combinations thereof.

The inventors found that growing cells in hypoxia (low oxygen) with fibroblast growth factor (FGF2) gave excellent results—these conditions helped the endothelial cells to survive and support expansion of human hematopoietic stem cells and/or hematopoietic progenitor cells without the need for addition of other cytokines or additives. For example, the inventors found that after 7 days of culture, the fold expansion of human hematopoietic stem cells and/or hematopoietic progenitor cells in the absence of E4ORF1 while using low oxygen levels and FGF2 alone was similar to expansion of such cells in the presence of endothelial cells that express E4ORF1, FGF2 can therefore be used to replace the effect of E4 in a serum-free culture system that can be widely adapted.

However, in some cases, the growth and functioning of the hematopoietic stem cells and/or hematopoietic progenitor cells is optimized by using optimal ratios of endothelial cells to hematopoietic stem cells and/or hematopoietic progenitor cell (EC:HSPC ratios). Hence, varying ratios of endothelial cells to hematopoietic stem cells and/or hematopoietic progenitor cells can be used. For example, when seeding a culture to begin expansion, use of HSPC:EC ratios of 1:3 or more can lead to deterioration of expansion performance. In other words, use of greater numbers of endothelial cells relative to the numbers of hematopoietic stem cells and/or hematopoietic progenitor cells can improve expansion. For example, if a HSPC:EC ratio of 1:2 is used, the expansion may not be as robust as when a HSPC:EC ratio of 1:5 is used. HSPC:EC ratios of 1:5 or below provide good expansion results and there is no penalty for very low HSPC:EC ratios such as 1:15,000. Hence, the HSPC:EC ratio can be less than 1:3, or less than 1:4, or less than 1:5, or less than 1:10, or less than 1:20, or less than 1:100, or less than 1:200, or less than 1:1000, or less than 1:2000, or less than 1:3000, or less than 1:5000, or less than 1:10,000.

The endothelial cells can be cultured with hematopoietic stem cells and/or hematopoietic progenitor cells under conditions that allow ready separation of the hematopoietic stem cells and hematopoietic progenitor cells from the endothelial cells.

For example, the endothelial cells can be separate from the hematopoietic stem cells and hematopoietic progenitor cells, the endothelial cells can adhere to a solid surface (while the hematopoietic stem cells and hematopoietic progenitor cells remain suspended in the culture medium), or the endothelial cells can be readily distinguishable from the hematopoietic stem cells and/or hematopoietic progenitor cells (e.g., by cell sorting).

In some cases, microcarriers can be used to house the endothelial while the hematopoietic stem cell and/or hematopoietic progenitor cell populations are expanded in culture. Microcarriers are typically 125-250 micrometer spheres that have holes and crevices where cells can grow. The density of microcarriers allows them to be maintained in suspension with gentle stirring. Microcarriers can be made from a number of different materials including DEAE-dextran, glass, polystyrene plastic, acrylamide, collagen, pectin, and alginate. Preliminary testing indicates that microcarriers containing endothelial cells can provide the highest fold expansion of hematopoietic stem cells and/or hematopoietic progenitor cells. In some cases, the microcarrier used can be entirely dissolvable using enzymes such as collagenase, neutral proteinase, trypsin and pectinase. Dissolvable microcarriers can be entirely removed during harvest of expanded HSPCs using the vascular niche. The inventors have developed processes for removal of microcarriers after expansion. Such microcarrier-based expansion is efficient, inexpensive and useful for expansion of therapeutically effective numbers of cells, even if the cells are fragile and genetic modification is needed to repair a genetic defect in the cells.

Sealed systems can be used for expansion of the hematopoietic stem cells and/or hematopoietic progenitor cells. For example, while the cells can be expanded in plates over a layer of endothelial cells, but the cells can also be expanded in hollow fibers, or in reactors where the conditions can be controlled and monitored.

### Hypoxic Conditions

The hematopoietic stem cell and/or hematopoietic progenitor cell populations can be expanded under hypoxic conditions. Atmospheric oxygen and reactive oxygen species (ROS) can reduce the expansion of hematopoietic stem cells and/or hematopoietic progenitor cells significantly. When oxygen and reactive oxygen species are maintained at low levels, such as when hypoxic conditions are maintained, HIF1α expression is induces, which stimulates HIF1α-related signaling pathways in the cells, thereby promoting HSC self-renewal.

Hypoxic conditions can be achieved by using low oxygen atmospheric conditions and/or by using reactive oxygen scavengers.

For example, hypoxic conditions can involve use of an atmosphere and/or the culture media with less than 21% oxygen. In some cases, the atmosphere or the culture media can have 5% or less oxygen, 3% or less oxygen, or 2.5% or less oxygen, or 2% or less oxygen, or 1% or less oxygen.

As used herein, the phrase “normoxic conditions” refer to culture conditions of more than 5% oxygen or more than 3% oxygen. In some embodiments, “normoxic conditions” refer to about 5%, about 8%, about 10%, about 15%, or ambient/atmospheric conditions of about 20% oxygen.

Oxygen readily dissolves in water. The inventors directly measured dissolved oxygen in media and found that dissolved oxygen rapidly equilibrated with the surrounding atmospheric gases. Hence, when using a culture apparatus that is open to a hypoxic atmosphere, degassing the media may not be needed prior to seeding the cells because the media has already quickly equilibrated with a hypoxic atmosphere. However, the culture apparatus should be prepared and used for cell expansion within a closed, regulated system, such as a glove box, where the atmospheric gas content can be controlled and maintained as hypoxic.

One example of a glove box that can be used is a Biospherix cGMP glove box (see, e.g., biospherix.com/cell-therapy/). Other types of glove boxes can also be used.

In addition, reducing agents can be included in the media, buffers and other liquids that may be used to manipulate or expand the HSPCs. This prevents formation reactive oxygen species (ROS) and damage to the cells that can be caused by ROS. Cells are also kept cool when not being cultured for expansion. For example, the cells can be kept in a refrigerator (between about 0° C. and 40° C.) or on ice prior to and after expansion, and during analysis.

Examples of reducing agents that can be used include N-acetylcysteine, beta-mercaptoethanol, dithiothreitol, Tris (2-carboxyethyl) phosphine hydrochloride, (TCEP), and the like. As illustrated herein N-acetylcysteine (NAC), for example, can help reduce reactive oxygen species and improve cellular expansion.

Reactive oxygen species can also be reduced in the culture media to improve expansion of hematopoietic stem cells and/or hematopoietic progenitor cells. In some cases, cellular expansion can be achieved under normoxic atmospheric conditions so long as reactive oxygen species are reduced or eliminated in the culture media. Reduction in reactive oxygen scavengers can be achieved by use of one or more ROS scavengers. Examples of ROS scavengers that can be employed include N-acetyl-L-cysteine (NAC), Sodium pyruvate, N,N′-dimethylthiourea (DMTU), Trolox (6-hydroxy-2,5,7,8-tetramethylchroman-2-carboxylic acid), α-tocopherol, Ebselen (2-phenyl-1,2-benzisoselenazol-3(2H)-one), uric acid, Vitamin C (ascorbic acid), L-galactonic acid-g-galactose, imidazole, MnTBAP (manganese(III)-tetrakis(4-benzoic acid)porphyrin), and combinations thereof.

The concentration of ROS scavengers employed in culture media can vary. For example, ROS scavengers can be used at concentrations of at least 0.01 micromolar, at least 0.1 micromolar, at least 1.0 micromolar, at least 10 micromolar, at least 100 micromolar, at least 150 micromolar, at least 200 micromolar, or at least 300 micromolar. When ROS scavenging enzymes are use (e.g., catalase), the enzymes can be used at concentrations of at least 0.01 Units/ml, at least 0.1 Units/ml, at least 0.5 Units/ml, at least 1.0 Units/ml, at least 2.0 Units/ml, at least 3.0 Units/ml, at least 5.0 Units/ml, at least 7.0 Units/ml, at least 10 Units/ml, at least 15 Units/ml, at least 20 Units/ml, at least 30 Units/ml, at least 40 Units/ml, at least 50 Units/ml, at least 75 Units/ml or at least 100 U/ml.

When ROS scavengers are employed in culture media the atmospheric can be maintained during cellular expansion at oxygen levels higher than hypoxic atmospheric levels. For example, the atmospheric oxygen levels can be maintained at 3% or higher, or at 4% or higher, or at 5% or higher, or at 6% or higher, or at 7% or higher, or at 8% or higher, or at 9% or higher, or at 10% or higher, or at 15% or higher.

Genetic Modification of Hematopoietic Stem and/or Progenitor Cells

The hematopoietic stem cells and/or hematopoietic progenitor cells can be collected from donors suffering from a disease or condition, and then modified to alleviate the symptoms or progression of the disease or condition. For example, when a genetic defect is correlated with a disease and condition, hematopoietic stem cell and/or hematopoietic progenitor cell can be collected from a subject (donor) with the disease or condition, the genetic defect can be repaired, and the genetically repaired hematopoietic stem cells and/or hematopoietic progenitor cells can be expanded. A population of the expanded, repaired cells can then be administered to the subject (donor).

Cells can be genetically modified by any convenient method. Non-limiting examples of methods of introducing a modification into the genome of a cell can include use of microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety. For example, nucleases such as Cas nucleases, zinc finger nucleases (ZFNs), transcription activator like effector nucleases (TALENs), and/or meganucleases can be employed with a guide nucleic acid that allows the nuclease to target the genomic locus associated with a genetic disease or a condition.

A targeting vector can be used to repair a genetic defect. A “targeting vector” is a vector generally has a 5′ flanking region and a 3′ flanking region homologous to segments of the gene of interest. The 5′ flanking region and a 3′ flanking region can surround a DNA sequence comprising a modification and/or a repair DNA sequence to be inserted/substituted within a selected genetic defect. In some cases, the targeting vector does not comprise a selectable marker. However, such a selectable marker can facilitate identification and selection of cells with desirable genetic modifications. Examples of suitable selectable markers include antibiotics resistance genes such as chloramphenicol resistance, gentamycin resistance, kanamycin resistance, spectinomycin resistance (SpecR), neomycin resistance gene (NEO), and/or the hygromycin β-phosphotransferase genes. The 5′ flanking region and the 3′ flanking region can be homologous to regions within the gene, or to regions flanking the gene to be deleted, modified, or replaced with the unrelated DNA sequence.

The targeting vector is contacted with the native gene of interest in vivo (e.g., within the cell) under conditions that favor homologous recombination. For example, the cell can be contacted with the targeting vector under conditions that result in transformation of the cell(s) with the targeting vector.

A typical targeting vector contains nucleic acid fragments of not less than about 0.1 kb nor more than about 10.0 kb from both the 5′ and the 3′ ends of the genomic locus which encodes the gene or locus to be modified. These two fragments are separated by an intervening fragment of nucleic acid which encodes the modification to be introduced. When the resulting construct recombines homologously with the chromosome at this locus, it results in the introduction of the modification.

In some cases, a Cas9/CRISPR system can be used to repair a genetic defect. Clustered regularly interspaced short palindromic repeats (CRISPR)/CRISPR-associated (Cas) systems are useful for, e.g. RNA-programmable genome editing (see e.g., Marraffini & Sontheimer, Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is described, for example, by Mali et al. Science 2013 339:823-6; which is incorporated by reference herein in its entirety. Kits for the design and use of CRISPR-mediated genome editing are commercially available. e.g. the PRECISION X CAS9 SMART NUCLEASE™ System (Cat No. CAS900A-1) from System Biosciences, Mountain View, Calif.

In other cases, a cre-lox recombination system of bacteriophage P1, described by Abremski et al. 1983. Cell 32:1301 (1983), Sternberg et al., Cold Spring Harbor Symposia on Quantitative Biology, Vol. XLV 297 (1981) and others, can be used to promote recombination and alteration of a genetic defect. The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317 and 5,801,030), and in viral vectors (Hardy et al., J. Virology 71:1842 (1997).

The genomic modifications so incorporated can alter one or more nucleotides or encoded amino acid codons in a selected genetic locus. Such genomic modifications can improve the functioning of gene products by at least 10%, or at least 15%, or at least 20%, or at least 25%, or at least 30%, or at least 35%, or at least 40%, or at least 45%, or at least 50%, or at least 60%, or at least 70%, or at least 75%, or at least 80%, or at least 85%, or at least 90%, or at least 95%, or at least 97%, or at least 99%, compared to the unmodified (genetically defective) gene product expression or functioning.

For example, the methods described herein can be used to facilitate identification, and in some cases repair, of genetic defects associated with diseases and conditions. Examples of diseases and conditions that may be alleviated by genetic modification include hematological diseases, metabolism, HIV infection, toxicity associated with chemotherapy, and others.

Examples of hematological diseases that may be identified and/or treated include sickle cell anemias, the unstable hemoglobinopathies, hemoglobinopathies associated with polycythemia or with methemoglobinemia, α-thalassemia, and β-thalassemia.

“Hemoglobinopathy” is an umbrella term used to describe a group of genetic diseases that affect the body's hemoglobin. Hemoglobin is the protein in red blood cells that carries oxygen. It is made up of 2 alpha globin chains and 2 beta globin chains. Sickle cell disease is a hemoglobinopathy caused by a mutation in the beta globin gene, resulting in an abnormal hemoglobin called sickle hemoglobin, or Hb S. Different types of hemoglobinopathies arise, for example, when the hemoglobin beta S gene is inherited with another beta S gene, or when a different beta gene mutation is present, or when mutations of an alpha globin gene are present.

The alpha (HBA) and beta (HBB) loci determine the structure of the 2 types of polypeptide chains in adult hemoglobin, Hb A. The normal adult hemoglobin tetramer consists of two alpha chains and two beta chains. Mutant beta globin causes sickle cell anemia. Absence of beta chain causes beta-zero-thalassemia. Reduced amounts of detectable beta globin causes beta-plus-thalassemia. The order of the genes in the beta-globin cluster is 5′-epsilon-gamma-G-gamma-A-delta-beta-3′. Examples of hemoglobin beta globin amino acid sequences are available, for example, from the NCBI and Uniprot databases.

One example of a human hemoglobin beta globin amino acid sequence has NCBI accession no. NP_000509.1 (Uniprot P68871), which is shown below as SEQ ID NO:15.

```
    1 MVHLTPEEKS AVTALWGKVN VDEVGGEALG RLLVVYPWTQ
  41 RFFESFGDLS TPDAVMGNPK VKAHGKKVLG AFSDGLAHLD
  81 NLKGTFATLS ELHCDKLHVD PENFRLLGNV LVCVLAHHFG
121 KEFTPPVQAA YQKVVAGVAN ALAHKYH
```

A nucleotide (cDNA) sequence for the SEQ ID NO:15 beta globin polypeptides is shown below as SEQ ID NO: 16 (NCBI accession no. NM_000518.5).

```
    1 ATGGTGCATC TGACTCCTGA GGAGAAGTCT GCCGTTACTG
  41 CCCTGTGGGG CAAGGTGAAC GTGGATGAAG TTGGTGGTGA
  81 GGCCCTGGGC AGGCTGCTGG TGGTCTACCC TTGGACCCAG
121 AGGTTCTTTG AGTCCTTTGG GGATCTGTCC ACTCCTGATG
161 CTGTTATGGG CAACCCTAAG GTGAAGGCTC ATGGCAAGAA
201 AGTGCTCGGT GCCTTTAGTG ATGGCCTGGC TCACCTGGAC
241 AACCTCAAGG GCACCTTTGC CACACTGAGT GAGCTGCACT
281 GTGACAAGCT GCACGTGGAT CCTGAGAACT TCAGGCTCCT
321 GGGCAACGTG CTGGTCTGTG TGCTGGCCCA TCACTTTGGC
361 AAAGAATTCA CCCCACCAGT GCAGGCTGCC TATCAGAAAG
401 TGGTCGCTGG TGTGGCTAAT GCCCTGGCCC ACAAGTATCA
441 CTA
```

For example, mutations within the beta globin gene, or loss of the beta globin gene, in cells of a subject can readily be identified by observing the phenotype of the subject and/or by sequencing of the subject's genome. After identification, hematopoietic stem and progenitor cells having genetic defect(s) can be collected from a subject and the defect(s) in the beta globin gene can be repaired, for example by using the genetic modification methods described herein. One or more of the repaired cells can then be expanded to generate a population of repaired cells that can be administered for engraftment in the subject who originally donated the hematopoietic stem and progenitor cells.

In some cases, various metabolic diseases and conditions can be identified and genetic defects associated with the metabolic diseases and conditions can be repaired or altered in a subject's hematopoietic stem and progenitor cells. After expansion of the hematopoietic stem and progenitor cells, they can be introduced back into the subject to treat the disease or condition.

Examples of metabolic diseases and conditions that are correlated with a genetic defect include, familial hypercholesterolemia, Gaucher disease, Hunter syndrome. Krabbe disease, Maple syrup urine disease, Metachromatic leukodystrophy, mitochondrial encephalopathy lactic acidosis stroke-like episodes (MELAS), Niemann-Pick, phenylketonuria (PKU), porphyria, Tay-Sachs disease, Wilson's disease

In some cases, the HIV receptor CCR5 can be knocked out in hematopoietic stem and progenitor cells from a subject. Such CCR5-knockout cells can be administered to a subject to treat HIV infection or to make a subject resistant to HIV infection.

One example of a human CCR5 amino acid sequence that can be knocked out (e.g., deleted or mutated so it is not functional) is the human CCR5 polypeptide sequence (NCBI accession no. NP_001381712.1), shown below as SEQ ID NO: 17,

| | |
|---|---|
| 1 | MDYQVSSPIY DINYYTSEPC QKINVKQIAA RLLPPLYSLV |
| 41 | FIFGFVGNML VILILINCKR LKSMTDIYLL NLAISDLFFL |
| 81 | LTVPFWAHYA AAQWDFGNTM CQLLTGLYFI GFFSGIFFII |
| 121 | LLTIDRYLAV VHAVFALKAR TVTEGWTSV ITWVVAVFAS |
| 161 | LPGIIFTRSQ KEGLHYTCSS HFPYSQYQFW KNFQTLKIVI |
| 201 | LGLVLPLLVM VICYSGILKT LLRCRNEKKR HRAVRLIFTI |
| 241 | MIVYFLFWAP YNIVLLLNTE QEFFGLNNCS SSNRLDQAMQ |
| 281 | VTETLGMTHC CINPIIYAFV GEKFRNYLLV FFQKHIAKRF |
| 321 | CKCCSIFOQE APERASSVYT RSTGEQEISV GL |

The human CCR5 gene on chromosome is at position NC_000003.12 (46370142 . . . 46376206). This location can be partly or completely deleted. In some cases, one or more point mutations or a series of mutations can be introduced into such a CCR5 gene. A nucleotide (cDNA) sequence encoding the human CCR5 polypeptide with the SEQ ID NO: 17 sequence, for example, has NCBI accession no. NM_001394783.1 and is shown below as SEQ ID NO: 18.

| 1 | AGAAGAGCTG AGACATCCGT TCCCCTACAA GAAACTCTCC |
|---|---|
| 41 | CCGGGTGGAA CAAGATGGAT TATCAAGTGT CAAGTCCAAT |
| 81 | CTATGAGATC AATTATTATA CATCGGAGCC CTGCCAAAAA |
| 121 | ATCAATGTGA AGCAAATCGC ACCCCGCCTC CTGCCTCCGC |
| 161 | TCTACTCACT GGTGTTCATC TTTGGTTTTG TGGGCAACAT |
| 201 | GCTGGTCATC CTCATCCTGA TAAACTGCAA AAGGCTGAAG |
| 241 | AGCATGACTG ACATCTACCT GCTCAACCTG GCCATCTCTG |
| 281 | ACCTGTTTTT CCTTCTTACT GTCCCCTTCT GGGCTCACTA |
| 321 | TGCTGCCGCC CAGTGGGACT TTGGAAATAC AATGTGTCAA |
| 361 | CTCTTGACAG GGCTCTATTT TATAGGCTTG TTCTCTGGAA |
| 401 | TCTTCTTCAT CATCCTCCTG ACAATCGATA GGTACCTGGC |
| 441 | TGTCGTCCAT GCTGTGTTTG CTTTAAAAGC CAGGACGGTC |
| 481 | ACCTTTGGGG TGGTGACAAG TGTGATCACT TGGGTGGTGG |
| 521 | CTGTGTTTGC GTCTCTCCCA GGAATCATCT TTACCAGATC |
| 561 | TCAAAAAGAA GGTCTTCATT ACACCTGCAG CTCTCATTTT |
| 601 | CCATACAGTC AGTATCAATT CTGGAAGAAT TTCCAGACAT |
| 641 | TAAAGATAGT CATCTTGGGG CTGGTCCTGC CGCTGCTTGT |
| 681 | CATGGTCATC TGCTACTCGG GAATCCTAAA AACTCTGCTT |
| 721 | CGGTGTCGAA ATGAGAAGAA GAGGCACAGG GCTGTGAGGC |
| 761 | TTATCTTCAC CATCATGATT GTTTATTTTC TCTTCTGGGC |
| 801 | TCCCTACAAC ATTGTCCTTC TCCTGAACAC CTTCCAGGAA |
| 841 | ttctttggcc TGAATAATTG CAGTAGCTCT AACAGGTTGG |
| 881 | ACCAAGCTAT GCAGGTGACA GAGACTCTTG GGATGACGCA |
| 921 | CTGCTGCATC AACCCCATCA TCTATGCCTT TGTCGGGGAG |
| 961 | AAGTTCAGAA ACTACCTCTT AGTCTTCTTC CAAAAGCACA |
| 1001 | TTGCCAAACG CTTCTGCAAA TGCTGTTCTA TTTTCCAGCA |
| 1041 | AGAGGCTCCC GAGCGAGCAA GCTCAGTTTA CACCCGATCC |
| 1081 | ACTGGGGAGC AGGAAATATC TGTGGGCTTG TGACACGGAC |
| 1121 | TCAAGTGGGC TGGTGACCCA GTCAGAGTTG TGCACATGGC |
| 1161 | TTAGTTTTCA TACACAGCCT GGGCTGGGGG TGGGGTGGGA |
| 1201 | GAGGTCTTTT TTAAAAGGAA GTTACTGTTA TAGAGGGTCT |
| 1241 | AAGATTCATC CATTTATTTG GCATCTGTTT AAAGTAGATT |
| 1281 | AGATCTTTTA AGCCCATCAA TTATAGAAAG CCAAATCAAA |
| 1321 | ATATGTTGAT GAAAAATAGC AACCTTTTTA TCTCCCCTTC |
| 1361 | ACATGCATCA AGTTATTGAC AAACTCTCCC TTCACTCCGA |
| 1401 | AAGTTCCTTA TGTATATTTA AAAGAAAGCC TCAGAGAATT |
| 1441 | GCTCATTCTT GAGTTTAGTG ATCTGAACAG AAATACCAAA |
| 1481 | ATTATTTCAG AAATGTACAA CTTTTTACCT AGTACAAGGC |
| 1521 | AACATATAGG TTGTAAATGT GTTTAAAACA GGTCTTTGTC |
| 1561 | TTGCTATGGG GAGAAAAGAd ATGAATATGA TTAGTAAAGA |
| 1601 | AATGACACTT TTCATGTGTG ATTTCCCCTC CAAGGTATGG |
| 1641 | TTAATAAGTT TCACTGACTT AGAACCAGGC GAGAGACTTG |
| 1681 | TGGCCTGGGA GAGCTGGGGA AGCTTCTTAA ATGAGAAGGA |
| 1721 | ATTTGAGTTG GATCATCTAT TGCTGGCAAA GACAGAAGCC |
| 1761 | TCACTGCAAG CACTGCATGG GCAAGCTTGG CTGTAGAAGG |
| 1801 | AGACAGAGCT GGTTGGGAAG ACATGGGGAG GAAGGACAAG |
| 1841 | GCTAGATCAT GAAGAACCTT GACGGCATTG CTCCGTCTAA |
| 1881 | GTCATGAGCT GAGCAGGGAG ATCCTGGTTG GTGTTGCAGA |
| 1921 | AGGTTTACTC TGTGGCCAAA GGAGGGTCAG GAAGGATGAG |
| 1961 | CATTTAGGGC AAGGAGACCA CCAACAGCCC TCAGGTCAGG |
| 2001 | GTGAGGATGG CCTCTGCTAA GCTCAAGGCG TGAGGATGGG |
| 2041 | AAGGAGGGAG GTATTCGTAA GGATGGGAAG GAGGGAGGTA |
| 2081 | TTCGTGCAGC ATATGAGGAT GCAGAGTCAG GAGAACTGGG |
| 2121 | GTGGATTTGG GTTGGAAGTG AGGGTCAGAG AGGAGTCAGA |
| 2161 | GAGAATCCCT AGTCTTCAAG CAGATTGGAG AAACCCTTGA |
| 2201 | AAAGACATCA AGCACAGAAG GAGGAGGAGG AGGTTTAGGT |
| 2241 | CAAGAAGAAG ATGGATTGGT GTAAAAGGAT GGGTCTGGTT |
| 2281 | TGCAGAGCTT GAACACAGTC TCACCCAGAC TCCAGGCTGT |
| 2321 | CTTTCACTGA ATGCTTCTGA CTTCATAGAT TTCCTTCCCA |
| 2361 | TCCCAGCTGA AATACTGAGG GGTCTCCAGG AGGAGACTAG |
| 2401 | ATTTATGAAT ACACGAGGTA TGAGGTCTAG GAACATACTT |
| 2441 | CAGCTCACAC ATGAGATCTA GGTGAGGATT GATTACCTAG |
| 2481 | TAGTCATTTC ATGGGTTGTT GGGAGGATTC TATGAGGCAA |
| 2521 | CCACAGGCAG CATTTAGCAC ATACTACACA TTCAATAAGC |
| 2561 | ATCAAACTCT TAGTTACTCA TTCAGGGATA GCACTGAGCA |
| 2601 | AAGCATTGAG CAAAGGGGTC CCATAGAGGT GAGGGAAGCC |
| 2641 | TGAAAAACTA AGATGCTGCC TGCCCAGTGC ACACAAGTGT |
| 2681 | AGGTATCATT TTCTGCATTT AACCGTCAAT AGGCAAAGGG |
| 2721 | GGGAAGGGAC ATATTCATTT GGAAATAAGC TGCCTTGAGC |
| 2761 | CTTAAAACCC ACAAAAGTAC AATTTACCAG CCTCCGTATT |
| 2801 | TCAGACTGAA TGGGGGTGGG GGGGGCGCCT TAGGTACTTA |
| 2841 | TTCCAGATGC CTTCTCCAGA CAAACCAGAA GCAACAGAAA |
| 2881 | AAATCGTCTC TCCCTCCCTT TGAAATGAAT ATACCCCTTA |
| 2921 | GTGTTTGGGT ATATTCATTT CAAAGGGAGA GAGAGAGGTT |
| 2961 | TTTTTCTGTT CTGTCTCATA TGATTGTGCA CATACTTGAG |
| 3001 | ACTGTTTTGA ATTTGGGGGA TGGCTAAAAC CATCATAGTA |
| 3041 | CAGGTAAGGT GAGGGAATAG TAAGTGGTGA GAACTACTCA |
| 3081 | GGGAATGAAG GTGTCAGAAT AATAAGAGGT GCTACTGACT |
| 3121 | TTCTCAGCCT CTGAATATGA ACGGTGAGCA TTGTGGCTGT |
| 3161 | CAGCAGGAAG CAACGAAGGG AAATGTCTTT CTTTTTCCTC |
| 3201 | TTAAGTTGTG GAGAGTGCAA CAGTAGCATA GGACCCTACC |
| 3241 | CTCTGGGCCA AGTCAAAGAC ATTCTGACAT CTTAGTATTT |
| 3281 | GCATATTCTT ATGTATGTGA AAGTTACAAA TTGCTTGAAA |
| 3321 | GAAAATATGC ATCTAATAAA AAACACCTTC TAAAATAA |

Fanconi anemia is a rare disorder with recessive inheritance and can lead to both birth defects and cancer. Most children with Fanconi Anemia (FA) manifest overt bone marrow failure (BMF) before the age of 10 years and often progress to myelodysplastic syndrome (MDS) or acute myelogenous leukemia (AML) despite best available therapy. For this reason, those with FA and BMF are considered for allogeneic hematopoietic stem cell (HSC) transplantation (allo-HSCT). However, suitable allo-HSCT donors cannot always be found and this approach risks strong immune suppression and graft versus host disease (GVHD).

The underlying problem in FA is bi-allelic inheritance of dysfunctional DNA repair genes (Fanconi complementation factors) that render HSCs fragile. The genes that are typically mutated in FA cases are the FANCA, FANCC, and FANCC genes. For example, an amino acid sequence for a FANCA Fanconi anemia group A protein isoform is available (NCBI accession no. NP_000126.2) and shown below as SEQ ID NO: 19.

| | |
|---|---|
| 1 | MSDSWVPNSA SGQDPGGRRR AWAELLAGRV KREKYNPERA |
| 41 | QKLKESAVRL LRSHQDLNAL LLEVEGPLCK KLSLSKVIDC |
| 61 | DSSEAYANHS SSFIGSALQD QASRLGVPVG ILSAGMVASS |
| 121 | VGQICTAPAE TSHPVLLTVE QRKKLSSLLE FAQYLLAHSM |
| 161 | FSRLSFCQEL WKIQSSLLLE AVWHLHVQGI VSLQELLESH |
| 201 | PDMHAVGSWL FRNLCCLCEQ MEASCQHADV ARAMLSDFVQ |
| 241 | MFVLRGFQKN SDLRRTVEPE KMPQVTVDVL QRMLIFALDA |
| 281 | LAAGVQEESS THKIVRCWFG VFSGHTLGSV ISTDPLKRFF |
| 321 | SHTLTQILTH SPVLKASDAV QMQREWSFAR THPLLTSLYR |
| 361 | RLFVMLSAEE LVGHLQEVLE TQEVHWQRVL SFVSALVVCF |
| 401 | PEAQQLLEDW VARLMAQAPE SCQLDSMVTA FLVVRQAALE |
| 441 | GPSAFLSYAD WFKASFGSTR GYHGCSKKAL VFLFTFLSEL |
| 481 | VPFESPRYLQ VHILHPPLVP GKYRSLLTDY ISLAKTRLAD |
| 521 | LKVSIENMGL YEDLSSAGDI TEPHSQALOD VEKAIMVFEH |
| 561 | TGNIPVTVME ASIFRRPYYV SHFLPALLTP RVLPKVPDSR |
| 601 | VAFIESLKRA DKIPPSLYST YCQACSAAEE KPEDAALGVR |
| 641 | AEPNSAEEPL GQLTAALGEL RASMTDPSQR DVISAQVAVI |
| 681 | SERLRAVLGH NEDDSSVEIS KIQLSINTPR LEPREHMAVD |
| 721 | LLLTSECQNL MAASSVAPPE RQGPWAALFV RTMCGRVLPA |
| 761 | VLTRLCQLLR HQGPSLSAPH VLGLAALAVH LGESRSALPE |
| 801 | VDVGPPAPGA GLPVPALFDS LLTCRTRDSL FFCLKFCTAA |
| 841 | ISYSLCKFSS QSRDTLCSCL SPGLIKKFQF LMFRLFSEAR |
| 881 | QPLSEEDVAS LSWRPLHLPS ADWQRAALSL WTHRTHREVL |
| 921 | KEEDVHLTYQ DWLHLELEIQ PEADALSDTE RQDPHQWAIH |
| 961 | EHFLPESSAS GGCDGDLQAA CTILVNALMD FHQSSRSYDH |
| 1001 | SENSDLVFGG RTGNEDIISR LQEMVADLEL QQDLIVPLGH |
| 1041 | TPSQEHFLFE IFRRRLQALT SGWSVAASLQ RQRELLMYKR |
| 1081 | ILLRLPSSVL CGSSFQAEQP ITARCEQFFH LVNSEMRNFC |
| 1121 | SHGGALTQDI TAHFFRGLLN ACLRSRDPSL MVDFILAKCQ |
| 1161 | TKCPLILTSA LVWWPSLEPV LLCRWRRHCQ SPLPRELQKL |
| 1201 | QEGRQFASDF LSPEAASPAP NPDWLSAAAL HFAIQQVREE |
| 1241 | NIRKQLKKLD CEREELLVFL FFFSLMGLLS SHLTSNSTTD |
| 1281 | LPKAFHVCAA ILECLEKRKI SWLALFOLTE SDLRLGRLLL |
| 1321 | RVAPDQHTRL LPFAFYSLLS YFHEDAAIRE EAFLHVAVDM |
| 1361 | YLKLVQLFVA GDTSTVSPPA GRSLELKGQG NPVELITKAR |
| 1401 | LFLLQLIPRC PKKSFSHVAE LLADRGDCDP EVSAALQSRQ |
| 1441 | QAAPDADLSQ EPHLF |

The gene encoding the FANCA Fanconi anemia group A protein isoform (SEQ ID NO: 19) resides on chromosome 16 (location 16q24.3; NC_000016.10 (89737549 . . . 89816647, complement). A nucleotide (cDNA) sequence that encodes the FANCA (SEQ ID NO: 19) polypeptide is shown below as SEQ ID NO:20.

| | |
|---|---|
| 1 | GAGCCGCCGC CGGGGCTGTA GGCGCCAAGG CCATGTCCGA |
| 41 | CTCGTGGGTC CCGAACTCCG CCTCGGGCCA GGACCCAGGG |
| 81 | GGCCGCCGGA GGGCCTGGGC CGAGCTGCTG GCGGGAAGGG |
| 121 | TCAAGAGGGA AAAATATAAT CCTGAAAGGG CACAGAAATT |
| 161 | AAAGGAATCA GCTGTGCGCC TCCTGCGAAG CCATCAGGAC |
| 201 | CTGAATGCCC TTTTGCTTGA GGTAGAAGGT CCACTGTGTA |
| 241 | AAAAATTGTC TCTCAGCAAA GTGATTGACT GTGACAGTTC |
| 281 | TGAGGCCTAT GCTAATCATT CTAGTTCATT TATAGGCTCT |
| 321 | GCTTTGCAGG ATCAAGCCTC AAGGCTGGGG GTTCCCGTGG |
| 361 | GTATTCTCTC AGCCGGGATG GTTGCCTCTA GCGTGGGACA |
| 401 | GATCTGCACG GCTCCAGCGG AGACGAGTCA CCCTGTGCTG |
| 441 | CTGACTGTGG AGCAGAGAAA GAAGCTGTCT TCCCTGTTAG |
| 481 | AGTTTGCTCA GTATTTATTG GCACACAGTA TGTTCTCCCG |
| 521 | TCTTTCCTTC TGTCAAGAAT TATGGAAAAT ACAGAGTTCT |
| 561 | TTGTTGCTTG AAGCGGTGTG GCATCTTCAC GTACAAGGCA |
| 601 | TTGTGAGGCT GCAAGAGCTG CTGGAAAGCC ATCCCGACAT |
| 641 | GCATGCTGTG GGATCGTGGC TCTTCAGGAA TCTGTGCTGC |
| 681 | CTTTGTGAAC AGATGGAAGC ATCCTGCCAG CATGCTGACG |
| 721 | TCGCCAGGGC CATGCTTTCT GATTTTGTTC AAATGTTTGT |
| 761 | TTTGAGGGGA TTTCAGAAAA ACTCAGATCT GAGAAGAACT |
| 801 | GTGGAGCCTG AAAAAATGCC GCAGGTCACG GTTGATGTAC |
| 841 | TGCAGAGAAT GCTGATTTTT GCACTTGACG CTTTGGCTGC |
| 881 | TGGAGTACAG GAGGAGTCCT CCACTCACAA GATCGTGAGG |
| 921 | TGCTGGTTCG GAGTGTTCAG TGGACACACG CTTGGCAGTG |
| 961 | TAATTTCCAC AGATCCTCTG AAGAGGTTCT TCAGTCATAC |
| 1001 | CCTGACTCAG ATACTCACTC ACAGCCCTGT GCTGAAAGCA |
| 1041 | TCTGATGCTG TTCAGATGCA GAGAGAGTGG AGCTTTGCGC |
| 1081 | GGACACACCC TCTGCTCACC TCACTGTACC GCAGGCTCTT |
| 1121 | TGTGATGCTG AGTGCAGAGG AGTTGGTTGG CCATTTGCAA |
| 1161 | GAAGTTCTGG AAACGCAGGA GGTTCACTGG CAGAGAGTGC |
| 1201 | TCTCCTTTGT GTCTGCCCTG GTTGTCTGCT TTCCAGAAGC |
| 1241 | GCAGCAGCTG CTTGAAGACT GGGTGGCGCG TTTGATGGCC |
| 1281 | CAGGCATTCG AGAGCTGCCA GCTGGACAGC ATGGTCACTG |
| 1321 | CGTTCCTGGT TGTGCGCCAG GCAGCACTGG AGGGCCCCTC |
| 1361 | TGCGTTCCTG TCATATGCAG ACTGGTTCAA GGCCTCCTTT |
| 1401 | GGGAGCACAC GAGGCTACCA TGGCTGCAGC AAGAAGGCCC |
| 1441 | TGGTCTTCCT GTTTACGTTC TTGTCAGAAC TCGTGCCTTT |
| 1481 | TGAGTCTCCC CGGTACCTGC AGGTGCACAT TCTCCACCCA |
| 1521 | CCCCTGGTTC CCGGCAAGTA CCGCTCCCTC CTCACAGACT |
| 1561 | ACATCTCATT GGCCAAGACA CGGCTGGCCG ACCTCAAGGT |
| 1601 | TTCTATAGAA AACATGGGAC TCTACGAGGA TTTGTCATCA |
| 1641 | GCTGGGGACA TTACTGAGCC CCACAGCCAA GCTCTTCAGG |
| 1681 | ATGTTGAAAA GGCCATCATG GTGTTTGAGC ATACGGGGAA |
| 1721 | CATCCCAGTC ACCGTCATGG AGGCCAGCAT ATTCAGGAGG |
| 1761 | CCTTACTACG TGTCCCACTT CCTCCCCGCC CTGCTCACAC |
| 1801 | CTCGAGTGCT CCCCAAAGTC CCTGACTCCC GTGTGGCGTT |
| 1841 | TATAGAGTCT CTGAAGAGAG CAGATAAAAT CCCCCCATCT |
| 1881 | CTGTACTCCA CCTACTGCCA GGCCTGCTCT GCTGCTGAAG |
| 1921 | AGAAGCCAGA AGATGCAGCC CTGGGAGTGA GGGCAGAACC |
| 1961 | CAACTCTGCT GAGGAGCCCC TGGGACAGCT CACAGCTGCA |
| 2001 | GTGGGAGAGC TGAGAGCCTC CATGACAGAC CCCAGCCAGC |
| 2041 | GTGATGTTAT ATCGGCACAG GTGGCAGTGA TTTCTGAAAG |
| 2081 | ACTGAGGGCT GTCCTGGGCC ACAATGAGGA TGACAGCAGC |
| 2121 | GTTGAGATAT CAAAGATTCA GCTCAGCATC AACACGCCGA |
| 2161 | GACTGGAGCC ACGGGAACAC ATGGCTGTGG ACCTCCTGCT |
| 2201 | GACGTCTTTC TGTCAGAACC TGATGGCTGC CTCCAGTGTC |
| 2241 | GCTCCCCCGG AGAGGCAGGG TCCCTGGGCT GCCCTCTTCG |
| 2281 | TGAGGACCAT GTGTGGACGT GTGCTCCCTG CAGTGCTCAC |
| 2321 | CCGGCTCTGC CAGCTGCTCC GTCACCAGGG CCCGAGCCTG |
| 2361 | AGTGCCCCAC ATGTGCTGGG GTTGGCTGCC CTGGCCGTGC |
| 2401 | ACCTGGGTGA GTCCAGGTCT GCGCTCCCAG AGGTGGATGT |
| 2441 | GGGTCCTCCT GCACCTGGTG CTGGCCTTCC TGTCCCTGCG |
| 2481 | CTCTTTGACA GCCTCCTGAC CTGTAGGACG AGGGATTCCT |
| 2521 | TGTTCTTCTG CCTGAAATTT TGTACAGCAG CAATTTCTTA |
| 2561 | CTCTCTCTGC AAGTTTTCTT CCCAGTCACG AGATACTTTG |
| 2601 | TGCAGCTGCT TATCTCCAGG CCTTATTAAA AAGTTTCAGT |
| 2641 | TCCTCATGTT CAGATTGTTC TCAGAGGCCC GACAGCCTCT |
| 2681 | TTCTGAGGAG GACGTAGCCA GCCTTTCCTG GAGACCCTTG |
| 2721 | CACCTTCCTT CTGCAGACTG GCAGAGAGCT GCCCTCTCTC |
| 2761 | TCTGGACACA CAGAACCTTC CGAGAGGTGT TGAAAGAGGA |
| 2801 | AGATGTTCAC TTAACTTACC AAGACTGGTT ACACCTGGAG |
| 2841 | CTGGAAATTC AACCTGAAGC TGATGCTCTT TCAGATACTG |
| 2881 | AACGGCAGGA CTTCCACCAG TGGGCGATCC ATGAGCACTT |
| 2921 | TCTCCCTGAG TCCTCGGCTT CAGGGGGCTG TGACGGAGAC |
| 2961 | CTGCAGGCTG CGTGTACCAT TCTTGTCAAC GCACTGATGG |
| 3001 | ATTTCCACCA AAGCTCAAGG AGTTATGACC ACTCAGAAAA |
| 3041 | TTCTGATTTG GTCTTTGGTG GCCGCACAGG AAATGAGGAT |
| 3081 | ATTATTTCCA GATTGCAGGA GATGGTAGCT GACCTGGAGC |
| 3121 | TGCAGCAAGA CCTCATAGTG CCTCTCGGCC ACACCCCTTC |
| 3161 | CCAGGAGCAC TTCCTCTTTG AGATTTTCCG CAGACGGCTC |
| 3201 | CAGGCTCTGA CAAGCGGGTG GAGCGTGGCT GCCAGCCTTC |
| 3241 | AGAGACAGAG GGAGCTGCTA ATGTACAAAC GGATCCTCCT |
| 3281 | CCGCCTGCCT TCGTCTGTCC TCTGCGGCAG CAGCTTCCAG |
| 3321 | GCAGAAGAGC CCATCACTGC CAGATGCGAG CAGTTCTTCC |
| 3361 | ACTTGGTCAA CTCTGAGATG AGAAACTTCT GCTCCCACGG |
| 3401 | AGGTGCCCTG ACACAGGACA TCACTGCCCA CTTCTTCAGG |
| 3441 | GGCCTCCTGA ACGCCTGTCT GCGGAGCAGA GACCCCTCCC |
| 3481 | TGATGGTGGA CTTCATACTG GCCAAGTGCC AGACGAAATG |
| 3521 | CGCCTTAATT TTGAGCTCTG CTCTGGTGTG GTGGCCGAGC |
| 3561 | CTGGAGCCTG TGCTGCTCTG CCGGTGGAGG AGACACTGCC |
| 3601 | AGAGCCCGCT GCCCCGGGAA CTGCAGAAGC TACAAGAAGG |
| 3641 | CCGGCAGTTT GCCAGCGATT TCCTCTCCCC TGAGGCTGCC |
| 3681 | TCCCCAGCAC CCAACCCGGA CTGGCTCTCA GCTGCTGCAC |
| 3721 | TGCACTTTGC GATTCAACAA GTCAGGGAAG AAAACATCAG |
| 3761 | GAAGCAGCTA AAGAAGCTGG ACTGCGAGAG AGAGGAGCTA |
| 3801 | TTGGTTTTCC TTTTCTTCTT CTCCTTGATG GGCCTGCTGT |
| 3841 | CGTCACATCT GACCTCAAAT AGCACCACAG ACCTGCCAAA |
| 3881 | GGCTTTCGAC GTTTGTGCAG CAATCCTCGA GTGTTTAGAG |
| 3921 | AAGAGGAAGA TATCCTGGCT GGCACTCTTT CAGTTGACAG |
| 3961 | AGAGTGACCT CAGGCTGGGG CGGCTCCTCC TCCGTGTGGC |
| 4001 | CCCGGATCAG CACACCAGGC TGCTGCCTTT CGCTTTTTAC |
| 4041 | AGTCTTCTCT CCTACTTCCA TGAAGACGCG GCCATCAGGG |
| 4081 | AAGAGGCCTT CCTGCATGTT GCTGTGGACA TGTACTTGAA |
| 4121 | GCTGGTCCAG CTCTTCGTGG CTGGGGATAC AAGCACAGTT |
| 4161 | TCACCTCCAG CTGGCAGGAG CCTGGAGCTC AAGGGTCAGG |
| 4201 | GCAACCCCGT GGAACTGATA ACAAAAGCTC GTCTTTTTCT |
| 4241 | GCTGCAGTTA ATAGCTCGGT GCCCGAAAAA GAGCTTCTCA |
| 4281 | CACGTGGCAG AGCTGCTGGC TGATCGTGGG GACTGCGACC |
| 4321 | CAGAGGTGAG CGCCGCCCTC CAGAGCAGAC AGCAGGCTGC |
| 4361 | CCCTGACGCT GACCTGTCCC AGGAGCCTCA TCTCTTCTGA |
| 4401 | CGGGACCTGC CACTGCACAC CAGCCCAGCT CCCGTGTAAA |
| 4441 | TAATTTATTA CAAGCATAAC ATGGAGCTCT TGTTGCACTA |
| 4481 | AAAAGTGGAT TACAAATCTC CTCGACTGCT TTAGTGGGGA |
| 4521 | AAGGAATCAA TTATTTATGA ACTGTCCGGC CCCGAGTCAC |
| 4561 | TCAGCGTTTG CGGGAAAATA AACCACTGGT CCCAGAGCAG |
| 4601 | AGGAAGGCTA CTTGAGCCGG ACACCAAGCC CGCCTCCAGC |
| 4641 | ACCAAGGGCG GGCAGCACCC TCCGACCCTC CCATGCGGGT |
| 4681 | GCACACGAAG GGTGAGGCTG ACACAGCCAC TGCGGAGTCC |
| 4721 | AGGCTGGTAG AGGTGCTCAT CCTCACTGCC GTCCTCAGGT |
| 4761 | GGGTTCGGGC TTCACCGCCT GGCCCTCTGT GGTCACAGAG |
| 4801 | GGGCTGGGTG GCCCAGGTGG TGGTTCCGCC TCCAGGGGCA |
| 4841 | GGGCCTTGTC CTGGGTCTGT GTCAGCGGGT GCACCATGGA |
| 4881 | CATGTGTACA TTGAGGTTGT GGGCCTTCTC AAACCGCCGG |
| 4921 | CCACACTGGT CACAGGCAAA GTCCAGCTCA GTCTCAGCCT |
| 4961 | TGTGTTTGGT CATGTGGTAC TTGAGGGATG CCCGCTGCCT |
| 5001 | GCACTGGAAC CCACAGACCT CACACCTGGG GGACAGAGGC |
| 5041 | AGATAAGAAG GTGCGAGGGC CACAGCCCTG GGAGGGGGTC |
| 5081 | CTGACTCACA CTTACTGCAA AGGCTTGGCT CCCGAATGTC |
| 5121 | GCATTTGGTG GACGAGAAGG TGCTTCCGCT GCTTGAAGGT |
| 5161 | TTGTCCACAT TCGTCACAGA TATAGTTCCG CACCTCTGAG |
| 5201 | AGGGGAGAGT CCAGTGAGTC CAGGCCCCTG ATGCTCCAAC |
| 5241 | CTCCCGGGGG GACGACGATG ACAATGTGAA ACCATCACAG |
| 5281 | CTGGGAAGAC ATTTCTGCAC ATGGTTCACC ATGCAGTGGG |
| 5321 | CCCAAGCAAG GGGCCTATGA GGGCCTCGTT TATTAAGATC |
| 5361 | TTTAAACTGC TTTATACACT GTCACGTGGC TTCA7CAGCT |
| 5401 | GTGTGCATTT CAGGATGGTT TTTAAAGAAA CCTCAGAAAG |
| 5441 | CTATTTCCTT AA |

In another example, an amino acid sequence for a FANCC Fanconi anemia group C protein isoform a is available (NCBI accession no. NP_000127.2) and shown below as SEQ ID NO:21.

| | |
|---|---|
| 1 | MAQDSVDLSC DYQFWMQKLS VWDQASTLET QQDTCLHVAQ |
| 41 | FQEFLRKMYE ALKEMDSNTV IERFPTIGQL LAKACWNPFI |
| 81 | LAYDESQKIL IWCLCCLINK EPQNSGQSKL NSWIQGVLSH |
| 121 | ILSALRFDKE VALFTQGLGY APIDYYPGLL KNMVLSLASE |
| 161 | LRENELNGFN TQRRMAPERV ASLSRVCVPL ITLTDVDPLV |
| 201 | EALLICHGRE PQEILQPEFF EAVNEAILLK KISLPMSAVV |
| 241 | CLWLRHLPSL EKAMLHLFEK LISSERNCLR RIECFIKDSS |
| 281 | LPQAACHPAI FRVVDEMFRC ALLETDGALE IIATIQVFTQ |
| 321 | CFVEALEKAS KQLRFALKTY FPYTSPSLAM VLLQDPQDIP |
| 361 | RGHWLQTLKH ISELLREAVE DQTHGSCGGP FESWFLFIHF |
| 401 | GGWAEMVAEQ LLMSAAEPPT ALLWLLAFYY GPRDGRQQRA |
| 441 | QTMVQVKAVL GHLLAMSRSS SLSAQDLQTV AGQGTDTDLR |
| 481 | APAQQLIRHL LLNFLLWAPG GHTIAWDVIT LMAHTAEITH |
| 521 | EIIGFLDQTL YRWNRLGIES PRSEKLAREL LKELRTQV |

The gene encoding the FANCC Fanconi anemia group C protein isoform a (SEQ ID NO:21) resides on chromosome 9 (location 9q22.32; NC_000009.12 (95099054 . . . 95317730, complement). A nucleotide (cDNA) sequence that encodes the FANCC (SEQ ID NO:21) polypeptide has NCBI accession no. NM_000136.3 is shown below as SEQ ID NO:22.

| | |
|---|---|
| 1 | AGAATGCACT GCTGACACGT GTGCGCGCGC GCGGCTCCAC |
| 41 | TGCCGGGCGA CCGCGGGAAA ATTCCAAAAA AACTCAAAAA |
| 81 | GCCAATACGA GGCAAAGCCA AATTTTCAAG CCACAGATCC |
| 121 | CGGGCGGTGG CTTCCTTTCC GCCACTGCCC AAACTGCTGA |
| 161 | AGCAGCTCCC GCGAGGACCA CCCGATTTAA TGTGTGCCGA |
| 201 | CCATTTCCTT CAGTGCTGGA CAGGCTGCTG TGAAGGGACA |
| 241 | TCACCTTTTC GCTTTTTCCA AGATGGCTCA AGATTCAGTA |
| 281 | GATCTTTCTT GTGATTATCA GTTTTGGATG CAGAAGCTTT |
| 321 | CTGTATGGGA TCAGGCTTCC ACTTTGGAAA CCCAGCAAGA |
| 361 | CACCTGTCTT CACGTGGCTC AGTTCCAGGA GTTCCTAAGG |
| 401 | AAGATGTATG AAGCCTTGAA AGAGATGGAT TCTAATACAG |
| 441 | TCATTGAAAG ATTCCCCACA ATTGGTCAAC TGTTGGCAAA |
| 481 | AGCTTGTTGG AATCCTTTTA TTTTAGCATA TGATGAAAGC |
| 521 | CAAAAAATTC TAATATGGTG CTTATGTTGT CTAATTAACA |
| 561 | AAGAACCACA GAATTCTGGA CAATCAAAAC TTAACTCCTG |
| 601 | GATACAGGGT GTATTATCTC ATATACTTTC AGCACTCAGA |
| 641 | TTTGATAAAG AAGTTGCTCT TTTCACTCAA GGTCTTGGGT |
| 681 | ATGCACCTAT AGATTACTAT CCTGGTTTGC TTAAAAATAT |
| 721 | GGTTTTATCA TTAGCGTCTG AACTCAGAGA GAATCATCTT |
| 761 | AATGGATTTA ACACTCAAAG GCGAATGGCT CCCGAGCGAG |
| 801 | TGGCGTCCCT GTCACGAGTT TGTGTCCCAC TTATTACCCT |
| 841 | GACAGATGIT GACCCCCTGG TGGAGGCTCT CCTCATCTGT |
| 881 | CATGGACGTG AACCTCAGGA AATCCTCCAG CCAGAGTTCT |
| 921 | TTGAGGCTGT AAACGAGGCC ATTTTGCTGA AGAAGATTTC |
| 961 | TCTCCCCATG TCAGCTGTAG TCTGCCTCTG GCTTCGGCAC |
| 1001 | CTTCCCAGCC TTGAAAAAGC AATGCTGCAT CTTTTTGAAA |
| 1041 | AGCTAATCTC CAGTGAGAGA AATTGTCTGA GAAGGATCGA |
| 1081 | ATGCTTTATA AAAGATTCAT CGCTGCCTCA AGCAGCCTGC |
| 1121 | CACCCTGCCA TATTCCGGGT TGTTGATGAG ATGTTCAGGT |
| 1161 | GTGCACTCCT GGAAACCGAT GGGGCCCTGG AAATCATAGC |
| 1201 | CACTATTGAG GTGTTTACGC AGTGCTTTGT AGAAGCTCTG |
| 1241 | GAGAAAGCAA GCAAGCAGCT GCGGTTTGCA CTCAAGACCT |
| 1281 | ACTTTCCTTA CACTTCTCCA TCTCTTGCCA TGGTGCTGCT |
| 1321 | GCAAGACCCT CAAGATATCC CTCGGGGACA CTGGCTCCAG |
| 1361 | ACACTGAAGC ATATTTCTGA ACTGCTCAGA GAAGCAGTTG |
| 1401 | AAGACCAGAC TCATGGGTCC TGCGGAGGTC CCTTTGAGAG |
| 1441 | CTGGTTCCTG TTCATTCACT TCGGAGGATG GGCTGAGATG |
| 1481 | GTGGCAGAGC AATTACTGAT GTCGGCAGCC GAACCCCCCA |
| 1521 | CGGCCCTGCT GTGGCTCTTG GCCTTCTACT ACGGCCCCCG |
| 1561 | TGATGGGAGG CAGCAGAGAG CACAGACTAT GGTCCAGGTG |
| 1601 | AAGGCCGTGC TGGGCCACCT CCTGGCAATG TCCAGAAGCA |
| 1641 | GCAGCCTCTC AGCCCAGGAC CTGCAGACGG TAGCAGGACA |
| 1681 | GGGCACAGAC ACAGACCTCA GAGCTCCTGC ACAACAGCTG |
| 1721 | ATCAGGCACC TTCTCCTCAA CTTCCTGCTC TGGGCTCCTG |
| 1761 | GAGGCCACAC GATCGCCTGG GATGTCATCA CCCTGATGGC |
| 1801 | TCACACTGCT GAGATAACTC ACGAGATCAT TGGCTTTCTT |
| 1841 | GACCAGACCT TGTACAGATG GAATCGTCTT GGCATTGAAA |
| 1881 | GCCCTAGATC AGAAAAACTG GCCCGAGAGC TCCTTAAAGA |
| 1921 | GCTGCGAACT CAAGTCTAGA AGGCACGCAG GCCGTGTGGG |
| 1961 | TGCCCGGCGT GAGGGATCAG GCTCGCCAGG GCCACAGGAC |
| 2001 | AGGTGATGAC CTGTGGCCAC GCATTTGTGG AGTAAGTGCC |
| 2041 | CTCGCTGGGC TGTGAGAATG AGCTGTACAC ATCTTGGGAC |
| 2081 | AATCTGCTAG TATCTATTTT ACAAAATGCA GAGCCAGGTC |
| 2121 | CCTCAGCCCA GACTCAGTCA GACATGTTCA CTAATGACTC |
| 2161 | AAGTGAGCCT TCGGTACTCC TGGTGCCCGC CCGGCCAGAC |
| 2201 | CGTCAGCTTG ATAATTACTA AAGCAAAGGC CTGGGTGGGA |
| 2241 | GAACAGGTTT CTAGTTTTTA CCCAAGTCAA GCTGCACATC |
| 2281 | TATTATTTAA AAATTCAAAG TCTTAGAACC AAGAATTTGG |
| 2321 | TCATGAACCA TTAAAGAATT TAGAGAGAAC TTAGCTCTTT |
| 2361 | TTAGACTCTT TTTAGGAGTC AGGGATCTGG GATAAAGCCA |
| 2401 | CACTGTCTTG CTGTATGGAG AAATTCTTCA AGGGGAGTCA |
| 2441 | GGGTCCCTCA GGCTTCCCTT GTGTCTCCCT GGACCTGCCT |
| 2481 | GACAGGCCAC AGGAGCAGAC AGCACACCCA AGCCCGGGCC |
| 2521 | TCCGGCACAC TCTTTCCACT CTGTATTTGC TAAATGATGC |
| 2561 | TAACTGCTAC CAAAAGGCCC TTGGGACATC AGAGGAGCCG |
| 2601 | GCAGGCGAAG GTAGAGGATG TGTTCCAGAA ACATTAGAAG |
| 2641 | GCAGGATTAA TTCAGTTAGT TAGTTCTCTT GTTAAATGGA |
| 2681 | AATGGGAATT GGAAATTCCT GATAAAGAAT TGGCCTGGCT |
| 2721 | GGGTGCAGTG GCTCACACCT GTGATCCCAG CACTTTGGGA |
| 2761 | GGCCAAGGCA GGGGGATTAC TTCAGCCCAG GAGTTCCAGA |
| 2801 | CTGCCTGGCT AACATGGCAA TACCCTATCT CTACTAAAAA |
| 2841 | TACAAAAATT ATCGGGGTGC AATGGCATGC ATCTGTAATC |
| 2881 | CCAGCTATTC AAGAGGCTGA GGCATGAGGA TCTCTTGAAC |
| 2921 | CCGGGAGGTG GGAGTTGTAG TGAGCCGAGA TCATGACACT |
| 2961 | GCACTCCAGC CTGGGCAACA GAGCGAGACC ATCTCTTAAA |
| 3001 | AAAAGGCATT GTTAGTGTAA TCTCAAGGTT AACATTTATT |
| 3041 | TCATGTCAGT ACAGGGTGCT TTTTCCTTTC AGGGACATTC |
| 3081 | TGGAATTGTA TTGGTTGTAC ATTCTTTTGT GTCTATTCTG |
| 3121 | TTTGTCAAGT GAGTCAAGAC TTGCTTTTGT CCATTTTGAT |
| 3161 | TTGTGTGTAT TAGTCTGAGT CTTGGCTCCG TTTTGAGGTA |
| 3201 | TGAGCAAAGT TTTGCTGGAT TAGAAGTTAA CCTTTAGGGA |
| 3241 | AATTCCTTAT TTTGGTATGT GGCAATGCTA ATAGATCCAC |
| 3281 | TGAAGATCTG GAAAATTCCA GGAACTTTTC ACCTGAGCCT |
| 3321 | TTCTTCTGAG AAATGCTGCA GTCAGAAGGG TGTGCTGGTA |
| 3361 | AAGTATTTTG GTGGCAGCTG CCATCATGGT CATTGCCTTC |
| 3401 | ATATAACATG CTTCGTGCTC ATGGTCATTG CCTTCATATA |
| 3441 | ACATGCTTCG TGCCATCATG ATCCTTGCCT TCATATAACA |
| 3481 | AACATGCTTC GTCAGAGGTG TTGGGGTTGA AAAAGGAGCT |
| 3521 | GCATGCTTCA CTGGAGTTGA GGGCCTCTCT CCTGTTCTGA |
| 3561 | CTTTAAGCCA GAACTTGTGG CTGGGCCATG GAAGCTGTGA |
| 3601 | CTCCTCTGTG GACATGGTGG CAGCAGGGAA CCCCTAGAGA |
| 3641 | GAGGGGCCAC TGGGACCAGG CCTCCTGTTG TGGAGGGACT |
| 3681 | CCTGGGACAG TCCTCCACCC TGTCCTGTGG TCCTGTGTAC |
| 3721 | AGGGTTGGCC TCTTCCTCCT CCCCTGCCAG GCCTCTGCCC |
| 3761 | ATGCCCCTTC CTTCCTTCTC CTGGGACTGG TGAAGCTAGG |
| 3801 | CATCTGGAAG ACTTCTTCCT AGCCTGGAAG CCCTGACCTC |
| 3841 | GGCCCATCTG CAGAATCTCC CAGTTCCTTC ACAGCTGCCG |
| 3881 | AGTCCTCTCA CGGGTGCGGT GGAGGCGGCC TTGCCGGTGG |
| 3921 | TGCTTTCTGG GCAGCCAGGG GTTCCTGGGT GGGAGGACTG |
| 3961 | TCCCTCTGGG GACGTGGCAC TGAAGTGCCT GCTGGCTTCA |
| 4001 | TGTGGCCCTT TGCCCTTTCC CAGCCTGAGA GATGCTCAAA |
| 4041 | GGTGGGGAGC TGGGGGAGCC ACCCCTCGGC CATTCCCTCC |
| 4081 | ACCTCCAAGA CAGGTGGCGG CCGGGCAGGC ACTCTTAAGC |
| 4121 | CCACCTCCCC CTCTTGTTGC CTTCGATTTC GGCAAAGCCT |
| 4161 | GGGCAGGTGG CACCGGGAAG GAATGGCATC CGAGATGCTG |
| 4201 | GGCGGGGACG CGGCGTGGCC GAGGGGGCCT TGACGGCGTT |
| 4241 | GGCGGGGCCT GGGCACAGGG GCAGCCGCAG GGAGGCAGGG |
| 4281 | ATGGCAAGGC GTGAAGCCAC CCTGGAAGGA AC!GGACCAA |
| 4321 | GGTCTTCAGA GGTGCGACAG GGTCTGGAAT CTGACCTTAC |
| 4361 | TCTAGCAGGA GTTTTTGTAG ACTCTCCCTG ATAGTXTAGT |
| 4401 | TTTTGATAAA GCATGCTGGT AAAACCACTA CCCTCAGAGA |
| 4441 | GAGCCAAAAA TACAGAAGAG GCGGAGAGCG CCCCTCCAAC |
| 4481 | CAGGCTGTTA TTCCCCTGGA CTCCGTGACA TCTGTGGAAT |
| 4521 | TTTTTAGCTC TTTAAAATCT GTAATTTGTT GTCTATTTTT |
| 4561 | TCATTCTAAA TAAAACTTCA GTTTGCACCT AA |

In another example, an amino acid sequence for a FANCG Fanconi anemia group protein is available (NCBI accession no. NP_004620.1) and shown below as SEQ ID NO:23.

| | |
|---|---|
| 1 | MSRQTTSVGS SCLDLWREKN DRLVRQAKVA QNSGLTLRRQ |
| 41 | QLAQDALEGL RGLLHSLQGL PAAVPVLPLE LTVTCNFIIL |
| 81 | RASLAQGFTE DQAQDIQRSL ERVLETQEQQ GPRLEQGLRE |
| 121 | LWDSVLRASC LLPELLSALH RLVGLQAALW LSADRLGDLA |
| 161 | LLLETLNGSQ SGASKDLLLL LKTWSPPAEE LDAPLTLQDA |
| 201 | QGLKDVLLTA FAYRQGLQEL ITGNPDKALS SLHEAASGLC |
| 241 | PRPVLVQVYT ALGSCHRKMG NPQRALLYLV AALKEGSAWG |
| 281 | PPLLEASRLY QQLGDTTAEL ESLELLVEAL NVPCSSKAPQ |
| 321 | ELIEVELLLP PPDLASPLHC GTQSQTKHIL ASRCLQTGRA |
| 361 | GDAAEHYLDL LALLLDSSEP RFSPPPSPPG PCMPEVFLEA |
| 401 | AVALIQAGRA QDALTLCEEL LSRTSSLLPK MSRLWEDARK |
| 441 | GTKELPYCPL WVSATHLLQG QAWVQLGAQK VAISEFSRCL |
| 481 | ELLFRATPEE KEQGAAFNCE QGCKSDAALQ QLRAAALISR |
| 521 | GLEWVASGQD TKALQDFLLS VQMCPGNRDT YFHLLQTLKR |
| 561 | LDRRDEATAL WWRLEAQIKG SHEDALWSLP LYLESYLSWI |
| 601 | RPSDRDAFLE EFRTSLPKSC DL |

The gene encoding the FANCG Fanconi anemia group G protein (SEQ ID NO:23) resides on chromosome 9 (location 9p13.3; NC_000009.12 (35073839 . . . 35079942, complement). A nucleotide (cDNA) sequence that encodes the FANCG (SEQ ID NO:23) polypeptide has NCBI accession no. NM_004629.2 is shown below as SEQ ID NO:24.

| | |
|---|---|
| 1 | CCTTTCICGA GGCTGTGGCC TCCGCGAGAG CCGAGCGGGC |
| 41 | CGCACCGCCG GCCGTGCGAC TGCCCCAGTC AGACACGACC |
| 81 | CCGGCTTCTA GCCCGCCTAA GCCTGTTTGG GGTTGCTGAC |
| 121 | TCGTTTCCTC CCCGAGTTTC CCGCGGGAAC TAACTCTTCA |
| 161 | AGAGGACCAA CCGCAGCCCA GAGCTTCGCA GACCCGGCCA |
| 201 | ACCAGAGGCG AGGTTGAGAG CCCGGCGGGC CGCGGGGAGA |
| 241 | GAGCGTCCCA TCTGTCCTGG AAAGCCTGGG CGGGTGGATT |
| 281 | GGGACCCCGA GAGAAGCAGG GGAGCTCGGC GGGGTGCAGA |
| 321 | AGTGCCCAGG CCCCTCCCCG CTGGGGTTGG GAGCTTGGGC |
| 361 | AGGCCAGCTT CACCCTTCCT AAGTCCGCTT CTGGTCTCCG |
| 401 | GGCCCAGCCT CGGCCACCAT GTCCCGCCAG ACCACCTCTG |
| 441 | TGGGCTCCAG CTGCCTGGAC CTGTGGAGGG AAAAGAATGA |
| 481 | CCGGCTCGTT CGACAGGCCA AGGTGGCTCA GAACTCCGGT |
| 521 | CTGACTCTGA GGCGACAGCA GTTGGCTCAG GATGCACTGG |
| 561 | AAGGGCTCAG AGGGCTCCTC CATAGTCTCC AAGGGCTCCC |
| 601 | TGCAGCTGTT CCTGTTCTTC CCTTGGAGCT GACTGTCACC |
| 641 | TGCAACTTCA TTATCCTGAG GGCAAGCTTG GCCCAGGGTT |
| 681 | TCACAGAGGA TCAGGCCCAG GATATCCAGC GGAGCCTAGA |
| 721 | GAGAGTGCTG GAGACACAGG AGCAGCAGGG GCCCAGGTTG |
| 761 | GAACAGGGGC TCAGGGAGCT GTGGGACTCT GTCCTTCGTG |
| 801 | CTTCCTGCCT TCTGCCGGAG CTGCTGTCTG CCCTGCACCG |
| 841 | CCTGGTTGGC CTGCAGGCTG CCCTCTGGTT GAGTGCTGAC |
| 881 | CGTCTTGGGG ACCTGGCCTT GTTACTAGAG ACCCTGAATG |
| 921 | GCAGCCAGAG TGGAGCCTCT AAGGATCTGC TGTTACTTCT |
| 961 | GAAAACTTGG AGTCCCCCAG CTGAGGAATT AGATGCTCCA |
| 1001 | TTGACCCTGC AGGATGCCCA GGGATTGAAG GATGTCCTCC |
| 1041 | TGACAGCATT TGCCTACCGC CAAGGTCTCC AGGAGCTGAT |
| 1081 | CACAGGGAAC CCAGACAAGG CACTAAGCAG CCTTCATGAA |
| 1121 | GCGGCCTCAG GCCTGTGTCC ACGGCCTGTG TTGGTCCAGG |
| 1161 | TGTACACAGC ACTGGGGTCC TGTCACCGTA AGATGGGAAA |
| 1201 | TCCACAGAGA GCACTGTTGT ACTTGGTTGC AGCCCTGAAA |
| 1241 | GAGGGATCAG CCTGGGGTCC TCCACTTCTG GAGGCCTCTA |
| 1281 | GGCTCTATCA GCAACTGGGG GACACAACAG CAGAGCTGGA |
| 1321 | GAGTCTGGAG CTGCTAGTTG AGGCCTTGAA TGTCCCATGC |
| 1361 | AGTTCCAAAG CCCCGCAGTT TCTCATTGAG GTAGAATTAC |
| 1401 | TACTGCCACC ACCTGACCTA GCCTCACCCC TTCATTGTGG |
| 1441 | CACTCAGAGC CAGACCAAGC ACATACTAGC AAGCAGGTGC |
| 1481 | CTACAGACGG GGAGGGCAGG AGACGCTGCA GAGCATTACT |
| 1521 | TGGACCTGCT GGCCCTGTTG CTGGATAGCT CGGAGCCAAG |
| 1561 | GTTCTCCCCA CCCCCCTCCC CTCCAGGGCC ctgtatgcct |
| 1601 | GAGGTGTTTT TGGAGGCAGC GGTAGCACTG ATCCAGGCAG |
| 1641 | GCAGAGCCCA AGATGCCTTG ACTCTATGTG AGGAGTTGCT |
| 1681 | CAGCCGCACA TCATCTCTGC TACCCAAGAT GTCCCGGCTG |
| 1721 | TGGGAAGATG CCAGAAAAGG AACCAAGGAA CTGCCATACT |
| 1761 | GCCCACTCTG GGTCTCTGCC ACCCACCTGC TTCAGGGCCA |
| 1801 | GGCCTGGGTT CAACTGGGTG CCCAAAAAGT GGCAATTAGT |
| 1841 | GAATTTAGCA GGTGCCTCGA GCTGCTCTTC CGGGCCACAC |
| 1881 | CTGAGGAAAA AGAACAAGGG GCAGCTTTCA ACTGTGAGCA |
| 1921 | GGGATGTAAG TCAGATGCGG CACTGCAGCA GCTTCGGGCA |
| 1961 | GCCGCCCTAA TTAGTCGTGG ACTGGAATGG GTAGCCAGCG |
| 2001 | GCCAGGATAC CAAAGCCTTA CAGGACTTCC TCCTCAGTGT |
| 2041 | GCAGATGTGC CCAGGTAATC GAGACACTTA CTTTCACCTG |
| 2081 | CTTCAGACTC TGAAGAGGCT AGATCGGAGG GATGAGGCCA |
| 2121 | CTGCACTCIG GTGGAGGCTG GAGGCCCAAA CTAAGGGGTC |
| 2161 | ACATGAAGAT GCTCTGTGGT CTCTCCCCCT GTACCTAGAA |
| 2201 | AGCTATTTGA GCTGGATCCG TCCCTCTGAT CGTGACGCCT |
| 2241 | TCCTTGAAGA ATTTCGGACA TCTCTGCCAA AGTCTTGTGA |
| 2281 | CCTGTAGCTG CCACGTTTTG AAGAGCTTGA GCTGGGTCCC |
| 2321 | CAGTGGGCTG TCTCTCTGTG GGGAGGGCTT TCTGCTTCAC |
| 2361 | CATCATTAGG AATGTGACCA TTCCTATATA ATTCCTGGAC |
| 2401 | TGGTGAGATT GGTGGTAGGC CTGTGAAATT TGCCCTAGTT |
| 2441 | ACTAGCATTC TCGTTTTGGA GGAAACAATC TCTGCCACCA |
| 2481 | CCAAGTCATT GACTTTGCTC GAGGCACCTT TTTTCCTGTT |
| 2521 | TCTCCTTTTC TGTTGTCGAG TAAAATTTCA TATTTA |

Restoration of the normal Fanconi complementation group factor rescues this fragility, making gene therapy with engineered autologous HSCs possible for FA. But this is difficult now because patients with FA have vastly reduced HSCs and those HSCs that can be harvested are hard to maintain during genetic manipulation. These limitations not only represent barriers to safe clinical gene therapy but the paucity of human FA HSCs available for research and development also limits opportunities to develop new therapeutic approaches. As illustrated in the Examples, such problems can be solved using the methods described herein.

In some cases, toxicity associated with chemotherapy can be treated by repairing or altering the sequence of folate pathway genes. Such folate pathway genes and sequence variations therein can influence plasma concentrations of methotrexate (MTX). Hence, MTX-induced toxicity in subjects can be treated by modifying one or more folate pathway genes in hematopoietic stem and progenitor cells, then administering the modified hematopoietic stem and progenitor cells after they were expanded. Examples of folate pathway genes that can be repaired, altered or modified include solute carrier family 19, member1 (SLC19A1), methylenetetrahydrofolate reductase (MTHFR) and 5-aminoimidazole-4-carboxamide ribonucleotide formyltransferase (ATIC). These genes and other genes can be modified in the hematopoietic stem and progenitor cells prior to expansion.

The methods described herein can therefore be used to repair or modify these and other types of genetic defects in other genes. Hematopoietic stem and progenitor cells having genetic defects can be collected, and one or more genetic defects can be repaired or modified in the one or more collected hematopoietic stem and progenitor cells to generate one or more repaired/modified hematopoietic stem and progenitor cells. As illustrated herein, although it is easier to expand a pool of cells (e.g., 10-5000), a single cell or just a few cells can be expanded into therapeutically useful numbers of cells. Hence, one or more repaired/modified hematopoietic stem and progenitor cells can be selected for expansion. After about 5-14 days, a population of sufficient numbers of repaired or modified hematopoietic stem and progenitor cells is available for administration to a recipient subject. In some cases, the recipient subject can be the original donor of the hematopoietic stem and progenitor cells.

The screening methods described herein can also identify agents that are uniquely capable of treating the types of diseases or conditions that a subject may suffer from. In some cases, agents may be identified that are useful for depleting or eliminating undesirable cells (e.g., cancer or diseased cells) from patients, include the cell donor. For example, donors can have a disease or condition resulting from a genetic defect. In some cases, the donors may not know that they have any such genetic defects (e.g., neoplastic stem cells). In other cases, a patient may be aware that they have a disease or condition but the patient may not know what cell type is correlated with the disease or condition.

### Screening Methods

Also described herein are screening methods that can identify whether collected hematopoietic stem and progenitor cells contain undesirable cell types (e.g. cancer stem cells) and screening methods that can identify agents useful as therapeutic agents for treatment of a variety of diseases and conditions.

Cells obtained from a donor can be screened for mutations and undesirable cell types. Each patient's collection of hematopoietic stem and progenitor cells may require analysis and identification of genetic defects. The methods described herein can facilitate expansion of donor cells so that sufficient cells are present to allow such screening.

The methods described herein can also be used to identify appropriate therapeutic regiments and agents for treatment of known as well as poorly understood diseases and conditions. Such a method can involve introducing one or more different test agents into different receptacles that contain aliquots of hematopoietic stem and progenitor cells to generate a series of test mixtures. The hematopoietic stem and progenitor cells employed in the test mixtures can be from a donor who has a disease or condition. Some of the cells in the original hematopoietic stem and progenitor cell sample can be diseased, while others may not be. The test mixtures can be incubated for a time and under conditions sufficient to expand the cells in the test mixtures. The growth of diseased cells in the test mixtures can be compared to the growth of the un-diseased cells in the same receptable. Hence, each receptacle has its own internal control. In some cases, control receptacles that contain aliquots of the of hematopoietic stem and progenitor cells but without the test agent(s) can also be used. Test agents that reduce the growth or survival of undesirable cells compared to control cells can be selected. Such selected test agents can be used, or can be further evaluated for use, as therapeutic agents for treatment of a disease or condition associated with the diseased cells in the collected hematopoietic stem and progenitor cells.

A screening method was developed for identifying agents useful for depleting and eliminating neoplastic cells from a population of hematopoietic stem cells collected from a patient or donor. The screen is useful, for example, when collecting stem cells from the bone marrow or peripheral blood cells of patients suffering from a disease or condition (e.g., cancer patients). Simply reintroducing such patient stem cells back into the patients after the patients have undergone treatment (e.g., radiation treatment or chemotherapy) can reintroduce the neoplastic stem cells that will later re-populate the hematological cells that differentiate from the collected stem cells. However, when the collected hematopoietic stem and progenitor cells are treated to reduce or eliminate the neoplastic cells, the treated hematopoietic stem and progenitor cells can then be safely administered to a patient. For example, hematopoietic stem and progenitor cells can be sorted and healthy, non-cancerous cells can be selected for expansion.

### Administration of Expanded Cells

Expanded hematopoietic stem and progenitor cells generated as described herein can be employed for regeneration and engraftment in a human patient or other subjects in need of such treatment. The cells are administered in a manner that permits them to graft and reconstitute or regenerate within a subject or recipient. Devices are available that can be adapted for administering cells, for example, intravascularly.

Expanded hematopoietic stem and progenitor cells can be administered to by systemic injection. For example, the cells can be administered intravascularly. In some embodiments, the cells can be administered parenterally by injection into a blood vessel or into a convenient cavity.

Many cell types are capable of migrating to an appropriate site for regeneration and differentiation within a subject. To determine the suitability of cell compositions for therapeutic administration, the hematopoietic stem and progenitor cells can first be tested in a suitable animal model (e.g., a mouse, rat or other animal). At one level, the expanded hematopoietic stem and progenitor cells are assessed for their ability to survive and maintain their phenotype in vivo. Cells can also be assessed to ascertain whether they populate a substantial percentage of hematological cells in vivo, or to determine an appropriate number of cells to be administered. Cell compositions can be administered to immunodeficient animals (such as nude mice, or animals rendered immunodeficient chemically or by irradiation). Blood and/or bone marrow can be harvested after a period of time and assessed to ascertain if the administered cells or progeny thereof are still present, are alive, and/or have migrated to desired or undesired locations.

An expanded population of hematopoietic stem and progenitor cells can be introduced by injection, catheter, implantable device, or the like. A population of expanded cells can be administered in any physiologically acceptable excipient or carrier that does not adversely affect the cells.

A population of expanded hematopoietic stem and progenitor cells can be supplied in the form of a pharmaceutical composition. Such a composition can include an isotonic excipient prepared under sufficiently sterile conditions for human administration. For general principles in medicinal formulation, the reader is referred to Cell Therapy: Stem Cell Transplantation, Gene Therapy. and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The choice of the cellular excipient and any accompanying constituents of the composition that includes a population of expanded cells can be adapted to optimize administration by the route and/or device employed.

A composition that includes a population of expanded hematopoietic stem and progenitor cells can also include or be accompanied by one or more other ingredients that facilitate engraftment or functional mobilization of the expanded cells.

The population of expanded hematopoietic stem and progenitor cells generated by the methods described herein can include some percentage of non-stem cells or non-progenitor cells. For example, a population of expanded cells for use in compositions and for administration to subjects can contain endothelial cells. The presence of such endothelial cells has no adverse effects, and in some cases can actually be helpful.

However, a population of expanded hematopoietic stem and progenitor cells for use in compositions and for administration to subjects can have less than about 90% non-stem cells or non-progenitor cells, less than 80% non-stem cells or non-progenitor cells, less than 70% non-stem cells or non-progenitor cells, less than about 60% non-stem cells or non-progenitor cells, less than about 50% non-stem cells or non-progenitor cells, less than about 40% non-stem cells or non-progenitor cells, less than about 30% non-stem cells or non-progenitor cells, less than about 25% non-stem cells or non-progenitor cells, less than about 20% non-stem cells or non-progenitor cells, less than about 15% non-stem cells or non-progenitor cells, less than about 12% non-stem cells or non-progenitor cells, less than about 10% non-stem cells or non-progenitor cells, less than about 8% non-stem cells or non-progenitor cells, less than about 6% non-stem cells or non-progenitor cells, less than about 5% non-stem cells or non-progenitor cells, less than about 4% non-stem cells or non-progenitor cells, less than about 3% non-stem cells or non-progenitor cells, less than about 2% non-stem cells or non-progenitor cells, or less than about 1% non-stem cells or non-progenitor cells of the total cells in the cell population.

The number of cells administered to a subject or a patient can vary. For example, subjects with different diseases and/or conditions can need different amounts of hematopoietic stem and progenitor cells. In some cases, number of hematopoietic stem and progenitor cells in the cell compositions described herein can be packaged for ready administration to a subject or patient. For example, the cells can be packaged to contain at least 1 million cells, or at least 5 million cells, at least 10 million cells, or at least 25 million cells, at least 50 million cells, or at least 70 million cells, at least 100 million cells, or at least 200 million cells, at least 300 million cells, at least 400 million cells, at least 500 million cells, or at least 600 million cells, at least 700 million cells, at least 800 million cells, at least 1000 million cells, or at least 2000 million cells, at least 5000 million cells, at least 7000 million cells, at least 10,000 million cells, or at least 30,000 million cells, at least 50,000 million cells, or at least 100,000 million cells.

### Definitions

As used herein, the term “treating” and “treatment” refers to administering to a subject an effective amount of a composition so that the recipient has a reduction in at least one symptom of the disease or an improvement in the disease, for example, beneficial or desired therapeutic or clinical results. For purposes of this invention, beneficial or desired therapeutic or clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether empirically detectable or undetectable. Treating can refer to prolonging survival as compared to expected survival if not receiving treatment. Thus, one of skill in the art realizes that a treatment may improve the disease condition but may not be a complete cure for the disease. The term “treatment” includes prophylaxis. Treatment is “effective” if the progression of a disease is reduced or halted. “Treatment” can also mean prolonging survival as compared to expected survival if not receiving treatment. Those in need of treatment include those already diagnosed with a condition (e.g., a liver condition such as chronic liver failure), as well as those likely to develop a condition due to genetic susceptibility or other factors such as alcohol consumption, diet, toxic exposure, and health.

Where an individual is to be treated with hematopoietic stem and progenitor cells, the individual's own hematopoietic stem and progenitor cells can be used, for example after expansion according to the methods described herein.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals with a hematological or immunological disease, condition, or disorder, and individuals with hematological disorder-related or immunological disorder-related characteristics or symptoms.

The following non-limiting Examples illustrate some of the experimental work involved in developing the invention.

### Example 1: Materials and Methods

This Example describes some of the materials and methods used in the development of the invention.

### Endothelial Cells and Adult Human HSPCs.

Primary human umbilical vein endothelial cell (EC) vascular niche cells were generated by lentiviral (LV) expression of adenoviral E4ORF1 using methods described by Seandel et al. (Proc Natl Acad Sci USA 105, 19288-19293 (2008)). Adult human hematopoietic stem and progenitor cells (aHSPCs) were obtained from bone marrow or by mobilization of peripheral blood (PB) using procedures and protocols approved by the Weill Cornell Medicine and/or Memorial Sloan Kettering Cancer Center Institutional Review Board. Mobilized peripheral blood CD34+ aHSPCs were purified using CliniMACs immunomagnetic bead (Miltenyi Biotec) separation and clinical grade processing for hematopoietic stem cell transplant (HSCT). After CD34-enrichment, mobilized aHSPCs were cryopreserved using standard banking procedures. Upon release of the mobilized donor units for research, samples were thawed, aliquoted and re-cryopreserved before use. Cells from patients with sickle cell disease (SCD) were mobilized using single agent plerixafor whereas cells from healthy HSCT donors were mobilized using GCSF or GCSF with plerixafor. Marrow samples were collected at the time of bone marrow harvest of healthy donors and CD34+ cells were purified using immunomagnetic beads (Miltenyi Biotec) and either used fresh or were cryopreserved.

Ex Vivo aHSPC Expansion.

Vascular niche platform ECs were plated at about confluence 12 to 48 hours prior to initiation of co-culture in M199 medium containing 10% GMP fetal bovine serum (FBS), FGF2, EGF. IGF (all at 10 ng/mL). Prior to plating, aHSPCs, medium was removed from the vascular niche, the cells were gently washed with PBS and then medium was replaced with StemSpan (STEMCELL Technologies) supplemented with 50 ng/mL each of KITL, THPO and FLT3L (PeproTech). The same medium and procedures were used when aHSPCs were cultured with cytokines alone (without the vascular niche).

The following additives were included in some of the culture media employed during various experiments described herein: SR1 (BioVision Technologies), UM171 (BioVision Technologies), N-Acetylcysteine (NAC) (Pfizer). BAY 87-2243 (Selleck Chem), IOX2 (Selleck Chem), CXCL12 (PeproTech), BMP4 (PeproTech), Plerixafor (PeproTech), Noggin (Thermo), or combinations thereof.

Cultures were maintained at various oxygen tension levels (as indicated for the experiments described herein) for seven days. If the oxygen tension is not indicated, then normal atmospheric levels of oxygen were used. Cells were harvested after brief digestion with TrypLE® (Thermofisher) and were maintained on ice unless otherwise indicated.

Cumulative expansion of CD34+ cells was assessed by comparing the quantified number of functional and/or immunophenotypic aHSPC populations prior to expansion to the quantified number of aHSPC populations after expansion. Expanded cells were analyzed by FACS, colony forming cell (CFC) assays, and in vivo engraftment into immunodeficient (NSG) mice.

### Flow Cytometry and Cell Sorting

FACS analyses were performed using a LSRII/SORP or other instrument (BD Bioscience) with the following antibodies from BioLegend: human AF700-CD45, PerCP/Cy5.5-CD31, PE/Cy7-CD34, APC-CD38, APC/Cy7-CD45RA, PE-CD90, FITC-CD14, FITC-CD15, APC/Cy7-CD19, APC/Cy7-CD3 and mouse PE-CD45. Flow cytometry of unexpanded and expanded aHSPCs was performed with the indicated antibodies and cells were sorted using Influx sorter (BD Biosciences).

### Transplantation of Immune Deficient Mice

All mice experiments were approved by the Weill Cornell Medical College Institutional Animal Care and Use committee (IACUC). Five week-old NOD.Cg-Prkdc^{scid}Il2rg^{tm1Wjl}/SzJ (NSG) mice were sub-lethally irradiated (250 cGy) and cell transplantations were performed by tail vein injection using various expanded or unexpanded aHSPCs. Engraftment was assessed at least 16 weeks after transplantation. Murine engraftment was evaluated using the various antibodies including murine and human CD45 (mCD45 and hCD45, respectively) and Hoechst to gate on nucleated cells. Human cells were sorted by FACS or immunomagnetic beads from murine marrow for subsequent analysis.

### Colony Forming Cell Assays for Myeloid Progenitors.

Expanded (1000 cells) or unexpanded (500 cells) were plated in Method-Cult H4034 Stem Cell Technologies Inc.) and cultures were maintained in humidified CO₂ incubator at ambient oxygen for 14 days prior to analysis. Following culture, 60 to 80 colonies were identified and classified per 35 mm dish using an inverted microscope (Zeiss).

### Analysis of Reactive Oxygen Species (ROS), Lipid Peroxidation, and Intracellular Antigens

Analysis of intracellular ROS was performed by flow cytometry using the CellROX Green Reagent (Life Technologies). Briefly, cultured aHSPCs were selected as indicated and harvested as previously described. Harvested cells were stained with 5 μM CellROX Green for 30 min on ice to provide surface staining for immunophenotyping. Cells were then washed and analyzed by flow cytometry. Lipid peroxidation was measured using fluorogenic linoleamide alkyne CLICK chemistry reagent (Click-iT LAA. Thermo). Adult-Derived-HSPCs were cultured without (NoEC) or with endothelial cells (EC) at different oxygen tensions. After 4 days culture, cultures were exposed to LAA for 30 minutes prior to harvest and analysis. Following culture and harvest, cells were surface immunostained, washed and then fixed and permeabilized, followed by intracellular staining for HIF1α or γH2AX. Cells were washed and analyzed by flow cytometry.

### Lentiviral Vector Production, Titration, and Vector Copy Number Quantification

Purified vectors (Globine lentiviral vectors (LV), as well as various batches of PGK.GFP, PGK.mCherry lentiviral vectors) were produced and titered using GMP compliant (cGMP) processes. Briefly, lentiviral vectors were quantified by titration using HeLa cells. Titered cells were maintained in culture for at least 2 weeks prior to assessing vector copy number (VCN). At least 4 dilutions were used and curves were fit to VCN vs dilution to determine viral titer by least squares optimization. Vector copy number was also measured using an RRE amplicon or LV-specific element and RPP30 in BioRad QX200 droplet digital PCR (ddPCR) machine with automated droplet generator.

Analysis of Engrafted aHSPC Hemoglobin Expression in NSG Mouse Recipients

Human CD34+ cells were isolated from marrow of engrafted NSG mice using immunomagnetic beads (Miltenyi). Purified cells were cultured to support differentiation to towards erythrocytes. At the time of harvest, differentiated erythroid cells were characterized by flow cytometry to ensure adequate differentiation and harvested by centrifugation at 500 g×5 minutes prior to lysis in sterile water. Supernatant containing hemoglobin was analyzed in capillary electrophoresis system using clinical protocols. Red blood cells (RBCs) from healthy normal donors were used to aid calibration of peaks as per standard operating procedures at WCM for clinical hemoglobin electrophoresis.

### Example 2: Expansion of Functional Adult Human Hematopoietic Stem and Progenitor Cells (HSPCs) Using Endothelial Cells and Hypoxia

Efforts to replace niche activities ex vivo using small molecule agents SR1 and UM171 have shown some promise for their ability to expand human umbilical cord blood (CB) CD34+ HSPCs (CB-HSPCs) (Boitano et al. Science 329, 1345-1348 (2010); Fares et al., Science 345, 1509-1512 (2014)). But expansion of adult donor CD34+ HSPCs from marrow or from mobilized blood (AD-HSPCs) has proven to be a greater challenge. Adult-Derived-HSPCs capable of long-term multilineage engraftment of immune deficient mice are lost during propagation with the Aryl Hydrocarbon Receptor (AhR) antagonist Stemregenin 1 (SR1) (Zonari et al. Stem Cell Reports 8, 977-990 (2017); Gu et al. Hum Gene Ther Methods 25, 221-231 (2014)). Concerns also linger over possible ill effects of targeting aryl-hydrocarbon receptors in hematopoietic stem cells (HSCs) (Boitano et al. Science 329, 1345-1348 (2010); Chou et al. Cell stem cell 7, 427428 (2010); Singh et al. Stem cells and development 20, 769-784 (2011); Singh et al. Stem cells and development 23, 95-106 (2014); Gasiewicz et al. Annals of the New York Academy of Sciences (2014)). The pyrimidoindole derivative UM171 (Fares et al. Science 345, 1509-1512 (2014)) acts independently of AhR to robustly expand CB-HSPCs ex vivo but its effects on AD-HSPCs appear modest (Zonari et al. Stem Cell Reports 8, 977-990 (2017)).

Methods were evaluated for identifying conditions that would allow ex vivo expansion of adult hematopoietic stem and progenitor cells (AD-HSPCs).

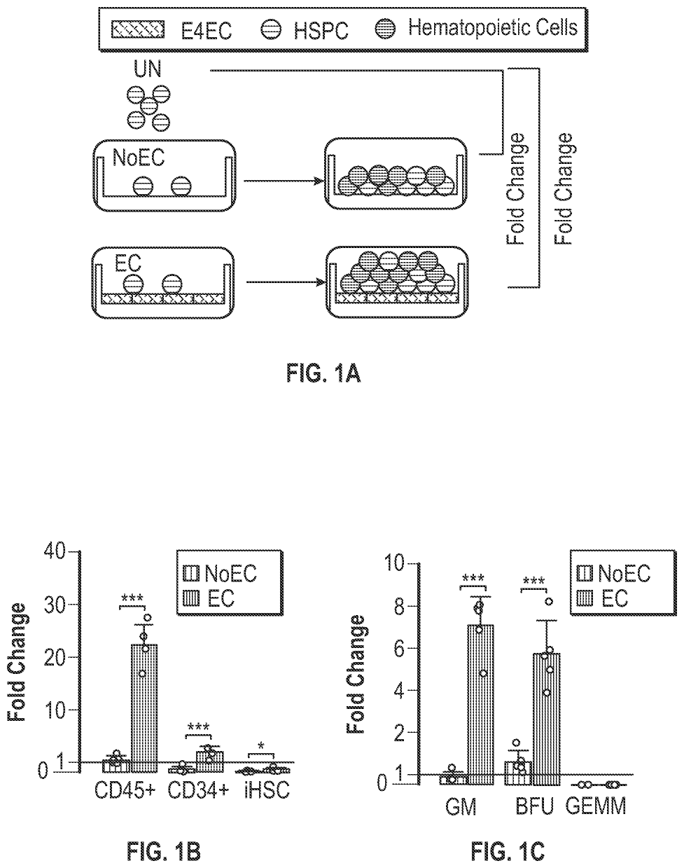

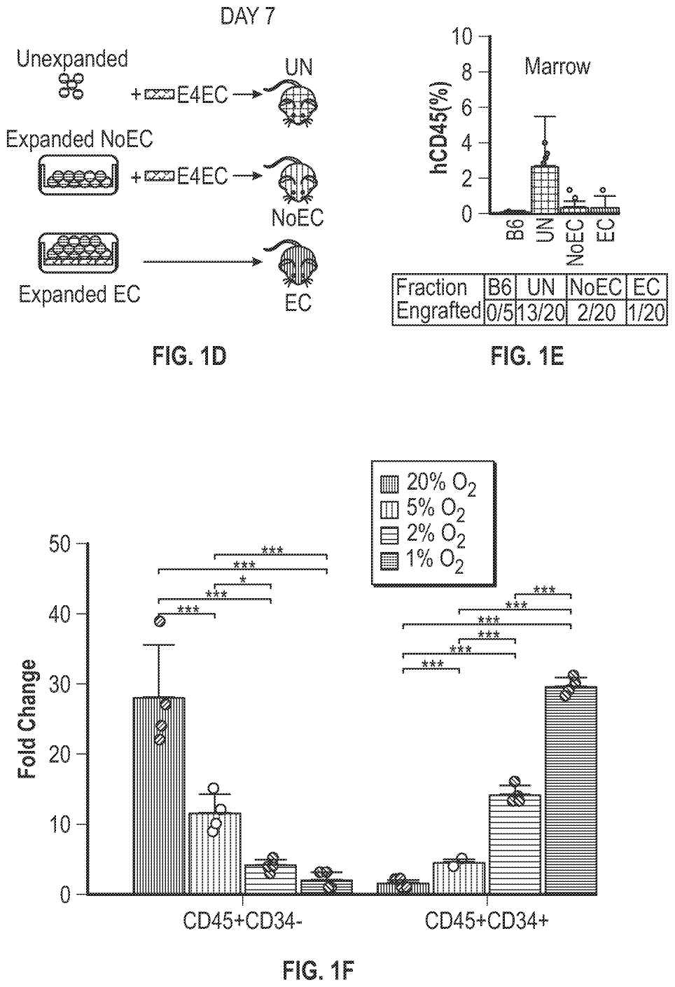

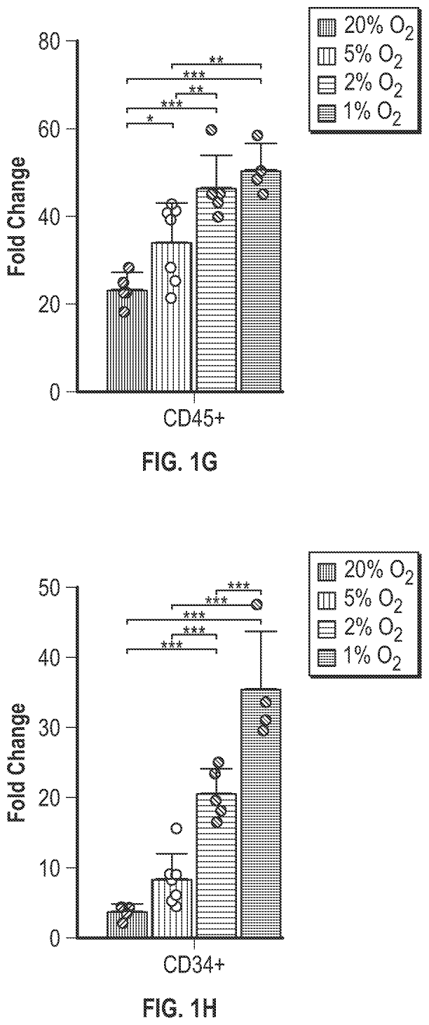

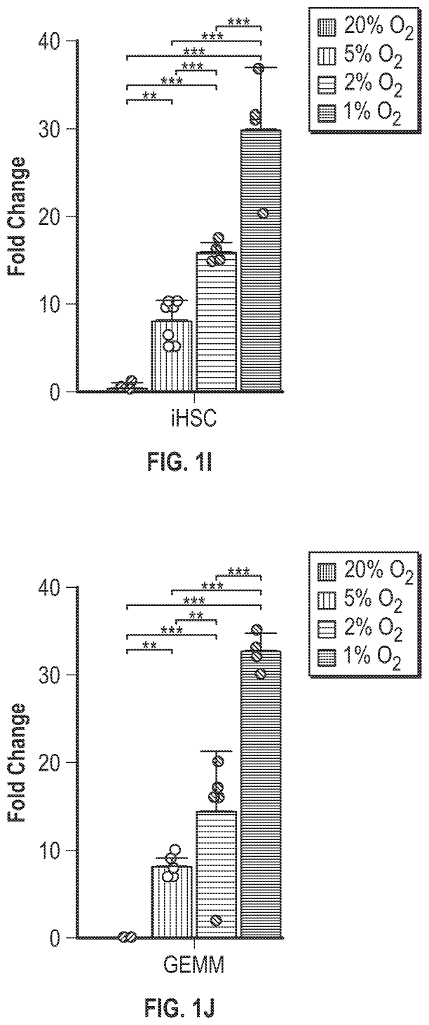

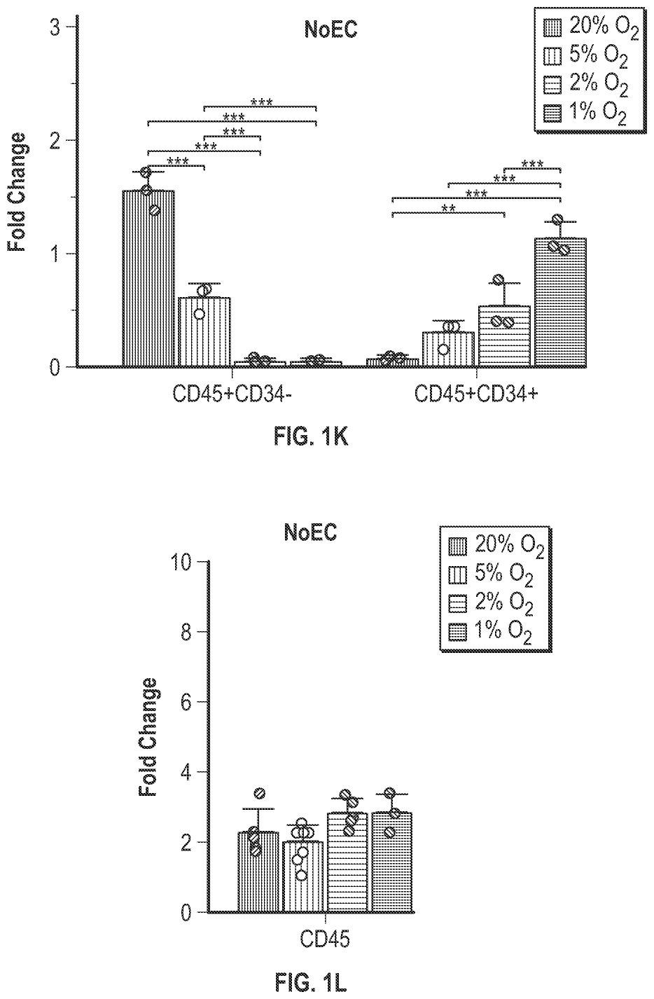

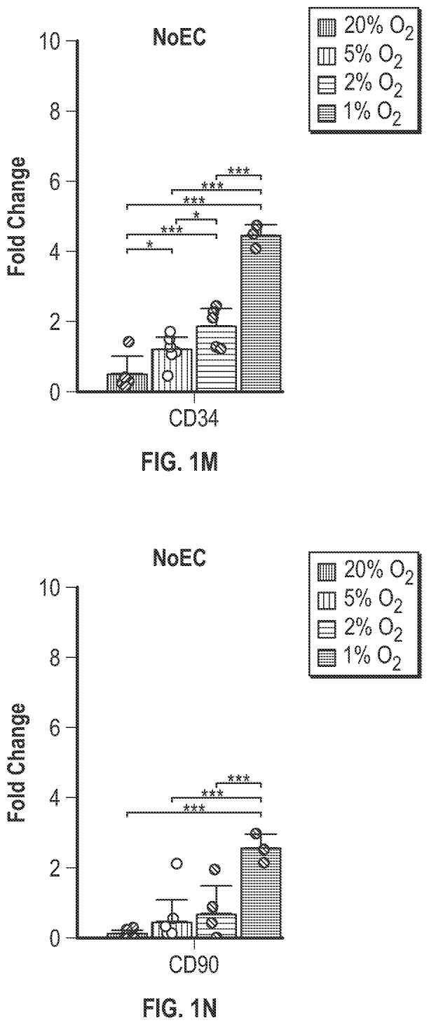

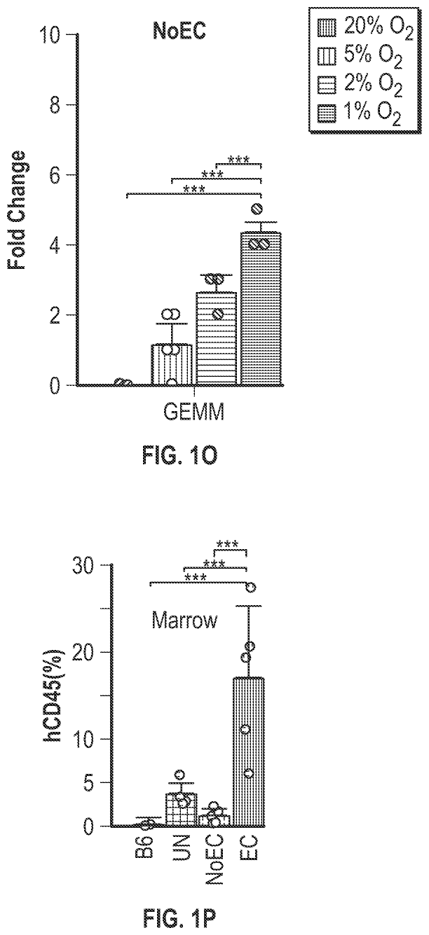

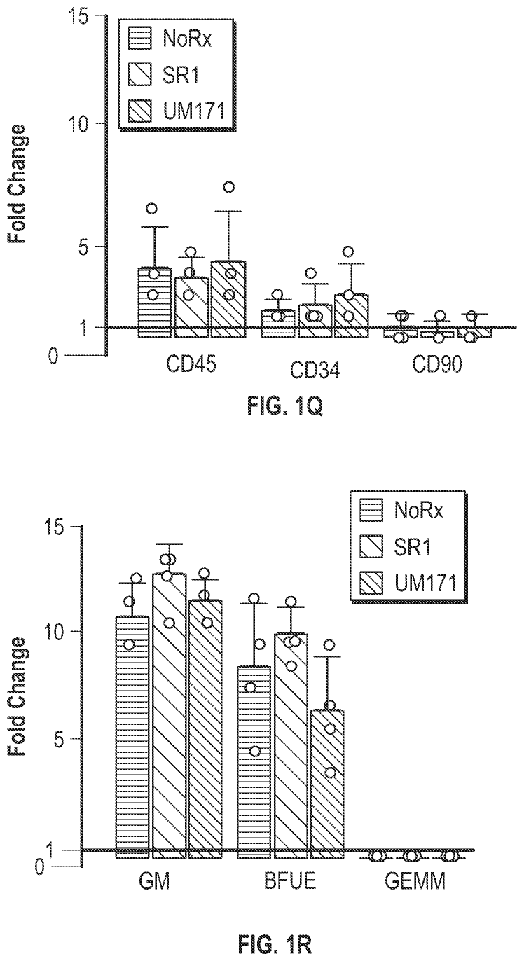

Initial experiments were performed to evaluate the role of endothelial cells on AD-HSPC growth. Mobilized AD-HSPCs were cultured for 7 days in serum-free media with and without endothelial cells (FIG. 1A). However, ex vivo culture of AD-HSPCs using endothelial cell (EC) feeder cells (mimicking the vascular niche platform) and conditions previously successful for CB-HSPCs failed to expand immature AD-HSPCs including immunophenotypically-defined HSCs (iHSCs) and CFU-GEMM (FIG. 1B-1C). Adult-Derived-HSPCs capable of long-term engraftment in vivo within immune-deficient NOD.Cg-Prkdc^{scid}Il2rg^{tm1Wjl}/SzJ (NSG) mice were lost during ex vivo propagation (FIG. 1E). Thus, new approaches were required to support ex vivo HSC self-renewal (FIG. 1D).

HSCs reside in hypoxic marrow regions where the oxygen sensor, hypoxia inducible factor 1-α (HIF1α), helps HSCs maintain quiescence and avoid stress. The inventors designed experiments to evaluate whether failure of the vascular niche platform to expand AD-HSPCs was caused by the non-physiologic oxygen tension used during ex vivo culture.

To test this, the Adult-Derived-HSPCs were cultured under different oxygen levels with endothelial feeder cells (FIG. 1F-1J) or without endothelial cells (FIG. 1K-1O) and the fold-expansion of immunophenotypic and functional HSPC populations was analyzed.

As illustrated in FIG. 1F-1O, lowering oxygen tension did not alter overall cell yield but prevented loss of immature immunophenotypes and functions. CD34 expression was reduced during culture in ambient oxygen (20% O₂) regardless of the presence of ECs (FIGS. 1H and 1M.

In contrast to the lost expression of CD38 commonly observed with ex vivo expansion of HSPCs (von Laer et al. Leukemia 14, 947-948 (2000); Ngom et al. Mol Ther Methods Clin Dev 10, 156-164 (2018)), the pattern of CD38 expression was maintained during culture in the presence of ECs suggesting that the vascular niche platform supported more physiologic behavior.

However, under hypoxia, ECs supported greater than 30-fold expansion of immunophenotypically-defined HSCs (iHSCs) and CFU-GEMM in contrast to just 1.5 to 4-fold expansion with hypoxia alone, without endothelial cells.

These immature iHSCs and CFU-GEMM populations collapsed when AD-HSPCs were cultured in ambient oxygen (FIG. 1H-1O; see especially 1I-1J, 1O). Engraftment into immunodeficient (NSG) mice of AD-HSPCs expanded in hypoxia with ECs was higher than that of unexpanded controls (FIG. 1P). Engraftment of AD-HSPCs cultured in hypoxia (1% oxygen atmosphere) alone, without endothelial feeder cells, was significantly lower than controls. The engrafted human CD45+ cells were deficient in either lymphoid or myeloid potential, indicating that HSC function had been lost during propagation (FIG. 1P). The results are summarized in the following table.

| | B6 | Un | NoEC | EC |
|---|---|---|---|---|
| Fraction | 0/5 | 16/20 | 8/20 | 20/20 |
| Engrafted | | | | |

In another experiment, mobilized AD-HSPCs were cultured for 7 days in serum-free media without endothelial cells to evaluate whether supplementation of the culture media with KIT ligand (KITL, 50 ng/mL), FLT3 ligand (FLT3L, 50 ng/mL), thrombopoietin (THPO, 50 ng/mL) and either SR1 or UM171 was able to maintain the phenotype and expand the AD-HSPCs.

As shown in FIG. 1Q-1R, in the absence of endothelial cells, the cell numbers of the different cell types did not change significantly compared to the untreated controls (NoRx). In addition, SR1 and UM171 failed to maintain the immunophenotype of HSCs (CD34+CD38-CD45RA-CD90+, iHSC) and those of the functional progenitors capable of mixed myeloid differentiation (i.e., colony forming units that generate myeloid cells (CFU-GEMM), abbreviated GEMM) (FIG. 1Q-1R).

Taken together, these studies show that differentiation and loss of immature adult-derived HSPCs were prevented by hypoxia but HSC maintenance and self-renewal required cues from endothelial cells and from hypoxia.

### Example 3: Robust Hematopoietic Reconstitution of NSG Mice Engrafted by Expanded AD-HSPCs

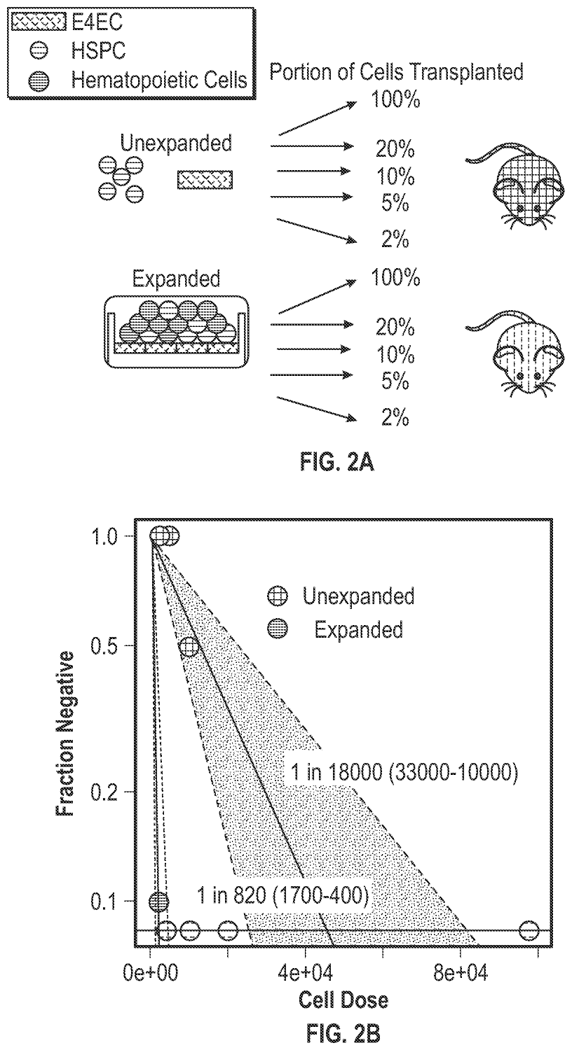

Limiting dilution transplantation is the standard (and best) assay for quantifying human HSCs. To quantify expansion of AD-HSPCs capable of long-term hematopoietic reconstitution within NSG mice, limiting dilution transplantation of AD-HSPCs was performed before expansion and of their expanded progeny as illustrated in FIG. 2A.

Calculated NSG-mouse repopulating cell (NRU) frequencies for unexpanded cells were similar to reported measurements of 1 in 18000 cells (95% CI, 10,000-33,000 (FIG. 2B) (Lidonnici et al. Haematologica 102, e120-e124 (2017)). In contrast, NRU frequency in hypoxic-expanded co-cultures was 1 in 820 (95% CI, 400-1,700; FIG. 2B). A 22-fold (95% CI, 6-83-fold) expansion of these primitive cells was realized upon use of the methods described herein (FIG. 2B). The table below shows the number of transplanted mice meeting engraftment criteria.

| Portion of Cells | | |
|---|---|---|
| Transplants | Unexpanded | Expanded |
| 100% | 3/3 | 3/3 |
| 20% | 5/5 | 3/3 |
| 10% | 5/10 | 5/5 |
| 5% | 0/10 | 9/10 |
| 2% | 0/10 | 9/10 |

These expansion results indicate significant symmetric self-renewal occurred during culture using the hypoxic vascular niche platform. Self-renewal means that both the progenies of a cell division have the phenotype of the parent HSC. In other words, both progeny retain their original HSPC phenotype. To evaluate whether ex vivo expansion skewed the lineage potential of AD-HSPCs, mice were transplanted with unexpanded controls or the same cells expanded using the hypoxic vascular niche platform. No bias was observed in the engraftment of lymphoid and myeloid cells in mice transplanted with unexpanded controls or the same cells expanded using the hypoxic vascular niche platform.

To test if expansion limited complete hematopoietic reconstitution in engrafted mice, clinically validated EuroFlow panels (Kalina et al., Leukemia 26, 1986-2010 (2012)) were used to extensively characterize the immunophenotype of engrafted mice. No aberrant immunophenotypes were observed and recipient mice were replete with cells marked by granulocytic, monocytic, erythroid, T cell. B cell and NK cells markers. The B and T cells were polytypic with both κ/λ light chains (B cells) and CD4/CD8 (T cells) easily identifiable. Thus, the hypoxic vascular niche platform was able to expand AD-HSPCs with all the features of bonafide HSCs.

### Example 4: Reducing Oxidative Stress and Stabilizing HIF1α Favors HSPC Expansion

Reactive oxygen species (ROS) are thought to reduce HSC self-renewal and promote their differentiation, potentially explaining why immature HSPC immunophenotypes and functions are lost during ex vivo propagation of AD-HSPCs in ambient oxygen. ROS can also damage HSCs causing them to die or develop mutations.

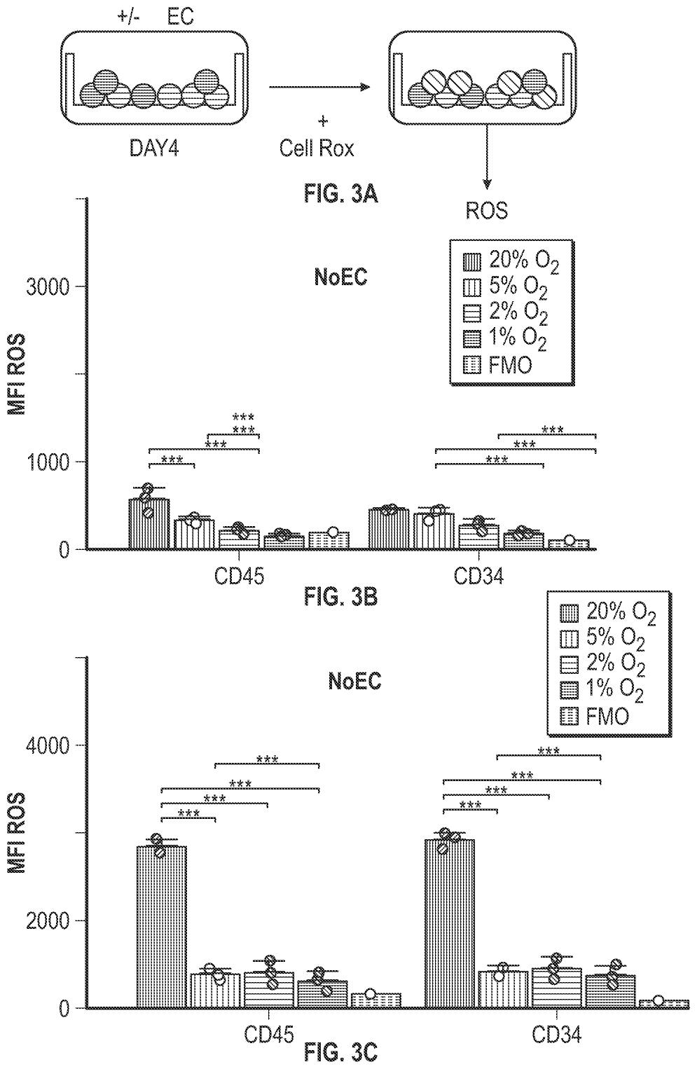

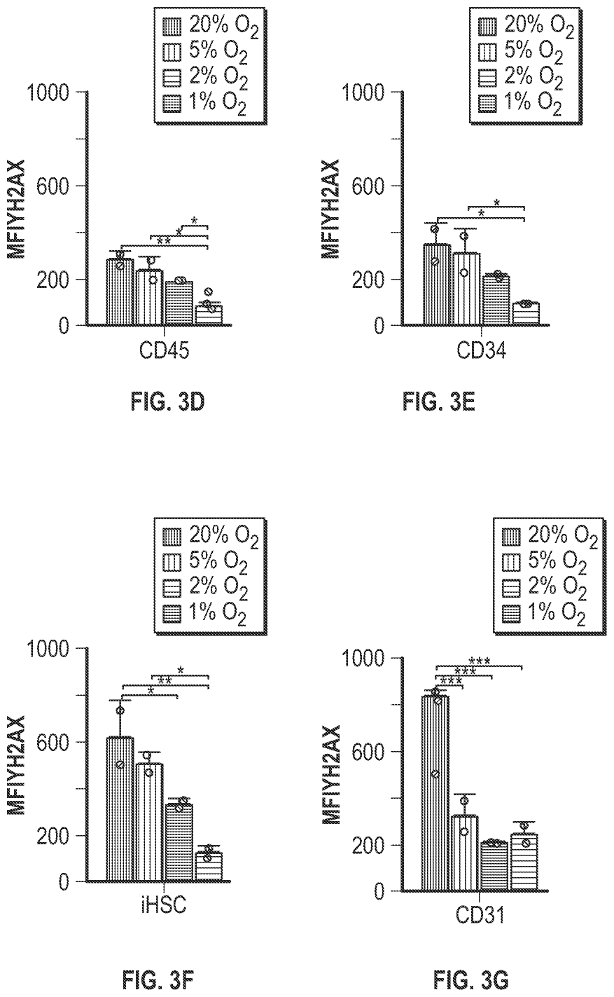

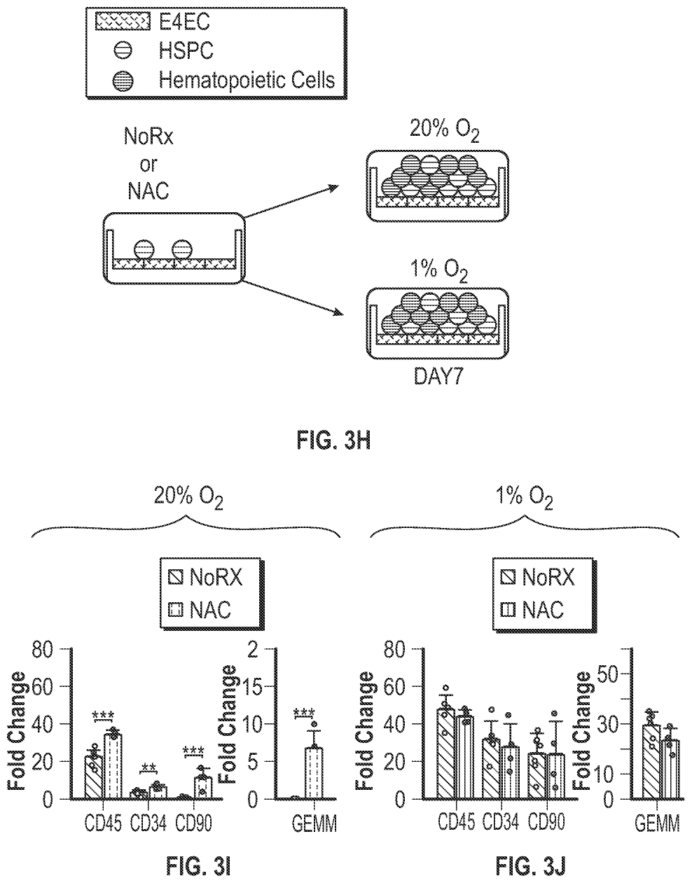

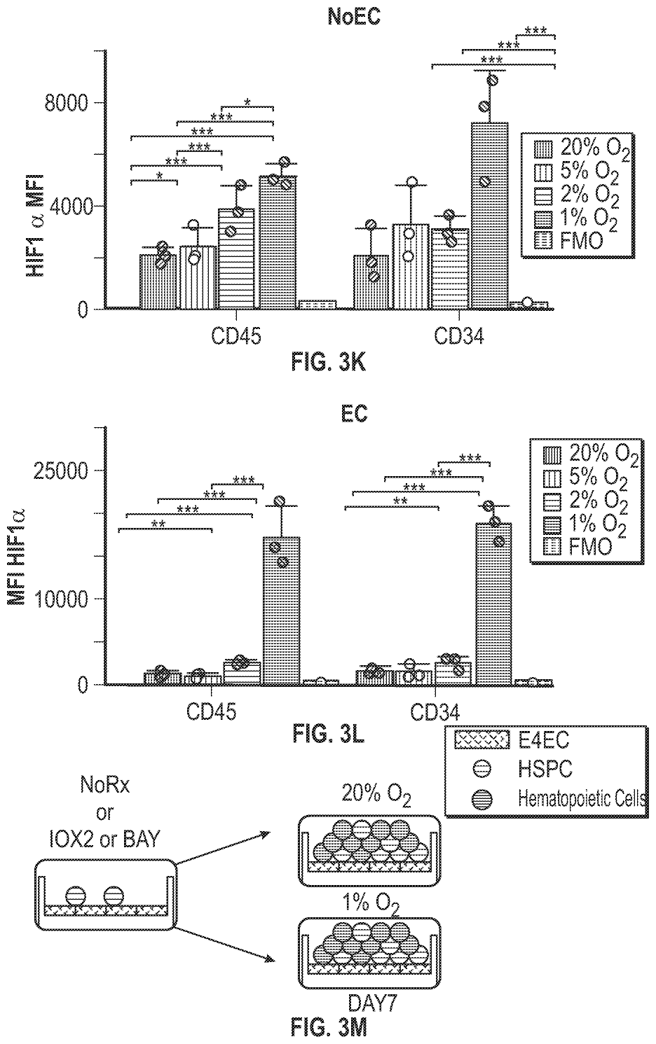

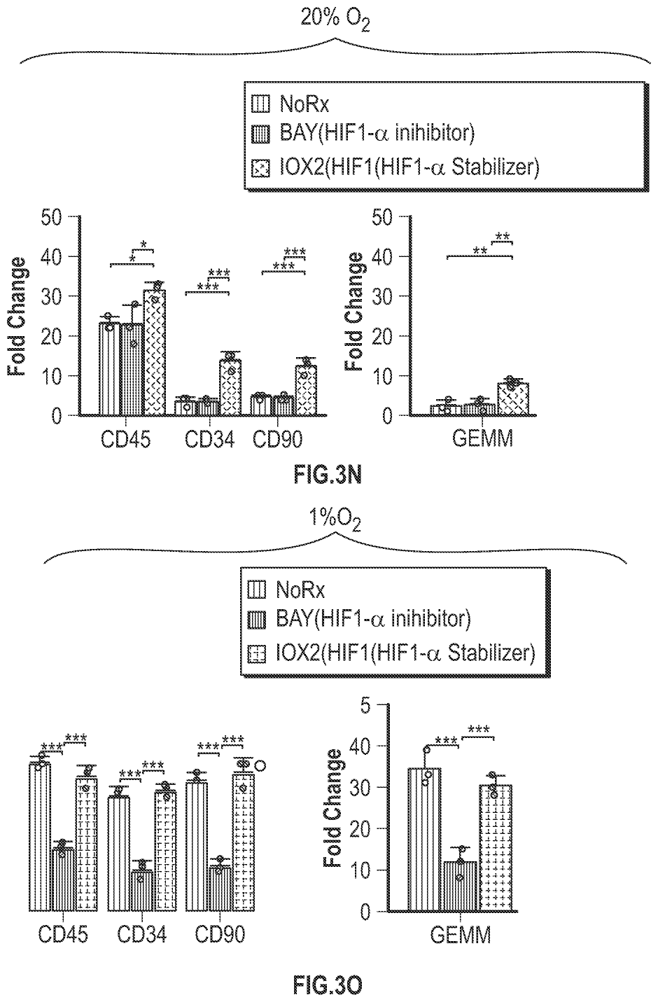

A fluorogenic CellRox probe was used to report ROS/oxidative stress in AD-HSPCs after propagation at various oxygen tensions (FIG. 3A). As illustrated in FIG. 3B-3C, both in the presence (EC) and absence of EC (NoEC), lowering oxygen from ambient (20%) to 5%, dramatically reduced ROS, as reported by CellRox mean fluorescence intensity (MFI). ROS was marginally reduced to an apparently basal level upon lowering oxygen to 2%. ROS was much higher at 20% oxygen in the presence of ECs, which may reflect the extensive proliferation and differentiation that occurs in that condition (FIG. 1F). Culture of AD-HSPCs in ambient oxygen is therefore associated with potentially damaging levels of ROS.

ROS are known to cause lipid peroxidation and consequent cell and DNA damage. Measurements indicated that lipid peroxidation was reduced 4-fold or more when the oxygen levels were lowered from 20% to 1% during AD-HSPCs propagation. Intracellular staining for γH2AX was used to report DNA damage. As shown in FIG. 3D-3J, hypoxia also reduced DNA damage in the AD-HSPCs.

To evaluate whether the ill-effects of ROS could be mitigated, N-acetyl cysteine (NAC), an antioxidant, was added to the cell cultures. As shown in FIG. 3H-3J, NAC broadly improved ex vivo propagation of even the most immature immunophenotypic and functional AD-HSPCs compared to the controls (NoRx). In contrast, NAC had no discernable effect when AD-HSPCs were propagated at 1% oxygen, presumably because ROS was already low in hypoxia (FIG. 3J). A similar protective effect was found when ascorbic acid (vitamin c) was used to quench ROS. These results show that standard culture at ambient oxygen (20%) induces significant oxidative stress in AD-HSPCs that likely limits HSC survival ex vivo.

In ambient oxygen, HIF1α is rapidly degraded, which potentially can phenocopy the detrimental effects of genetic HIF1α deletion on HSCs (Simsek et al. Cell stem cell 7, 380-390 (2010) Takubo et al. Cell stem cell 7, 391-402 (2010)). HIF1α levels in AD-HSPCs were directly measured after propagation at various oxygen tensions (FIG. 3K-3L). HIF1α protein increased significantly when oxygen was reduced below 5% and was highest at 1% O₂, as detected by HiF1α mean fluorescence intensity (MFI) in permeabilized cells (FIG. 3K-3L). HiF1α stabilization occurred both in the presence (EC) and absence of EC (NoEC), but was highest following culture using the vascular niche platform, indicating metabolic interactions may occur between AD-HSPCs and ECs. Thus, HIF1α signaling can vary significantly when oxygen tension is modulated during ex vivo propagation of AD-HSPCs.

Decoupling HiF1α stabilization from hypoxia by genetic deletion of the Von Hippel-Lindau gene (VHL) in vivo or use of proline hydroxylase domain (PHD) inhibitors to stabilize HIF1α in vitro can drive HSC quiescence, leading to reduced HSC numbers and repopulating activities. To assess the functional consequences of modulating HIF1α levels, a selective PHD inhibitor (IOX2) was used to stabilize HIF1α protein or BAY 87-2243 (BAY) was used to block HIF1α accumulation (FIG. 3M). As shown in FIG. 3N, HIF1α stabilization with IOX2 improved ex vivo propagation of CD45, CD34-expressing HSPCs, and CD90-expressing iHSCs as well as CFU-GEMM, compared to controls cultured when cultured in 20% oxygen without additional agents (NoRx). Prevention of HIF1α stabilization using BAY had no effect at 20% oxygen. In contrast, BAY significantly reduced expansion of AD-HSPCs when cultured on the (EC) vascular niche at 1% oxygen thereby indicating a functional role for HIF1α signaling in this culture system (FIG. 3N). Further stabilization of HIF1α with IOX2 yielded no additional expansion benefits when the cells were cultured at 1% oxygen (FIG. 3N), presumably because signaling was near maximal.

Thus, HIF1α signaling makes important contributions to the success of ex vivo AD-HSPC propagation using the redox-optimized vascular niche platform. In animal models, HIF1α signaling increases HSC quiescence and reduces differentiation. As shown herein, reduced differentiation and proliferation occurred when AD-HSPCs were cultured in the absence of endothelial cells (ECs). However, rather than hypoxia or HIF1α stabilization promoting quiescence, HSCs appeared to undergo significant self-renewal in cultures using the endothelial cell vascular niche platform. These results indicate that hypoxia alters the niche as well as the HSPCs.

### Example 5: Oxygen Tension Modulates Angiocrine Vascular Niche Function

The activation state of AKT and MAPK signaling modulates the repertoire of angiocrine factors expressed by endothelial cells (ECs), and their capacity to promote murine HSC self-renewal in vivo and ex vivo.

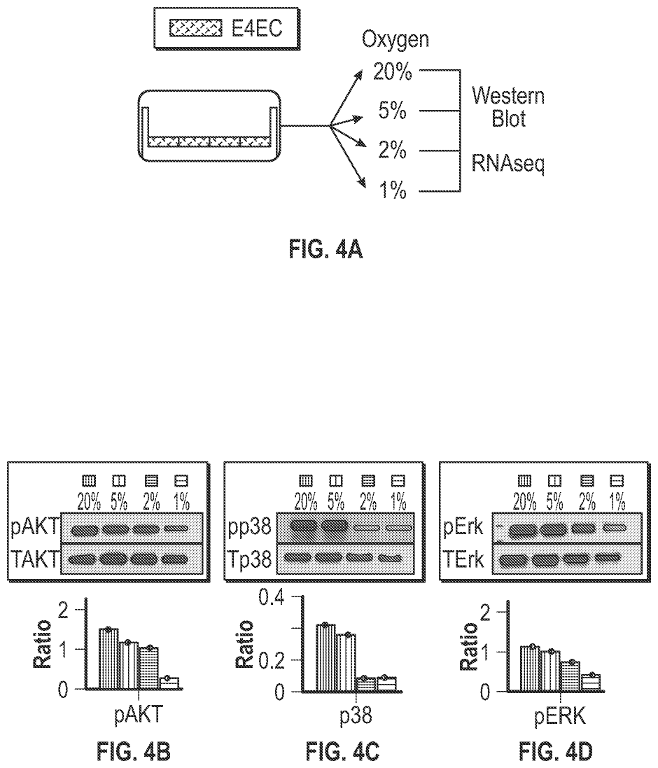

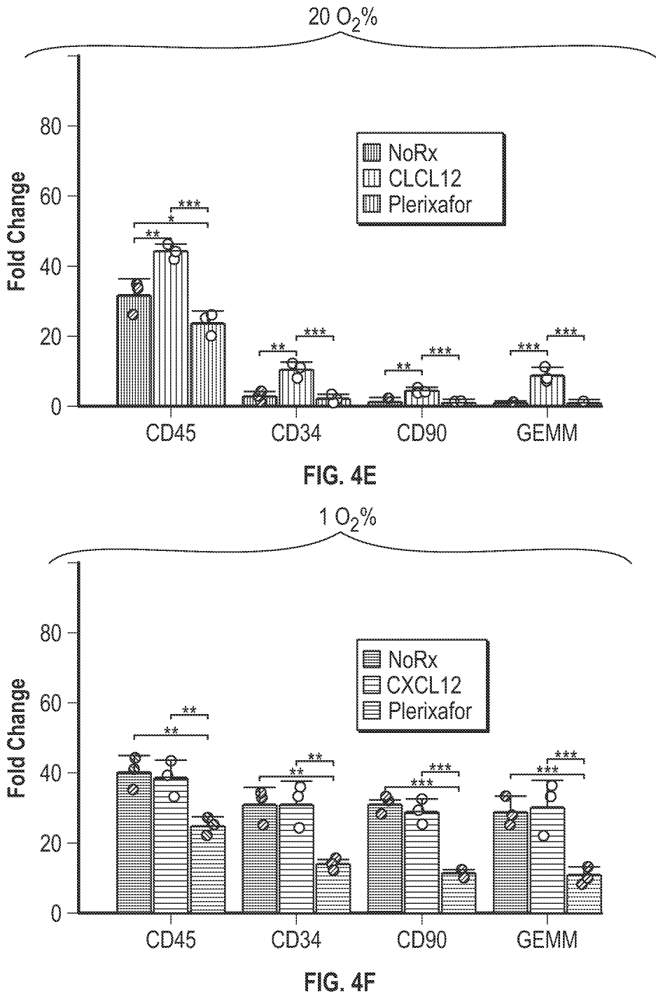

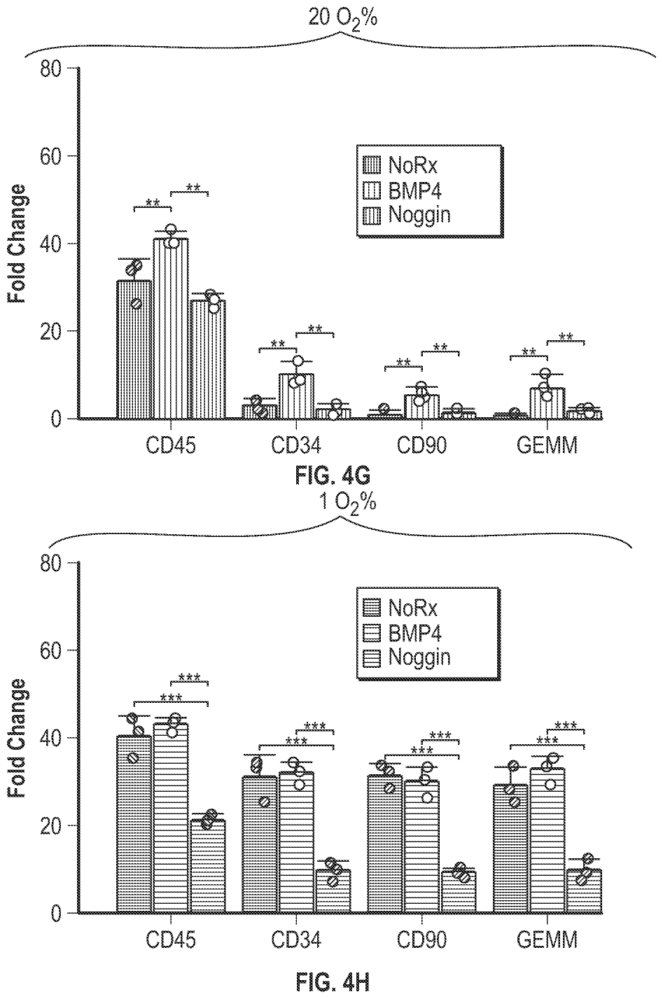

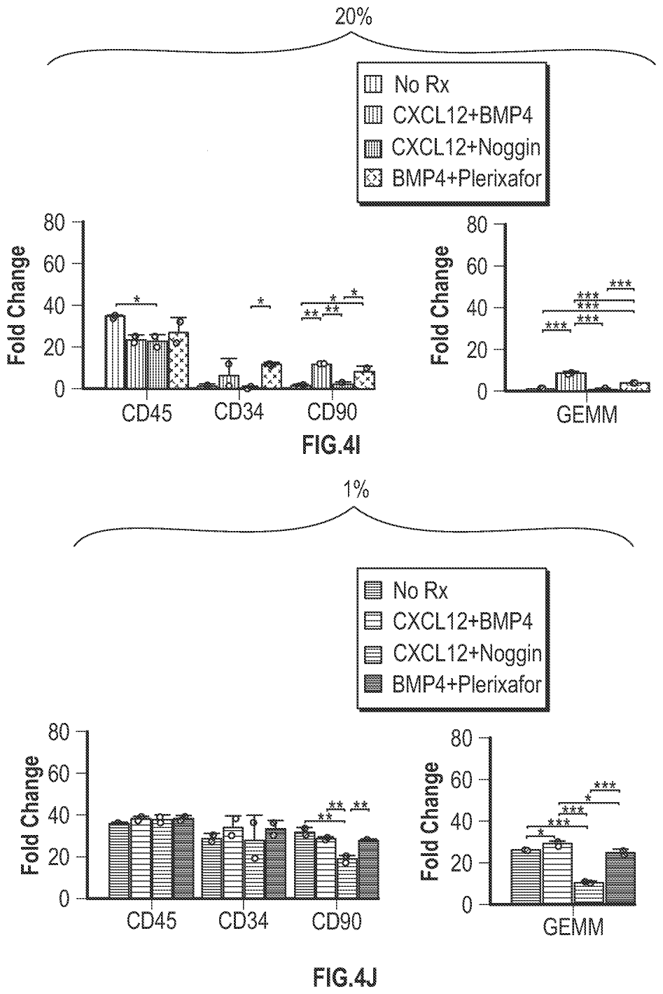

As shown in FIG. 4A, lowering oxygen tension reduced signaling through these pathways in the vascular niche platform described herein, as detected by phosphorylation of AKT1 (AKT), MAPK14 (p38) and MAPK1 (ERK) (FIG. 4B-4D). RNAseq was performed and linear models were used to identify differentially regulated genes for each change in oxygen. Over 1000 genes were regulated by oxygen in endothelial cells, including many factors with known or likely angiocrine functions. Because the transcriptional patterns at 1% and 2% oxygen were virtually indistinguishable—only three gene clusters were differentially regulated, and one was a pseudogene. These three gene clusters were grouped into a “hypoxia” category. Four major clusters of regulated genes were identified with the largest groups comprised of genes that continuously increased (Cluster 1) or decreased (Cluster 4) expression in response to reduced oxygen tension. Cluster 2 identified genes with expression exclusively activated in hypoxic conditions (1% or 2% oxygen) whereas Cluster 3 was comprised of a small number of genes exclusively upregulated at 5% oxygen. Gene sets related to hypoxia and glycolysis were enriched by hypoxia whereas MTORC1, oxidative phosphorylation and fatty acid metabolism were among the most highly ranked sets downregulated. DNA repair genes were both strongly downregulated by hypoxia in agreement with flow cytometry for lipid peroxidation and DNA damage. The angiogenic functions of ECs are promoted by hypoxia. However, the results described herein demonstrate that changes in oxygen tension also reshape angiocrine function.

Endothelial cells deploy angiocrine factors of many families and functions. Changing oxygen tension was found to induce an angiocrine switch with expression of new cohorts of chemokines and cytokines coincident with silencing of others. Whereas the inflammatory chemokines CXCL8 (IL8), CCL2 (MCP1) and CCL20 (MIP3A) were strongly downregulated (between 6.7 and 3.9-fold), CXCL12 (SDF1) was upregulated almost 230-fold in hypoxia from barely detectable expression at 20% oxygen.

HSCs express the CXCL12 receptor. CXCR4. The inventors tested the dependence of HSC expansion on this angiocrine signal by supplementing cultures with CXCL12 or blocking its function with the CXCR4 inhibitor, plerixafor (FIG. 4E-4F). In ambient oxygen (20%), exogenous CXCL12 increased iHSC and CFU-GEMM expansion compared to controls cultured without additional agents (NoRx) but CXCL12 had no detectable effect in hypoxia (FIG. 4E-4F). In contrast, blockade of CXCL12 signaling using plerixafor significantly limited ex vivo expansion only in hypoxia, indicating that this pathway is exclusively deployed by the hypoxic vascular niche (FIG. 4E-4F).

The RNAseq analysis described above showed that genes within the TGFβ superfamily were strongly regulated by hypoxia. Among the most prominent upregulated genes was BMP4, a gene implicated in HSC self-renewal (Goldman et al. Blood 114, 4393-4401 (2009)). To assess the contribution of BMP4, aHSPCs were cultured using our vascular niche platform with added BMP4, the BMP inhibitor, NOG (Noggin), or vehicle (NoRx). As shown in FIG. 4G-4H, BMP4 increased expansion of immature aHSPCs cultured at 20% but not at 1% oxygen. Inhibition of BMP4 using Noggin had the converse effect: reducing expansion of aHSPCs in hypoxia (1% oxygen) but demonstrating no activity at ambient oxygen (20%) (FIG. 4G-4H). These results indicated the angiocrine switch triggered by hypoxia is at least partly driven by BMP signaling as well as CXCL12 signaling.

The similar effects of CXCL12 and BMP4 suggested a linkage between these pathways during vascular niche expansion of aHSPCs. To identify interactions, aHSPCs were cultured using the vascular niche and the following combinations of additives: BMP4 with CXCL12; CXCL12 with Noggin; BMP4 with plerixafor; or no addition (NoRx). Adding both CXCL12 and BMP4 during propagation yielded no greater expansion than either agent alone, suggesting that these pathways may be signaling through the same downstream effector proteins (FIG. 4J). Expansion of aHSPCs in the vascular niche platform with both plerixafor and BMP4 was not distinguishable from addition of BMP4 indicating that CXCR4 signaling is dispensable when BMP4 signaling is intact (FIG. 4J). In contrast, adding Noggin mitigated the aHSC expansion activity of CXCL12 (FIG. 4J) indicating that a Noggin client such as BMP4 is downstream of CXCL12 signaling.

Taken together, these data indicate that hypoxia reprograms the vascular niche platform by reducing aHSPC stress and inducing an angiocrine switch to factors more favorable for HSC self-renewal.

### Example 6: Symmetric HSC Self-Renewal During Vascular Niche Propagation

Symmetric self-renewal is necessary for expansion of HSCs. Limiting dilution analysis indicates that the HSC frequency increases during propagation using the vascular niche platform (FIG. 2B).

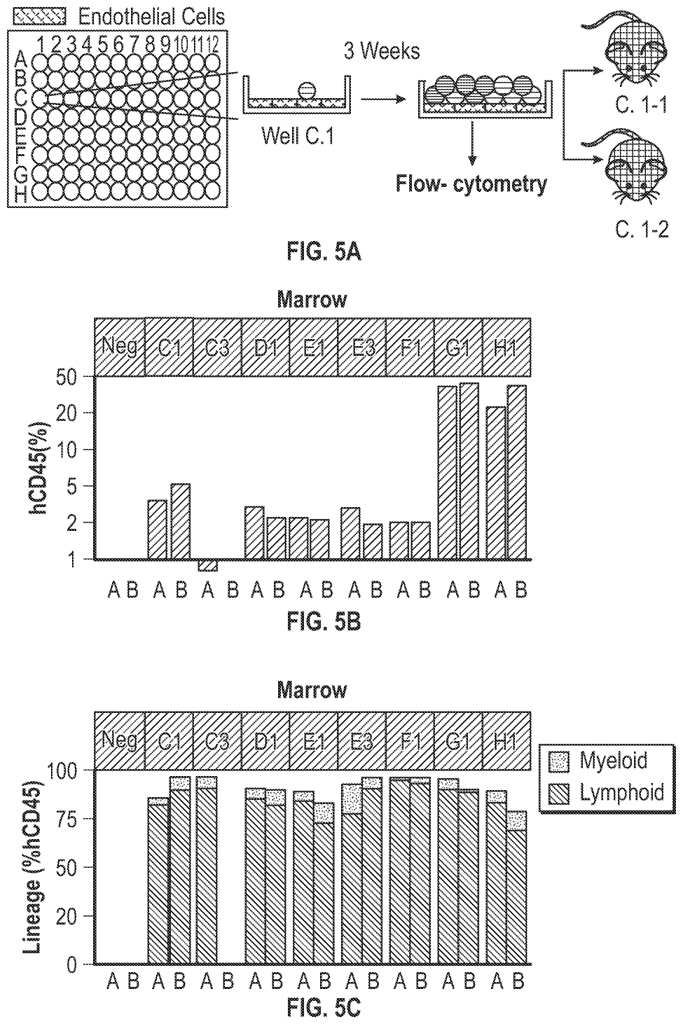

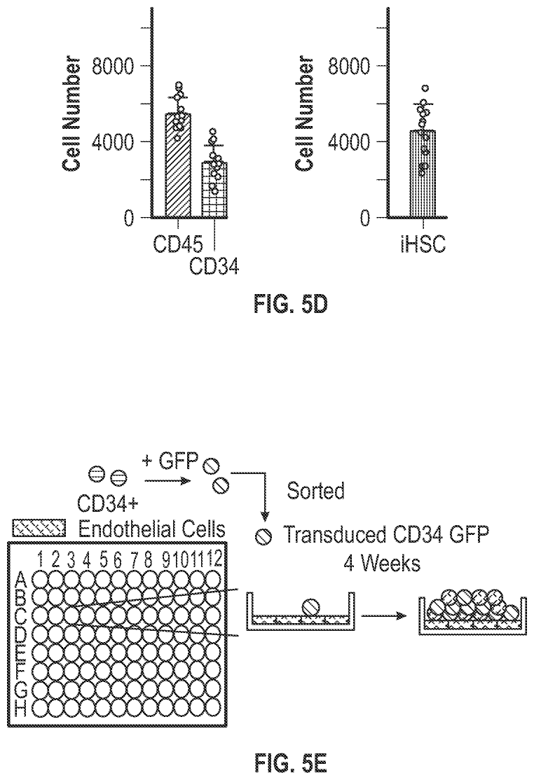

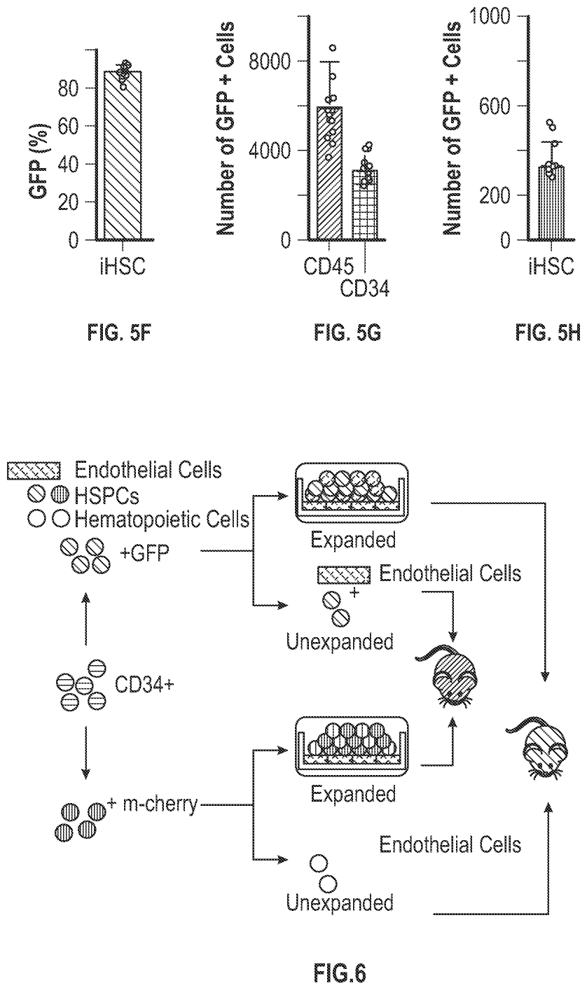

To eliminate competing explanations for increased HSC frequency-such as alterations in HSC homing or engraftment-individual iHSCs were expanded using the hypoxic vascular niche platform. Single HSCs were propagated in this way for 3 weeks in a 96-well plate format prior to analysis (FIG. 5A). Cell numbers were quantified by immunophenotype. NSG repopulating cells are capable of engrafting NSG mice with all human hematopoietic lineages thus defining hematopoietic stem cell activity. To assess NSG-repopulating cell (NRC) numbers, the progeny of a single iHSC from eight randomly selected wells were split into two portions and each portion was transplanted into a different NSG mice. Mice were analyzed 20 weeks after transplantation and hCD45-derived myeloid and lymphoid engraftment was assessed by flow cytometry in peripheral blood and bone marrow.

As illustrated in FIG. 5B-5C, 15 of the 16 transplanted mice exhibited detectable multilineage human hematopoietic engraftment. The one exception was a mouse that died prior to analysis. The progeny from all eight iHSCs were able to engraft at least one NSG mouse and the progeny of 7 of 8 iHSCs engrafted both NSG mice by 20 weeks.

The expansion of iHSC progeny from the single cells incubated in different wells of the 96-well plates was quantified after 21 days propagation (FIG. 5D). Each iHSC yielded about 5500 CD45+ cells and about 460 iHSCs, indicating that approximately 12 doublings had occurred and approximately 8.8 self-renewal divisions of iHSCs occurred during the 21 days of culture.

Human HSCs are thought to divide approximately every 40 (25-50) weeks in vivo (Catlin et al., Blood 117, 4460-4466 (2011)). Thus, the three-week ex vivo expansion is roughly equivalent to 6.8 (4.2-8.5) years of in vivo cell division. These data provide definitive proof of symmetric self-renewal during ex vivo propagation using the hypoxic vascular niche platform. The methods described herein can therefore provide sufficient quantities of human HSCs to be therapeutically useful for treatment of a variety of diseases and conditions.

### Example 7: Ex Vivo Expansion of Genetically Modified Adult-Derived HSCs Using Redox-Optimized Vascular Niche Platform

Autologous gene therapy requires production and/or maintenance of genetically modified HSCs in quantities sufficient for safe hematopoietic stem cell transplantation (HSCT). To assess expansion of engineered HSCs, mobilized peripheral blood (mPB) CD34+ were transduced with a lentivirus expressing GFP and single iHSCs were then sorted individually for vascular niche expansion (FIG. 5E). Transduced iHSCs were co-cultured for 3 weeks and quantified by flow cytometry. High percentages of iHSCs exhibited sustained GFP expression (FIG. 5F), indicating that transgene expansion was not silenced during propagation. Each single transduced iHSC yielded an average of 5800 CD45+GFP+, 2800 CD34+GFP+ and 330 GFP+iHSCs (FIG. 5G-5H). Taken together, these data demonstrate that the redox-optimized vascular niche platform can fill a critical gap in gene therapy by enabling expansion of modified HSCs that provide stable transgene expression.

### Example 8: Competitive Cell Repopulating Assay

A competitive repopulating assay was developed to compare the engraftment of transduced aHSPCs that were either unexpanded or expanded using the vascular niche platform (FIG. 6). Mobilized peripheral blood CD34+ HSPCs were transduced with lentivirus expressing GFP or lentivirus expressing mCherry. After transduction, half of the transduced cells were cryopreserved right away. The other half of the transduced cells was expanded for a week using the hypoxic vascular niche platform and then cryopreserved.

Cells were later thawed. Thawed GFP cells were mixed with thawed mCherry cells, so that two mixtures of labeled cells were generated. Each mixture contained labeled expanded and labeled unexpanded cells, where the expanded and unexpended cell were differently labeled (see FIG. 6). The different cell mixtures were separately transplanted into NSG recipient mice as shown in FIG. 6. Recipient mice were analyzed 20 weeks after transplantation and the proportion of GFP+ or mCherry+ cells were used to assess relative engraftment of expanded and unexpanded aHSPCs.

GFP and mCherry expression was high in unexpanded and expanded iHSCs. Results obtained by flow cytometry indicated that more than 74% of hCD45+ cells were marked by fluorescent protein. Significantly, engraftment of expanded cells was 4.4 to 5.2-fold higher than unexpanded cells within each co-transplanted mouse. These results demonstrate robust expansion of genetically marked cells, and that such expanded genetically marked cells are capable of long-term engraftment.

### Example 9: Vascular Niche Platform Expansion of Mobilized Human Sickle Cell Disease Peripheral Blood HSPCs Engineered to Express Non-Sickling β-Globin

Autologous gene therapy for sickle cell disease (SCD) offers great potential but patients frequently can provide only low numbers of cells for gene therapy and without adequate procedures for expansion of those low number of cells, there are inadequate amounts of cells for engraftment.

Plerixafor was used to mobilize HSPCs from the peripheral blood of two different donors with sickle cell disease. CD34+ cells were enriched from the cells collected using CliniMacs immunomagnetic beads and clinical, GMP processing. The total cells obtained at this stage were inadequate for sickle cell disease gene therapy. In general, auto-transplantation into a patient is best performed with 10 million cells per kilogram using mobilized donor sources. However, 5 million/kg can be used for gene therapy when there is no other option. Currently, transplantation with less than 5 million/kg often results in high risks of graft failure. Transplantation with less than 2 million/kg mobilized HSPCs is considered unsafe due to risk of graft failure. Using the methods described herein, 0.64 to 2.4 million CD34+/kg can be collected because the collected cells can readily be expanded, even after genetic repair or genetic modification of the cells.

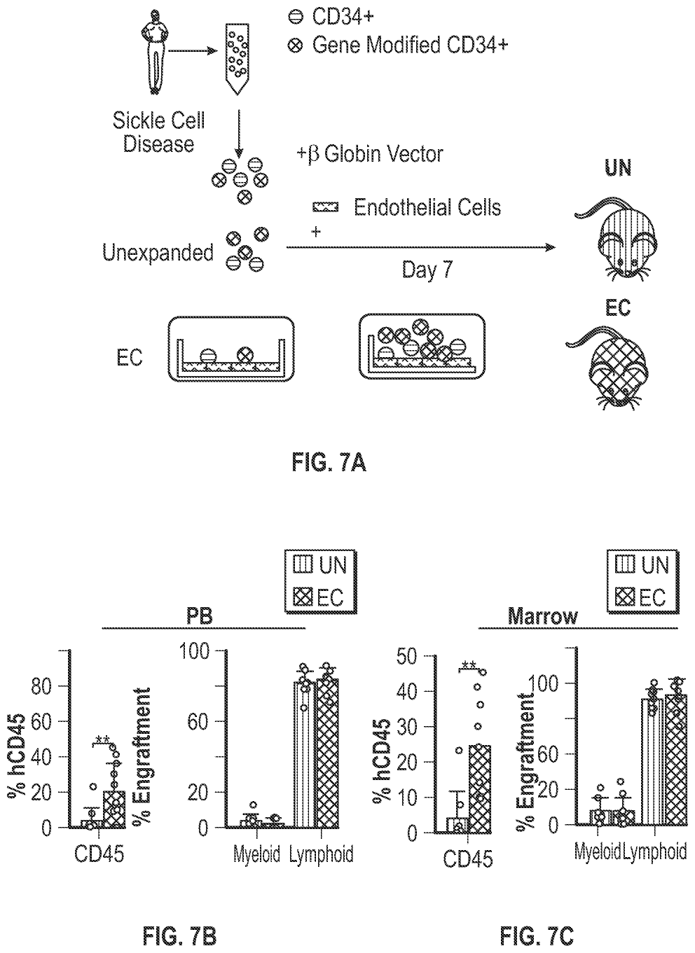

Sickle cell disease aHSPCs were transduced using a clinical-grade lentiviral vector (LV) expressing a non-sickling β-globin gene under control of regulatory elements from the locus control region (LCR). Processing was as described above for the GFP/mCherry competitive repopulation experiment (Example 8), except that expanded or unexpanded cells were transplanted into NSG mice without mixing the expanded and unexpanded cells (FIG. 7A). Recipient mice were analyzed 20 weeks after transplantation.

Expanded β-globin-transduced aHSPCs from sickle cell disease patients were engrafted into NSG mice at significantly higher levels in both the peripheral blood (6.3% expanded vs 1% unexpanded) and in the marrow (14% expanded vs 1.5% unexpanded). Engrafted mice exhibited no bias regarding the proportions of human myeloid and lymphoid cells, independent of expansion state (FIG. 7B-7C). The vector copy number (VCN) remained high in engrafted cells and erythroid differentiation of engrafted CD34+ cells demonstrated expression of the non-sickling D-globin transgene.

In another series of experiments, bone marrow HSPCs was obtained from three children with Fanconi Anemia (FA). Note that FA cells are extremely fragile and there has never been a successful gene therapy for FA despite 20+ years of effort. Marrow was obtained from the children and the cells were expanded using the vascular niche methods described herein.

The HSC expansion platform was used to expand bone marrow samples from FA patients. Overall, three primary FA patient bone marrow samples were expanded ex vivo using the hypoxic, endothelial feeder cell methods described herein. Expansion was assessed using immunophenotypic and functional assays of HSPCs.

First, CD34+ cells were selected and approximately 4000/cm CD34+ cells from the donors were separately seeded onto endothelial feeder cells (strain Ad5FY9, seeded at about 40,000/cm). However, as others have observed, the inventors found that immunomagnetic (Miltenyi) CD34+ enrichment works poorly for FA HSPCs leading to poor CD34 enrichment purity and yield. The low yield impeded accurate quantification of expansion but was estimated to be about 20-fold to 40-fold expansion of CD34+ HSPCs.

In a second experiment, the conditions were changed for FA donor #3 to utilize an increased seeding density by expanding the total nucleated cells (TNC; about 70,000 TNCs including about 1200 CD34+ cells). Using this new approach, a higher numbers of cells were obtained for the FA #3 donor—about 585-fold expansion of the FA #3 CD34+ HSPCs after 7 days propagation on the hypoxic vascular niche.

The following table summarizes the fold expansion obtained for CD34+ cells from the three FA cell donors.

| Fanconi Anemia | Unexpanded CD34+ | Expanded CD34+ |
|---|---|---|
| Donor | Cell Numbers | Cell Numbers |
| FA Donor 1 | 18150 | 432000 |
| FA Donor 2 | 5460 | 100000 |
| FA Donor 3 | 5940 | 246200 |
| TNC-Donor3 | 1196 | 699840 |

The expanded FA cells from FA donor #3 from the second experiment were also capable of NSG engraftment within one month of administration (9% of CD45).

Such expansion and engraftment of autologous cells has never before been done with Fanconi Anemia (FA) cells. The major barriers to FA gene therapy are the low number of HSCs from patients with FA and the loss of HSCs that occurs during gene therapy processing. Expansion of FA HSPCs using the hypoxic vascular niche overcomes these obstacles.

Taken together, these data indicate that the novel vascular niche platform and methods described herein can fill a much-needed therapeutic gap to promote autologous gene therapy for mono-genic blood disorders when donor cells have previously been unavailable in adequate numbers or when the cells require special handling to prevent HSC damage due to oxidative stress.

### Example 9: Microcarriers Containing Endothelial Cells for HSPC Expansion

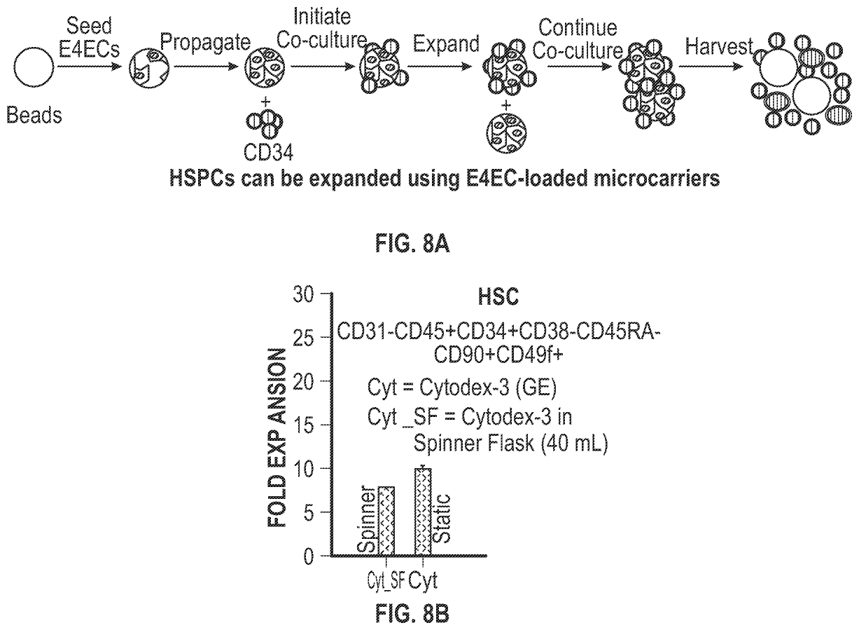

Endothelial cells that express E4ORF1 (E4ECs) were seeded into Cytodex-3® microcarriers and the microcarrier-ell combination was cultured to allow the endothelial cells to populate the microcarriers as illustrated in FIG. 8A. CD34+ cells were introduced to the cultured microcarriers. The mixed culture was incubated to allow the CD34+ cells to expand. In some cases, the additional microcarrier-endothelial cells were added after a few days of incubation to further promote expansion of the CD34+ cells. After expansion, the CD34+ cells are harvested. During harvesting, some of the endothelial cells can also be collected with the CD34+ cells. See FIG. 8A. However, the presence of endothelial cells, particularly natural (not genetically modified endothelial cells) is not a problem. For example, endothelial

FIG. 8B graphically illustrates fold expansion when using a microcarrier-based system, either with static cultures or in spinner flasks. Cytodex-3® microcarriers (Cyt; dextran beads coated with denatured porcine-skin collagen bound to their surface) were used in this experiment to expand HSPCs under low oxygen, vascular niche culture conditions. The microcarrier-based systems can be upscaled 40-fold without modification. The inventors have screened 14 microcarriers for suitability in vascular niche culture and have developed scalable microcarrier based expansion of AD-HSPCs using cGMP processes. In some cases, the microcarrier used was entirely dissolvable using specific enzymes such as collagenase, neutral proteinase, trypsin and pectinase. Dissolvable microcarriers can be entirely removed during harvest of expanded HSPCs using the vascular niche. The inventors have developed processes for removal of microcarriers after expansion. Such microcarrier-based expansion is efficient, inexpensive and useful for expansion of therapeutically effective numbers of cells, even if the cells are fragile and genetic modification is needed to repair a genetic defect in the cells.

### Example 10: Expansion without FA

One goal of the inventors was to find a way to expand human hematopoietic stem cells and/or hematopoietic progenitor cells without the need for E4ORF1 transfection of endothelial cells.

To achieve that goal, the inventors evaluated the expansion of adult HSPCs under different oxygen tensions in the presence of endothelial cells that did not express E4ORF1 while including different cytokines, with or without heparin in culture media that did not contain any serum.

Expansion of marrow-derived HSPCs was evaluated in the presence of various cytokines (at 10 ng/mL), including FGF2, EGF, IGF, heparin (HEP), and combinations thereof. Endothelial cells (about 40,000/cm) that did not express E4ORF1 were seeded onto plates containing NYSTEM medium that did not contain serum. As a control, the endothelial cells were also seeded into medium that had been treated with CTR adsorbent, which effectively adsorbs small- to middle-sized proteins such as cytokines and enterotoxins. The medium was replaced with StemSpan FTK media and HSPCs or CD34+ cells (5000/cm) were then seeded into the culture plates, followed by incubation at 370 in a chamber with 1% oxygen atmosphere.

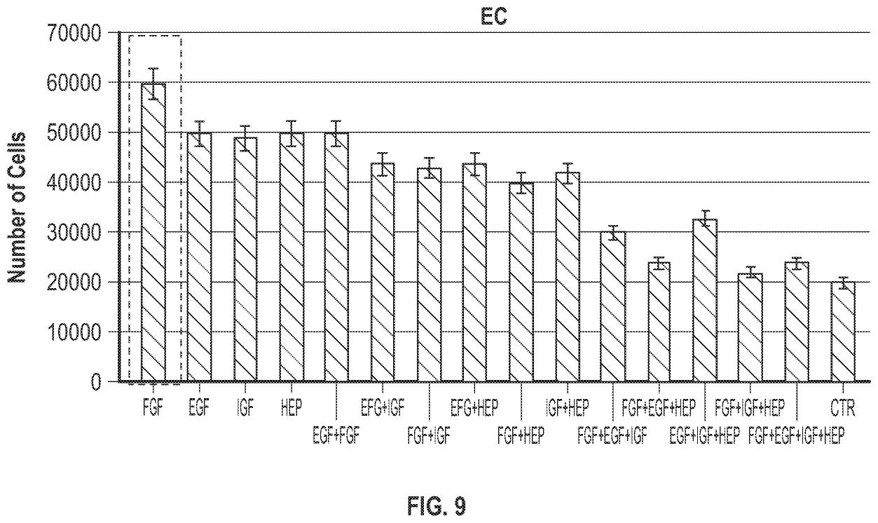

As shown in FIG. 9, all of the FGF2, EGF, IGF, HEP, or combinations thereof stimulated expansion but media containing only FGF2, without EGF, IGF, HEP, or combinations thereof effectively promoted the expansion of HSPCs even without E4ORF1 expression by endothelial feeder cells.

An E4-independent co-culture system was developed that expands HSPCs essentially as well as when using E4-expressing endothelial cells. This E4-independent co-culture system involved use of non-E4-expressing endothelial cells in co-culture with AD-HSPCs with FGF alone in StemSpan media containing THPO, KITL, and FLT3L. Approximately 24-40-fold expansion was achieved of the iHSCs. Accordingly, non-E4-expressing endothelial cells are useful for vascular niche expansion of hematopoietic stem and progenitor cells.

### Example 11: Screening for Agents that Deplete Myeloproliferative Neoplasm (MPN) Stem and Progenitor Cells (MPN-SPCs)

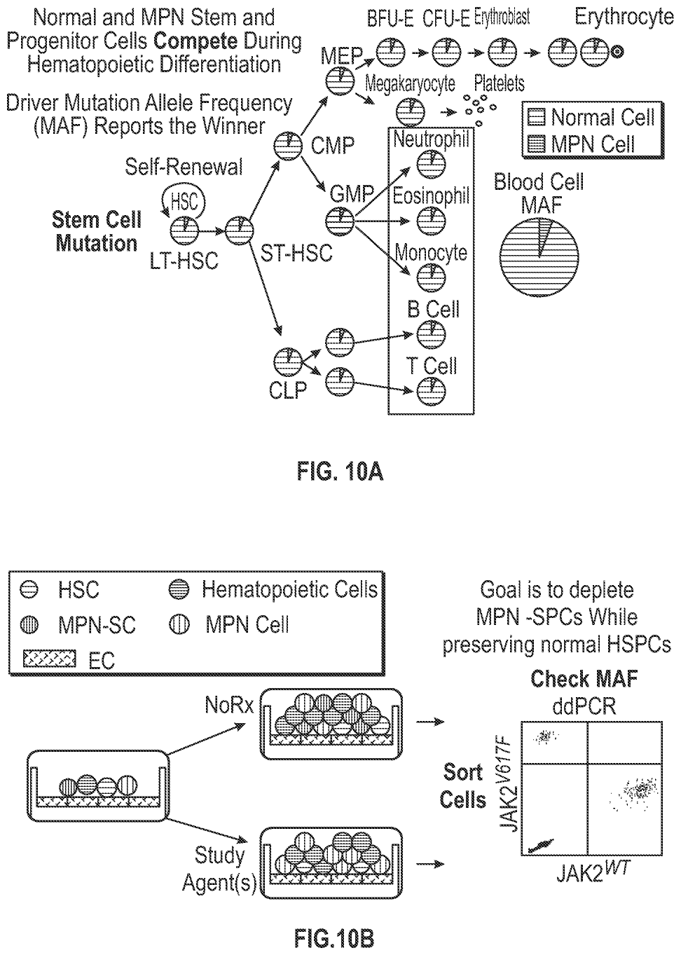

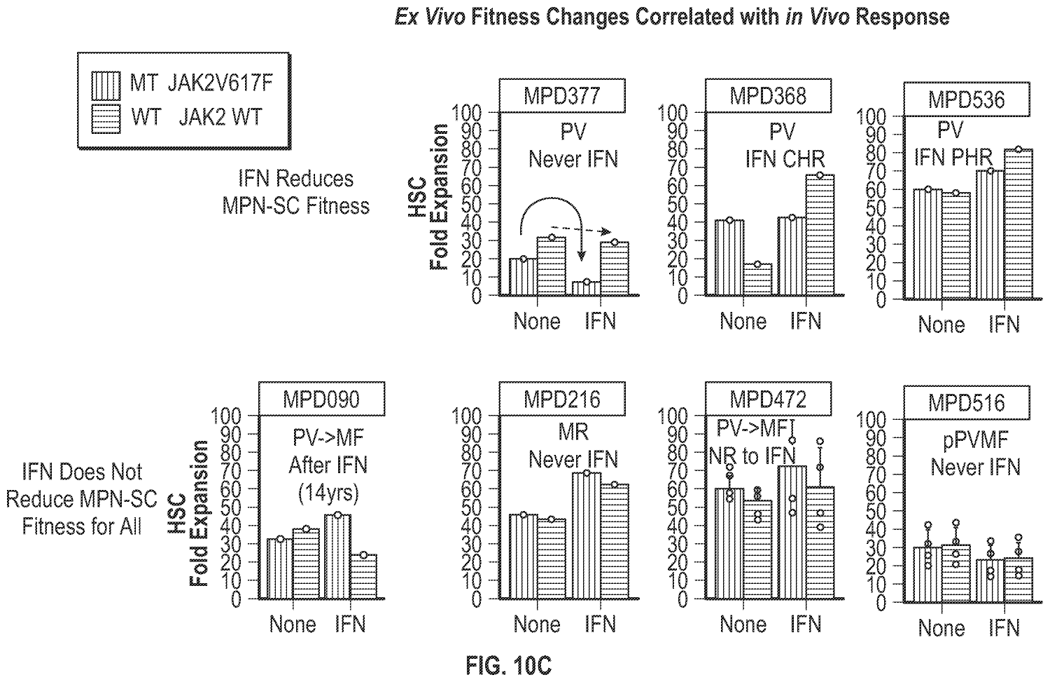

As illustrated in FIG. 10A, normal and neoplastic stem cells compete during hematopoietic cell differentiation and the proportion of normal and neoplastic cells that evolves during differentiation depends upon the mutation allele frequency (MAF) and fitness of the normal/mutant cells. For example, the JAK2 V617F mutation is an acquired, somatic mutation present in the majority of patients with myeloproliferative cancer (myeloproliferative neoplasms). A chronic malignancy can begin with one mutated hematopoietic stem cell (HSC). Hence, reduction or elimination of neoplastic stem and progenitor cells (e.g., JAK2 V617F mutant stem cells) from the population of hematopoietic stem cells can reduce or eliminate those neoplastic cells from the population of differentiated cells that arise from the stem and progenitor cells.

Animal models indicate that MPN stem cells (MPN-SC) are the genesis of MPN phenotypes and MPN disease—and that even a single MPN-SC can fully recapitulate the disease. For this reason, it is widely recognized that cure of MPNs requires therapy that can eradicate or cripple MPN-SCs. But this has proven difficult to do because MPN-SC share immunophenotypes and biological properties of normal HSCs. There is currently no suitable model system for testing whether new agents, or combinations of agents, are useful for selectively targeting MPN-SCs while sparing the normal HSCs that are necessary for life. For this reason, the inventors established an in vitro system with which MPN-SC targeting agents/combinations can be efficiently identified. This is a significant advance because cell line models do not adequately capture (mimic) the salient stem cell biology, murine models are expensive (as well as being slow and overly simplistic), and clinical trials are prohibitively slow and expensive require consideration of ethical issues that necessarily limit the speed and spectrum of study. Indeed, clinical trials do not include critical endpoints such as overall and progression-free survival because these endpoints take too long and are prohibitively expensive.

A screen was developed for identifying agents useful for identifying, depleting, and eliminating neoplastic cells from a population of hematopoietic stem cells collected from a patient or donor. The screen is useful, for example, when collecting stem cells from the bone marrow or peripheral blood cells of cancer patients. For example, simply reintroducing such stem cells back into the cancer patients after the patients have undergone radiation treatment or chemotherapy can reintroduce any neoplastic cells that are present in the collected cells. Use of healthy allogeneic hematopoietic stem cells is an option but the allogeneic cells can still cause rejection, bacterial infections, invasive fungal infections, and viral infections.

The screening methods described herein can identify agents that are uniquely capable of identifying and depleting the type of neoplastic cells that a patient may have, thereby allowing efficient and cost-effective identification of agents that can effectively treat hematological neoplasms. Treatment of the patient's hematopoietic stem cells, or selection of only healthy, non-cancerous cells for expansion, can provide the patient with much needed disease-free hematopoietic stem cells.

As illustrated in FIG. 10B, the screen involved collection of hematopoietic stem cell (HSC) from the blood or marrow of a patient or healthy donor, followed by incubation of a series of HSC aliquots with different test agents, and then detection of the number or proportion of neoplastic cells in the aliquots compared to an untreated control HSC aliquot. The proportion of JAK2 V617F mutant cells, for example, can be measured by cell sorting (flow cytometry) and/or polymerase chain reaction (e.g., digital droplet polymerase chain reaction, ddPCR).

Hematopoietic stem cells (HSCs) from seven Polycythemia Vera (PV) patients were harvested. Some of the patients had already been treated with interferon-alpha while other patients had not (the “Never IFN” patients). The patient HSCs were a mixture of cells having JAK2 V617F mutations (myeloproliferative neoplastic stem cells (MPN-SCs)) and co-mingled HSPCs with only wild type JAK2 alleles (WT). Aliquots of the wild type and mutant MPN-SCs from each patient were treated with interferon-alpha and the ability of the cells to expand in the vascular niche conditions (endothelial feeder cells and low oxygen) was evaluated. Untreated cell expansion of the MPN-SCs and wild type cells from each patient was used as a control.

As shown in FIG. 10C, interferon-alpha (IFN) did reduce the expansion of some patients' MPN-stem cells relative to the normal HSPCs from the same patient (FIG. 10C, patients MPD377, MPD368, MPD536). These patients had achieved complete (IFN CHR) or partial (IFN PHR) to clinical use of IFN or had never been treated with IFN (Never IFN). However, interferon-alpha did not successfully reduce the expansion of all patients' MPN-SCs (FIG. 10C MPD090. MPD216, MPD472, MPD516). These resistant patients had evolved from PV to myelofibrosis on interferon (PV->MF) or had never been exposed to IFN as a therapy (Never IFN). Prior treatment of a patient did not influence the growth potential of some patients' MPN-SCs (compare. e.g., the expansion of MPN-SCs from patients MPD216/MPD516 (no effect) and MPD377 (MPN-SC targeted) in FIG. 10C).

These data indicate that treatment regimens are most effective when they are designed for the individual needs of a patient. The methods described herein provide in vitro cell culture screening methods that can quickly and effectively evaluate which therapeutic agents can most effectively be used to treat patients suffering from various diseases (e.g., cancer) and conditions (e.g., genetic conditions).

### Example 12: Multiplex Screening for Individualized Therapeutic Agents

This Example illustrates simultaneous evaluation of five different agents for identifying therapeutic agents for treatment of diseases and conditions.

Hematopoietic stem cells (HSCs) containing myeloproliferative neoplasm (MPN) stem and progenitor cells (MPN-SPCs) were harvest from six donors and cell types were separated. Cells were harvested from these donors by a simple blood draw making such study universally available and minimally-invasive for patient testing. The cell types tested were:
All hematopoietic cells in the culture (identified by CD45 staining; referred to as “Heme” cells);
CD45+CD34+ non-EC cells (referred to as “CD34” cells); and
CD31−CD45+CD34+CD38−CD45RA−CD90+ portion of the cells (referred to as HSC).
   Plates with 96 wells were prepared by addition of culture media and endothelial feeder cells. Aliquots of the different cell types were distributed into different wells of the 96-well plates. The following test agents were added to wells containing different cell types:
CPI-0610—a BET-domain inhibitor being used in clinical trials;
Ruxolitinib (Rux), an FDA approved JAK1/2 inhibitor used for PV and MF;
Pegylated interferon alpha (Pegasys; IFN), a standard of care off-label use agent for treatment of ET/PV and early MF;
CPI1205, an EZH2 inhibitor in clinical development; or
Combinations thereof.
   Five replicates were used for each agent for each donor cell type. The plates were incubated for 14 days at 37° C. in an atmosphere of 1% oxygen, and the cell expansion was evaluated for the different cell types incubated with the different test agents

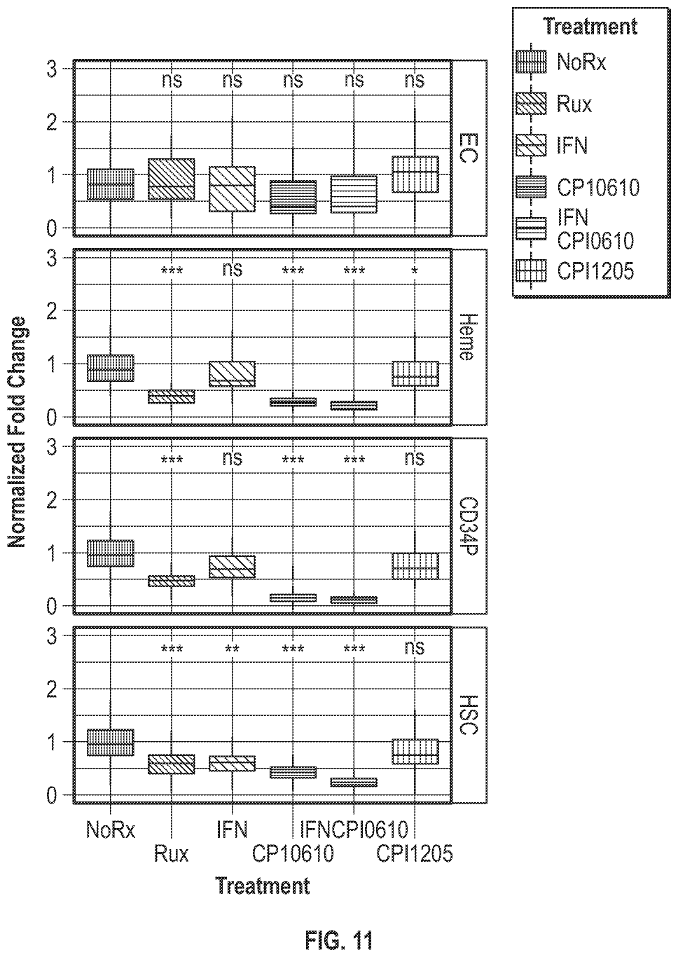

As shown in FIG. 11, several treatments significantly reduced the expansion of Heme, CD34, and HSC cell types without affecting the growth of the endothelial cells. For example, CPI-0610 and the combination of CPI-0610 and IFN was most effective at reducing expansion of the Heme, CD34, and HSC cell types

These data were obtained in less than two weeks, with little or no labor required during most of that time. The methods employed have been validated across more than seven orders of magnitude. And the additional replicates made possible by the scaled down and validated methods described herein provided scientifically superior results. This platform allows rapid screening of agents/combinations using very small numbers of cells and short culture periods and is without precedent for studies of malignant or normal hematopoietic stem cell biology and therapy.

### Example 13: HSC Growth/Harvesting with Dissolvable EC-Microcarriers

Fifteen different microcarriers were evaluated for speed of endothelial cell (EC) attachment, EC growth, EC viability, and EC function, once loaded with ECs using small scale cultures and using larger scale cultures in a bioreactor.

The types of microcarriers evaluated are listed in the following table.

| | | | SURFACE |
|---|---|---|---|
| MICROCARRIER | MANUFACTURER | MATRIX | COATING |
| CYTODEX 3 | GE Healthcare | Dextran | Type I |
| | | | porcine |
| | | | collagen |
| CULTISPHER-S | Percell-Biolytica | Type I | None |
| | | porcine | |
| | | gelatin | |
| FACT III | SoloHill | Polystyrene | Cationic |
| | | | Type I |
| | | | porcine |
| | | | collagen |
| COLLAGEN SH | SoloHill | Polystyrene | Type I |
| | | | porcine |
| | | | collagen |
| PRONECTIN-F | SoloHill | Polystyrene | Recombi- |
| | | | nant RGD |
| STAR-PLUS | SoloHill | Polystyrene | None? |
| PLASTIC | SoloHill | Polystyrene | None |
| PLASTIC-PLUS | SoloHill | Polystyrene | None |
| HILLEXII | SoloHill | Polystyrene | Cationic |
| | | | amine |
| POSITIVE | Corning | Polystyrene | Cationic |
| CHARGE | | | poly-D- |
| | | | lysine amine |
| ENHANCED | Corning | Polystyrene | Corning |
| ATTACHMENT | | | CellBIND |
| | | | surface |
| HIGH | Corning | Polystyrene | Synthemax |
| CONCENTRATION | | | II high con- |
| SYNTHEMAX II | | | centration |
| LOW | Corning | Polystyrene | Synthemax |
| CONCENTRATION | | | II low con- |
| SYNTHEMAX II | | | centration |
| UNTREATED | Corning | Polystyrene | None |
| DISSOLVABLE | Corning | Pectin | Synthemax |
| SYNTHEMAX II | | | II |

Only the Corning, pectin-based (Synthemax-II coated) and the dextran-based (CultiSpher-S) microcarriers were dissolvable microcarriers. Gamma-sterilized, GMP-grade Corning dissolvable microcarriers were available with cGLP certificates of analysis (CoA) and were selected for further study.

Varying conditions were studied to determine the best conditions for digestion and endothelial cell-harvest when using Corning SyntheMax dissolvable microcarriers. Pectinase (P) and EDTA (E) were required to digest the microcarrier polysaccharide matrix but use of pectinase and EDTA also was not sufficient for optimal cellular harvesting. Celase® (C; collagenase, neutral protease blend) and/or TripLE® (T; recombinant trypsin sold by ThermoFisher) were needed to digest cellular basement membranes. In addition, Benzonase® (B; endonuclease that degrades both DNA and RNA) was used to help disaggregate cells by digesting free nucleic acids.

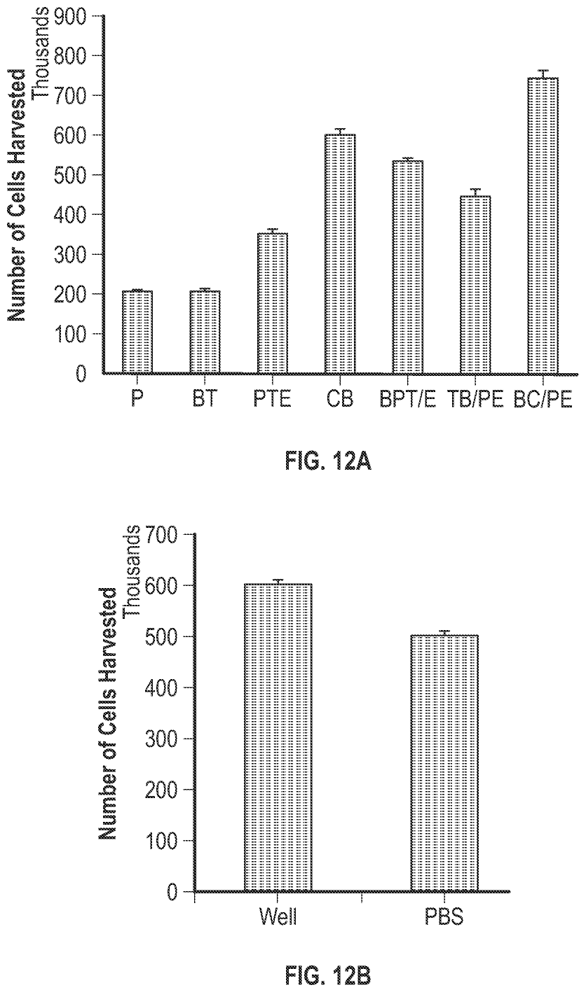

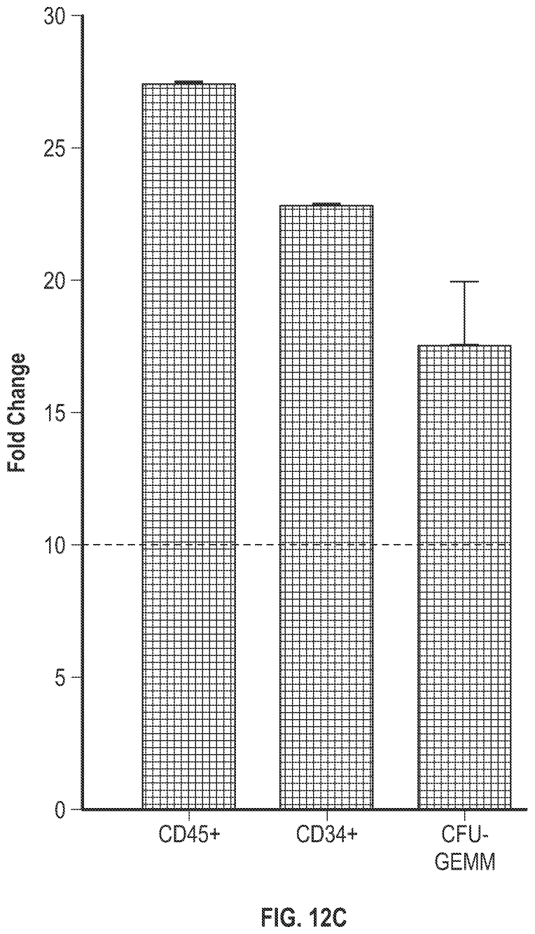

Optimal digestion and recovery of viable mono-cellular suspensions was obtained with pre-digestion of microcarriers by addition of Benzonase and Celase to cultures with stirring, followed by addition of EDTA and pectinase for digestion of the microcarriers. See FIG. 12A.

These and subsequent studies confirmed which harvest protocol was feasible in both in a microwell format and in the larger PBS Biotech vertical wheel format (70 mL scale). The optimized method reproducibly provided recovery of monocellular suspensions at all scales tested from both E4EC cultures and HSPC:E4EC cocultures (FIG. 12B).

In another experiment, using the methods described herein, Ad52-E4ECs were seeded onto Corning SyntheMax dissolvable microcarriers and hematopoietic stem/progenitor cells were added. This mixture was incubated under hypoxic conditions for 7 days.

When using the dissolvable microcarriers, hematopoietic cells with the following phenotypes were successfully expanded and then harvested: CD34+CD38−; CD45RA−; and CD49f+. FIG. 12C illustrates the expansion after harvesting of CD45+ and CD34+ cells as well as the ability of the harvested cell to form colony forming units that generated myeloid cells (CFU-GEMM). Neither the growth or the immunophenotypic profile of the expanded cells was compromised by digestion and cellular harvest from the microcarriers.

The harvested cells were then transplanted into NSG mice. Robust engraftment was confirmed. Subsequent studies using different seeding densities of HSPC:EC ratios on the Corning microcarriers demonstrated that robust expansion was routinely achieved for cells that were then capable of long-term NSG engraftment when the HSPC:EC ratio was maintained at less than 1 HSPC per 3 EC.

### Example 14: Expansion of Healthy AD-HSPCs Using EC-Seeded Corning Microcarriers

The following platforms were evaluated for use with microcarrier co-cultures: Spinner flask culture; WAVE bioreactor format (GE Healthcare Life Sciences); Stirred tank bioreactor (Eppendorf DasBox); and PBS Biotech vertical wheel bioreactor (PBS Biotech). The criteria for selection of platform for cellular expansion were as follows:
1) Ability to maintain hypoxic culture conditions;
2) Demonstrated reproducible, efficient seeding and pre-culture propagation of endothelial cells;
3) Scalable methods with minimal optimization over scale ranges of 1/30 to ¼;
4) Automated cGMP bioreactor available at full scale;
5) Demonstrated expansion of at least 10-fold or more for immature HSPCs in various scaled batches, with successful engraftment of the expanded cells in NSG mice.
Evaluations of the different platforms provided the following results.

### Spinner Flask

Initial suitability of a hypoxic, microcarrier based co-culture expansion platform was demonstrated and validated at the 70 mL scale using spinner flasks with disposable (Corning disposable) microcarriers. Adult human HSPCs expanded using Cultispher-S dissolvable microcarriers were shown to reproducibly expand >30-fold in 70 mL spinner flasks. The expanded HSPCs could successfully be cryopreserved, and the expanded HSPCs exhibited robust NSG engraftment compared to matched unexpanded HSPCs. However, no path for proportioned upscaling was available with the spinner flasks. It was anticipated that scale up would require re-optimization at each scale due to differences in hydrodynamics relating to differing vessel/baffle geometries. This platform remained a fall back if other approaches were unsuccessful.

### WAVE Bag

The WAVE bioreactor format was tested using the CultiSpher-S microcarrier. Several aspects of the platform were problematic.
Lack of validated small-scale (<600 mL) culture bags for testing & optimization.
Lack of controller sufficient to maintain hypoxic condition within the culture bag. Controller could not regulate adequately regulate oxygen tension.
Definition of platform motion to suspend microcarriers for seeding E4ECs was difficult.
   Due to these and other problems, preliminary testing in large culture bags did not yield viable E4ECs after attempted seeding onto microcarriers. For these reasons, preliminary feasibility could not be demonstrated and further development of a WAVE platform was halted. This approach was problematic and no further development efforts were made since the WAVE/Xuri system tested could not support the requisite E4EC seeding steps.

### Stirred Tank Bioreactor

The DasBox platform (Eppendorf) was geometrically scalable, fully automated and capable of maintaining hypoxic conditions during culture. However, preliminary evaluation of the DasBox bioreactor system (stirred tank) at the 70 mL scale identified critical initial problems. Difficulties were encountered in maintaining suspension of microcarriers (CultiSpher-S). The impeller speed and resultant shear forces required to suspend these large microcarriers was too high to support adequate E4EC seeding and growth. Although this problem could potentially be overcome—either by using an alternative, available impeller design or smaller, GMP microcarriers that consequently have both a lower settling time and lower impeller shear required to loft microcarriers-further pilot testing was halted in the DasBox bioreactor because preliminary results from the PBS Biotech vertical wheel bioreactor were highly promising.

### PBS Biotech Vertical Wheel Bioreactor

Significant proof-of-principle was successfully achieved by the inventors using the PBS Biotech Vertical Wheel bioreactor platform. PBS Biotech family of bioreactors includes a small scale PBSmini base unit and disposable, 100 mL (PBS 0.1 L) and 500 mL (0.5 L) single use culture vessels. Tests indicated that the PBS format has several appealing features:
1) Fully geometrically scaled vessel designs with shared hydrodynamics to enable predictable upscaling;
2) Availability of vessels in several scales that permit modest pilot scale optimization (60 mL), upscale testing to 500 mL with predictable upscaling to 3+ Liter bioreactors;
3) Cost effective vessels at all scales for testing and production;
4) Availability of fully cGMP monitored and microprocessor controlled bioreactor;
5) Capability to maintain hypoxic conditions throughout culture period;
6) Low shear vertical wheel impeller capable of lofting microcarriers.
   Successful incremental scale-up studies were completed using the PBS Biotech Vertical Wheel Bioreactor and at each stage, multidimensional flow cytometry to enumerate cells with immunophenotype of immature HSPCs, in vitro CFU-GEMM read out and, at critical points, 16+ week NSG engraftment have been used to confirm successful scale-up in the platform.

Testing of the PBS Biotech Vertical Wheel Bioreactor demonstrated the following:
1) Robust (20-80 fold) expansion was achieved of human adult HSPCs with retention of immature immunophenotypes and functions (CFU-GEMM) at the 60 mL scale.
2) Reproducible EC seeding and expansion at 60 and 100 mL and 500 mL scale using two different dissolvable microcarriers (Cultispher-S and Corning Synthemax dissolvable).
3) Cell harvest procedures for ECs and EC:HSPC co-culture products could readily be optimized using cGMP reagents to generate high-viability mono-cellular suspensions free of residual microcarriers.
4) Reproducible seeding and expansion of ECs:HSPC co-cultures was achieved at 100 mL scale.
5) GMP standard operating procedures (SOP) were developed for EC culture and for HSPC expansion using EC-loaded microcarriers.
   Therefore, the methods described herein can provide expansion of HSPCs beyond what is currently available.

### REFERENCES

1. Lubeck, D., et al. Estimated Life Expectancy and Income of Patients With Sickle Cell Disease Compared With Those Without Sickle Cell Disease. JAMA Netw Open 2, e1915374 (2019).
2. Hsieh, M. M., Fitzhugh, C. D. & Tisdale, J. F. Allogeneic hematopoietic stem cell transplantation for sickle cell disease: the time is now. Blood 118, 1197-1207 (2011).
3. Darbari, D. S., et al. Pain and opioid use after reversal of sickle cell disease following HLA-matched sibling haematopoietic stem cell transplant. Br J Haematol 184, 690-693 (2019).
4. Naldini, L. Gene therapy returns to centre stage. Nature 526, 351-360 (2015).
5. Zonari, E., et al. Efficient Ex Vivo Engineering and Expansion of Highly Purified Human Hematopoictic Stem and Progenitor Cell Populations for Gene Therapy. Stem Cell Reports 8,977-990 (2017).
6. Morrison, S. J. & Scadden. D. T. The bone marrow niche for haematopoietic stem cells. Nature 505, 327-334 (2014).
7. Lis, R., et al. Conversion of adult endothelium to immunocompetent haematopoietic stem cells. Nature 545, 439-445 (2017).
8. Ding, L., Saunders, T. L., Enikolopov, G. & Morrison, S. J. Endothelial and perivascular cells maintain haematopoietic stem cells. Nature 481, 457-462 (2012).
9. Butler. J. M., et al. Endothelial cells are essential for the self-renewal and repopulation of Notch-dependent hematopoietic stem cells. Cell stem cell 6, 251-264 (2010).
10. Butler, J. M., et al. Development of a vascular niche platform for expansion of repopulating human cord blood stem and progenitor cells. Blood 120, 1344-1347 (2012).
11. Seandel, M., et al. Generation of a functional and durable vascular niche by the adenoviral E4ORF1 gene. Proc Natl Acad Sci USA 105, 19288-19293 (2008).
12. Boitano, A. E., et al. Aryl hydrocarbon receptor antagonists promote the expansion of human hematopoietic stem cells. Science 329, 1345-1348 (2010).
13. Fares, I., et al. Cord blood expansion. Pyrimidoindole derivatives are agonists of human hematopoietic stem cell self-renewal. Science 345, 1509-1512 (2014).
14. Gu, A., et al. Engraftment and lineage potential of adult hematopoietic stem and progenitor cells is compromised following short-term culture in the presence of an aryl hydrocarbon receptor antagonist. Hum Gene Ther Methods 25, 221-231 (2014).
15. Chou, S., Chu, P., Hwang, W. & Lodish, H. Expansion of human cord blood hematopoietic stem cells for transplantation. Cell stem cell 7, 427-428 (2010).
16. Singh, K. P., Garrett, R. W., Casado, F. L. & Gasiewicz, T. A. Aryl hydrocarbon receptor-null allele mice have hematopoietic stem/progenitor cells with abnormal characteristics and functions. Stem cells and development 20, 769-784 (2011).
17. Singh, K. P., et al. Loss of aryl hydrocarbon receptor promotes gene changes associated with premature hematopoietic stem cell exhaustion and development of a myeloproliferative disorder in aging mice. Stem cells and development 23, 95-106 (2014).
18. Gasiewicz, T. A., Singh, K. P. & Bennett, J. A. The Ah receptor in stem cell cycling, regulation, and quiescence. Annals of the New York Academy of Sciences (2014).
19. Simsek. T., et al. The distinct metabolic profile of hematopoietic stem cells reflects their location in a hypoxic niche. Cell stem cell 7, 380-390 (2010).
20. Takubo, K., et al. Regulation of the HIF-1alpha level is essential for hematopoietic stem cells. Cell stem cell 7, 391-402 (2010).
21. Suda, T., Takubo, K. & Semenza, G. L. Metabolic regulation of hematopoietic stem cells in the hypoxic niche. Cell stem cell 9, 298-310 (2011).
22. Parmar, K., Mauch, P., Vergilio, J. A., Sackstein, R. & Down. J. D. Distribution of hematopoietic stem cells in the bone marrow according to regional hypoxia. Proc Natl Acad Sci USA 104, 5431-5436 (2007).
23. Spencer, J. A., et al. Direct measurement of local oxygen concentration in the bone marrow of live animals. Nature 508, 269-273 (2014).
24. von Laer, D., et al. Loss of CD38 antigen on CD34+CD38+ cells during short-term culture. Leukemia 14.947-948 (2000).
25. Ngom, M., et al. UM171 Enhances Lentiviral Gene Transfer and Recovery of Primitive Human Hematopoietic Cells. Mol Ther Methods Clin Dev 10, 156-164 (2018).
26. Lidonnici, M. R., et al. Plerixafor and G-CSF combination mobilizes hematopoietic stem and progenitors cells with a distinct transcriptional profile and a reduced in vivo homing capacity compared to plerixafor alone. Haematologica 102, e120-e124 (2017).
27. Kalina, T., et al. EuroFlow standardization of flow cytometer instrument settings and immunophenotyping protocols. Leukemia 26, 1986-2010 (2012).
28. Juntilla, M. M., et al. AKT1 and AKT2 maintain hematopoietic stem cell function by regulating reactive oxygen species. Blood 115, 4030-4038 (2010).
29. Ito, K., et al. Reactive oxygen species act through p38 MAPK to limit the lifespan of hematopoietic stem cells. Nat Med 12, 446-451 (2006).
30. Mah, L. J., El-Osta, A. & Karagiannis, T. C. gammaH2AX: a sensitive molecular marker of DNA damage and repair. Leukemia 24, 679-686 (2010).
31. Eliasson, P., et al. Hypoxia mediates low cell-cycle activity and increases the proportion of long-term-reconstituting hematopoietic stem cells during in vitro culture. Experimental hematology 38, 301-310 e302 (2010).
32. Murray, J. K., et al. Dipeptidyl-quinolone derivatives inhibit hypoxia inducible factor-1alpha prolyl hydroxylases-1, -2, and -3 with altered selectivity. J Comb Chem 12, 676-686 (2010).
33. Ellinghaus, P., et al. BAY 87-2243, a highly potent and selective inhibitor of hypoxia-induced gene activation has antitumor activities by inhibition of mitochondrial complex I. Cancer Med 2, 611-624 (2013).
34. Kobayashi, H., et al. Angiocrine factors from Akt-activated endothelial cells balance self-renewal and differentiation of haematopoietic stem cells. Nat Cell Biol 12, 1046-1056 (2010).
35. Alhamdoosh, M., et al. Combining multiple tools outperforms individual methods in gene set enrichment analyses. Bioinformatics 33, 414424 (2017).
36. Goldman, D. C., et al. BMP4 regulates the hematopoietic stem cell niche. Blood 114, 4393-4401 (2009).
37. Catlin, S. N., Busque. L., Gale, R. E., Guttorp, P. & Abkowitz, J. L. The replication rate of human hematopoietic stem cells in vivo. Blood 117, 4460-4466 (2011).
38. Levesque, J. P., Hendy, J., Takamatsu, Y., Simmons, P. J. & Bendall, L. J. Disruption of the CXCR4/CXCL12 chemotactic interaction during hematopoietic stem cell mobilization induced by GCSF or cyclophosphamide. J Clin Invest 111, 187-196 (2003).
39. Manci, E. A., et al. Perivascular fibrosis in the bone marrow in sickle cell disease. Arch Pathol Lab Med 128, 634-639 (2004).
40. Ribeil, J. A., et al. Gene Therapy in a Patient with Sickle Cell Disease. N Engl J Med 376, 848-855 (2017).
41. Lagresle-Peyrou, C., et al. Plerixafor enables safe, rapid, efficient mobilization of hematopoietic stem cells in sickle cell disease patients after exchange transfusion. Haematologica 103, 778-786 (2018).
42. Boulad, F., et al. Safety and efficacy of plerixafor dose escalation for the mobilization of CD34(+) hematopoietic progenitor cells in patients with sickle cell disease: interim results. Haematologica 103, 770-777 (2018).
43. Bai, T., et al. Expansion of primitive human hematopoietic stem cells by culture in a zwitterionic hydrogel. Nat Med 25, 1566-1575 (2019).
44. Mantel, C. R., et al. Enhancing Hematopoietic Stem Cell Transplantation Efficacy by Mitigating Oxygen Shock. Cell 161, 1553-1565 (2015).
45. Jackson. C. S., et al. Targeting the aryl hydrocarbon receptor nuclear translocator complex with DMOG and Stemregenin 1 improves primitive hematopoietic stem cell expansion. Stem Cell Res 21, 124-131 (2017).
46. Ding, L. & Morrison, S. J. Haematopoietic stem cells and early lymphoid progenitors occupy distinct bone marrow niches. Nature 495, 231-235 (2013).
47. Delaney, C., et al. Notch-mediated expansion of human cord blood progenitor cells capable of rapid myeloid reconstitution. Nat Med 16, 232-236 (2010).
48. Csaszar, E., et al. Rapid expansion of human hematopoietic stem cells by automated control of inhibitory feedback signaling. Cell stem cell 10, 218-229 (2012).
49. de Lima, M., et al. Transplantation of ex vivo expanded cord blood cells using the copper chelator tetraethylenepentamine: a phase I/II clinical trial. Bone marrow transplantation 41, 771-778 (2008).
50. Antonchuk, J., Sauvageau, G. & Humphries. R. K. HOXB4-induced expansion of adult hematopoietic stem cells ex vivo. Cell 109, 39-45 (2002).
51. Grosse. S. D., et al. Sickle cell disease in Africa: a neglected cause of early childhood mortality. American journal of preventive medicine 41, S398-405 (2011).
52. Grosse, S. D., et al. The Jamaican historical experience of the impact of educational interventions on sickle cell disease child mortality. American journal of preventive medicine 42, e101-103 (2012).
53. Liberzon, A., et al. The Molecular Signatures Database (MSigDB) hallmark gene set collection. Cell Syst 1, 417-425 (2015).
54. Seandel, M., et al. Generation of a functional and durable vascular niche by the adenoviral E4ORF1 gene. Proc Natl Acad Sci USA 105, 19288-19293 (2008).
55. Alter, B. P. et al. Malignancies and survival patterns in the National Cancer Institute inherited bone marrow failure syndromes cohort study. Br J Haematol 150, 179-188, (2010).
56. Butturini, A. et al. Hematologic abnormalities in Fanconi anemia: an International Fanconi Anemia Registry study. Blood 84, 1650-1655 (1994).
57. Cioc, A. M., Wagner, J. E., MacMillan, M. L., DeFor, T. & Hirsch. B. Diagnosis of myelodysplastic syndrome among a cohort of 119 patients with fanconi anemia: morphologic and cytogenetic characteristics. Am J Clin Pathol 133, 92-100 (2010).
58. Rio, P. et al. Engraftment and in vivo proliferation advantage of gene-corrected mobilized CD34(+) cells from Fanconi anemia patients. Blood 130, 1535-1542, (2017).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various features of the invention according to the foregoing description provided in the specification and figures.

### Statements

1. A method comprising expanding hematopoietic stem cells and/or hematopoietic progenitor cells ex vivo under conditions comprising reduced atmospheric oxygen levels or reduced reactive oxygen species (ROS) levels in the presence of endothelial cells, to thereby generate an expanded population of hematopoietic stem cells and/or hematopoietic progenitor cells.
2. The method of statement 1, wherein the reduced atmospheric oxygen levels comprise an atmosphere of less than 5% oxygen, or less than 4% oxygen, or less than 3% oxygen, or less than 2% oxygen, or less than 1% oxygen, or less than 0.5% oxygen, or less than 0.1% oxygen, or less than 0.05% oxygen, or less than 0.01% oxygen.
3. The method of statement 1 or 2, wherein the reduced atmospheric oxygen levels comprise an atmosphere of less than or about 1% oxygen.
4. The method of any one of statements 1-3, wherein the conditions comprise a culture medium that supports the growth of the hematopoietic stem cells and/or hematopoietic progenitor cell.
5. The method of any one of statements 1-4, wherein the reduced reactive oxygen species (ROS) levels comprise at least one reactive oxygen species (ROS) scavenger in a culture medium that supports the growth of the hematopoietic stem cells and/or hematopoietic progenitor cell.
6. The method of statement 5, wherein the at least one reactive oxygen species (ROS) scavenger is N-acetylcysteine (NAC), sodium pyruvate, N,N′-dimethylthiourea (DMTU), Trolox (6-hydroxy-2,5,7,8-tetramethylchroman-2-carboxylic acid), α-tocopherol, Ebselen (2-phenyl-1,2-benzisoselenazol-3(2H)-one), uric acid, vitamin C (ascorbic acid), L-galactonic acid-g-galactose, imidazole, MnTBAP (manganese(III)-tetrakis(4-benzoic acid)porphyrin), or combinations thereof.
7. The method of any one of statements 4-6, wherein the culture medium further comprises at least one cytokine.
8. The method of statement 7, wherein the at least one cytokine is FGF2, IGF. EGF, VEGF, or a combination thereof.
9. The method of statement 7 or 8, wherein the at least one cytokine is FGF2.
10. The method of any one of statements 4-9, wherein the culture medium further comprises heparin.
11. The method of any one of statements 1-10, wherein the conditions comprise reduced reactive oxygen species (ROS) and a normoxic atmosphere.
12. The method of statement 11, wherein the normoxic atmosphere comprises oxygen levels of more than 3%.
13. The method of any one of statements 4-12, wherein the culture medium further comprises a TGFβ inhibitor, a TGFβ signaling inhibitor, a chemokine, a hypoxia mimetic, a ROS scavenger, a HIF stabilizer, an mTOR inhibitor, or a combination thereof.
14. The method of any one of statements 4-13, further comprising BMP4.
15. The method of statement 13 or 14, wherein the chemokine is CXCL12 (SDF1.
16. The method of any one of statements 13-15, wherein the HIF stabilizer is selected from the group consisting of Roxadustat (RXD) (FG-4592), Vadadustat (AKB-6548), Daprodustat (GSK-1278863), and Molidustat (BAY 85-3934).
17. The method of any one of statements 13-16, wherein the hypoxia mimetic is desferrioxamine (DFO).
18. The method of any one of statements 13-17, wherein the ROS scavenger is selected from the group consisting of N-acetyl-L-cysteine (NAC), Sodium pyruvate, N,N′-dimethylthiourea (DMTU), Trolox (6-hydroxy-2,5,7,8-tetramethylchroman-2-carboxylic acid), α-tocopherol, Ebselen (2-phenyl-1,2-benzisoselenazol-3(2H)-one), uric acid, Vitamin C, and MnTBAP (manganese(III)-tetrakis(4-benzoic acid)porphyrin).
19. The method of any one of statements 13-18, wherein the mTOR inhibitor is selected from the group consisting of rapamycin, temsirolymus, everolimus, ridaforolimus, Dactolisib (NVP-BEZ235), Omipalisib (GSK2126458), and sapanisertib.
20. The method of any one of statements 13-19, wherein the TGFβ signaling inhibitor inhibits TGFβ signaling through a TGFβ receptor that is TGFβR2 or ALK5 (TGFβR1).
21. The method of any one of statements 13-20, wherein the TGFβ signaling inhibitor is an antibody which antagonizes the interaction and binding between TGFβ and TGFβ receptor or an anti-TGFβ-1,2,3 monoclonal antibody (e.g., 1D11.16.8).
22. The method of any one of statements 13-21, wherein the TGFβ signaling inhibitor is a soluble polypeptide composed of the extracellular domain of a TGFβ receptor.
23. The method of any one of statements 13-22, wherein the TGFβ signaling inhibitor is an oligonucleotide selected from the group consisting of an antisense. RNAi, dsRNA, siRNA and ribozyme molecule.
24. The method of any one of statements 13-23, wherein said TGFβ signaling inhibitor is a small molecule organic compound.
25. The method of any one of statements 13-24, wherein said TGFβ signaling inhibitor antagonizes the activation of latent TGFβ to its active form capable of activating signaling via a TGFβ receptor.
26. The method of any of any one of statements 1-25, wherein the endothelial cells are grown and used within microcarriers.
27. The method of statement 26, wherein the microcarriers are comprised of DEAE-dextran, glass, polystyrene plastic, acrylamide, collagen, pectin, or alginate.
28. The method of statement 26, wherein the microcarriers are dissolvable or digestible (e.g., by enzymes) without adversely affecting the endothelial cells or the hematopoietic stem cells and/or the hematopoietic progenitor cells.
29. The method of any of statements 1-28, wherein the endothelial cells are not genetically modified human endothelial cells.
30. The method of any of statements 1-28, wherein the endothelial cells express an adenovirus E4 open reading frame 1 (E4ORF1) polypeptide.
31. The method of any one of statements 1-28, or 30, wherein the endothelial cells express an adenovirus E4 open reading frame 1 (E4ORF1) polypeptide comprising any of SEQ ID NOs: 3, 5, 7, 9, 12, or 14.
32. The method of any one of statements 1-28, 30, or 31, wherein the endothelial cells comprise an adenovirus E4 open reading frame (E4ORF1) nucleic acid with a sequence comprising any of SEQ ID NO:1, 2, 4, 6, 8, 11, or 13.
33. The method of any one of statements 1-28, 30-32, wherein the endothelial cells express an Akt gene from an exogenous expression cassette or exogenous expression vector.
34. The method of any one of statements 1-33, wherein the hematopoietic stem cells and/or hematopoietic progenitor cells are human hematopoietic stem cells and/or human hematopoietic progenitor cells.
35. The method of any one of statements 1-34, wherein the hematopoietic stem cells and/or hematopoietic progenitor cells are obtained from donor bone marrow or donor peripheral blood.
36. The method of any one of statements 1-35, wherein the hematopoietic stem cells and/or hematopoietic progenitor cells are obtained by apheresis blood cell collection or from umbilical cord blood of a human donor.
37. The method of any one of statements 1-36, wherein the hematopoietic stem cells and/or hematopoietic progenitor cells are expanded by 2 fold, 3 fold, 4 fold, 5 fold, 6 fold, 7 fold, 8 fold, 9 fold, 10 fold, 12 fold, 15 fold, 20 fold, 40 fold, or more.
38. The method of any one of statements 1-37, wherein the hematopoietic stem cells and/or hematopoietic progenitor cells are expanded by at least 10-fold, or at least 15-fold, or at least 20-fold, or at least 30-fold, or at least 40-fold, or at least 50 fold.
39. The method of any one of statements 1-38, wherein the expanded population of hematopoietic stem cells and/or hematopoietic progenitor cells is from a single cell or about 1-1000 cells.
40. The method of statement 39, wherein the single cell or the 1-1000 cells is expanded into a population of at least 5 million, at least 10 million, or at least 20 million, or at least 30 million, or at least 40 million, or at least 50 million hematopoietic stem cells and/or hematopoietic progenitor cells.
41. The method of any one of statements 1-40, wherein diseased or mutant hematopoietic stem cells and/or hematopoietic progenitor cells are expanded to the same extent as healthy or wild type hematopoietic stem cells and/or hematopoietic progenitor cells.
42. The method of any one of statements 1-41, further comprising administering the expanded population of hematopoietic stem cells and/or hematopoietic progenitor cells to a subject.
43. The method of any one of statements 1-42, wherein the hematopoietic stem cells and/or hematopoietic progenitor cells are from a subject suffering from a disease or condition (i.e., the donor is the same as the subject).
44. The method of any one of statements 42-43, wherein the subject suffers from a disease caused by a genetic defect that affects hematopoietic cells, a metabolic condition, an infection of hematopoietic cells, or a reaction to a drug.
45. The method of any one of statements 42-44, wherein the subject suffers from Fanconi Anemia (FA), sickle cell anemia, severe combined immunodeficiency, β-thalassemia, Wiskott-Aldrich syndrome, or leukodystrophy.
46. The method of any one of statements 1-45, further comprising genetically modifying the hematopoietic stem cells and/or hematopoietic progenitor cells to generate genetically modified hematopoietic stem cells and/or hematopoietic progenitor cells, and then expanding the genetically modified hematopoietic stem cells and/or hematopoietic progenitor cells.
47. The method of statement 46, wherein genetically modifying the hematopoietic stem cells and/or hematopoietic progenitor cells obviates or alleviates the genetic defect.
48. The method of any one of statements 1-47, wherein the expanded population of hematopoietic stem cells and/or hematopoietic progenitor cells administered to the subject are an expanded population of autologous hematopoietic stem cells and/or autologous hematopoietic progenitor cells.
49. The method of any one of statements 42-48, wherein administering the expanded population of hematopoietic stem cells and/or hematopoietic progenitor cells to the subject alleviates symptoms of the disease or the condition.
50. The method of any one of statements 1-49, further comprising aliquoting portions of the hematopoietic stem cells and/or hematopoietic progenitor cells into a series of separate receptacles, adding one or more different test agents to the separate receptacles to form a series of test mixtures, expanding the hematopoietic stem cells and/or hematopoietic progenitor cells in the test mixtures, and quantifying the cells within the test mixtures after expansion to obtain a series of quantified cell numbers for the different test mixtures.
51. The method of statement 50, further comprising comparing each of the series of quantified cell numbers with a cell number control.
52. The method of statement 50, wherein the cell number control comprises an average quantified control cell number.
53. The method of statement 50, wherein the average quantified control cell number is the average number of cells expanded in one or more control receptacles comprising the hematopoietic stem cells and/or hematopoietic progenitor cells without a test agent.
54. The method of any one of statements 50-53, wherein the hematopoietic stem cells and/or hematopoietic progenitor cells are from a donor or subject with a disease or condition.
55. The method of statement 54, wherein the disease or condition is cancer.
56. A composition comprising a population of hematopoietic stem cells and/or hematopoietic progenitor cells expanded by the method of any one of statements 1-55.
57. A composition comprising a population of hematopoietic stem cells and/or hematopoietic progenitor cells in a solution comprising less than 1% molecular oxygen, or less than 0.5% molecular oxygen, or less than 0.25% molecular oxygen, or less than molecular 0.2% oxygen, or less than molecular 0.1% oxygen, or less than molecular 0.05% oxygen, or less than molecular 0.01% oxygen.
58. The composition of statement 56 or 57, further comprising a TGFβ inhibitor of a TGFβ family member, a TGFβ signaling inhibitor, a chemokine, a hypoxia mimetic, a ROS scavenger, a HIF stabilizer, an mTOR inhibitor, or a combination thereof.
59. The composition of any one of statements 56-58, further comprising BMP4.
60. The composition of statement 58 or 59, wherein the chemokine is CXCL12 (SDF1).
61. The composition of any one of statements 58-60, wherein the HIF stabilizer is selected from the group consisting of Roxadustat (RXD) (FG-4592), Vadadustat (AKB-6548), Daprodustat (GSK-1278863), and Molidustat (BAY 85-3934).
62. The composition of any one of statements 58-61, wherein the hypoxia mimetic is desferrioxamine (DFO).
63. The composition of any one of statements 58-62, wherein the ROS scavenger is N-acetylcysteine (NAC), Sodium pyruvate, N,N′-dimethylthiourea (DMTU), Trolox (6-hydroxy-2,5,7,8-tetramethylchroman-2-carboxylic acid), α-tocopherol, Ebselen (2-phenyl-1,2-benzisoselenazol-3(2H)-one), uric acid, Vitamin C, MnTBAP (manganese(III)-tetrakis(4-benzoic acid)porphyrin), or a combination thereof.
64. The composition of any one of statements 58-63, wherein the mTOR inhibitor is rapamycin, temsirolymus, everolimus, ridaforolimus, Dactolisib (NVP-BEZ235), Omipalisib (GSK2126458), sapanisertib, or a combination thereof.
65. The composition of any one of statements 58-64, wherein the TGFβ signaling inhibitor inhibits TGFβ signaling through a TGFβ receptor that is TGFβR2 or ALK5 (TGFβR1).
66. The composition of any one of statements 58-65, wherein the TGFβ signaling inhibitor is an antibody which antagonizes the interaction and binding between TGFβ and TGFβ receptor or an anti-TGFβ-1,2,3 monoclonal antibody (e.g., 1D11.16.8).
67. The composition of any one of statements 58-66, wherein the TGFβ signaling inhibitor is a soluble polypeptide composed of the extracellular domain of a TGFβ receptor.
68. The composition of any one of statements 58-67, wherein the TGFβ signaling inhibitor is an oligonucleotide selected from the group consisting of an antisense, RNAi, dsRNA, siRNA and ribozyme molecule.
69. The composition of any one of statements 58-68, wherein said TGFβ signaling inhibitor is a small molecule organic compound.
70. The composition of any one of statements 58-69, wherein said TGFβ signaling inhibitor antagonizes the activation of latent TGFβ to its active form capable of activating signaling via a TGFβ receptor.
71. The composition of any one of statements 56-70, comprising at least 10⁶ cells, or at least 10⁷ cells, or at least 10⁸ cells, or at least 10⁹ cells, or at least 10¹⁰ cells, or at least 10¹¹ cells, or at least 10¹² cells.
72. The composition of any one of statements 56-71, wherein the hematopoietic stem cells and/or hematopoietic progenitor cells are expanded from a single donor or person.

### Method Statement Set 1

1. A method for expanding hematopoietic stem cells (HSCs), comprising culturing the HSCs under a hypoxic condition and in the presence of endothelial feeder cells.
2. The method of statement 1, wherein the hypoxic condition comprises an oxygen level of 3% or less.
3. The method of statement 1, wherein the endothelial feeder cells are human umbilical vascular endothelial cells (HUVECs).
4. The method of statement 1, wherein the endothelial feeder cells do not express the adenovirus E4 open reading frame 1 (E4ORF1) gene, and wherein the HSCs and the endothelial feeder cells are cultured in a medium that comprises a cytokine.
5. The method of statement 4, wherein the cytokine is selected from the group consisting of FGF2, IGF, EGF, VEGF, and a combination thereof.
6. The method of statement 5, wherein the medium further comprises heparin.
7. The method of statement 4, wherein the cytokine is FGF2.
8. The method of statement 7, wherein the medium comprises FGF2 and one or more of IGF, EGF, or VEGF.
9. The method of statement 1, wherein the endothelial feeder cells express the adenovirus E4 open reading frame 1 (E4ORF1) gene.
10. The method of statement 1, wherein the HSCs are obtained from the bone marrow or blood (such as apheresis blood cell collection or umbilical cord blood) of a human subject.
11. The method of statement 10, wherein the human subject suffers from a disease caused by a genetic defect that affects hematopoietic cells, for example, Fanconi Anemia (FA), sickle cell anemia, severe combined immunodeficiency, β-thalassemia, Wiskott-Aldrich syndrome, and leukodystrophy.
12. The method of statement 1, wherein the HSCs are expanded by 2 fold, 3 fold, 4 fold or more.

### Method Statement Set 2

1. A method for expanding hematopoietic stem cells (HSCs), comprising culturing the HSCs under normoxic conditions, in the presence of endothelial feeder cells, and in a culturing medium that comprises a factor selected from the group consisting of a TGFβ family member inhibitor. TGFβ signaling inhibitor, a chemokine, a hypoxia mimetic, a ROS scavenger, a HIF stabilizer, and an mTOR inhibitor.
2. The method of statement 1, wherein the normoxic condition comprises an oxygen level of more than 3%.
3. The method of statement 1, wherein the endothelial feeder cells express the adenovirus E4 open reading frame 1 (E4ORF1) gene or an Akt gene from an exogenous vector.
4. The method of statement 3, wherein the endothelial feeder cells are human umbilical vascular endothelial cells (HUVECs).
5. The method of statement 1, further comprising BMP4.
6. The method of statement 1, wherein the chemokine is CXCL12 (SDF1).
7. The method of statement 1, wherein the HIF stabilizer is selected from the group consisting of Roxadustat (RXD) (FG-4592), Vadadustat (AKB-6548). Daprodustat (GSK-1278863), and Molidustat (BAY 85-3934).
8. The method of statement 1, wherein the hypoxia mimetic is desferrioxamine (DFO).
9. The method of statement 1, wherein the ROS scavenger is selected from the group consisting of N-acetyl-L-cysteine (NAC), Sodium pyruvate, N,N′-dimethylthiourea (DMTU). Trolox (6-hydroxy-2,5,7,8-tetramethylchroman-2-carboxylic acid), α-tocopherol, Ebselen (2-phenyl-1,2-benzisoselenazol-3(2H)-one), uric acid, Vitamin C, and MnTBAP (manganese(III)-tetrakis(4-benzoic acid)porphyrin).
10. The method of statement 1, wherein the mTOR inhibitor is selected from the group consisting of rapamycin, temsirolymus, everolimus, ridaforolimus, Dactolisib (NVP-BEZ235), Omipalisib (GSK2126458), and sapanisertib.
11. The method of statement 1, wherein the TGFβ signaling inhibitor inhibits TGFβ signaling through a TGFβ receptor that is TGFβR2 or ALK5 (TGFβR1).
12. The method of statement 1, wherein the TGFβ signaling inhibitor is an antibody which antagonizes the interaction and binding between TGFβ and TGFβ receptor.
13. The method of statement 1, wherein the TGFβ signaling inhibitor is a soluble polypeptide composed of the extracellular domain of a TGFβ receptor.
14. The method of statement 1, wherein the TGFβ signaling inhibitor is an oligonucleotide selected from the group consisting of an antisense, RNAi, dsRNA, siRNA and ribozyme molecule.
15. The method of statement 1, wherein said TGFβ signaling inhibitor is a small molecule organic compound.
16. The method of statement 1, wherein said TGFβ signaling inhibitor antagonizes the activation of latent TGFβ to its active form capable of activating signaling via a TGFβ receptor.
17. The method of statement 1, wherein the HSCs are obtained from the bone marrow or blood (e.g., umbilical cord blood) of a human subject.
18. The method of statement 17, wherein the human subject suffers from a disease caused by a genetic defect that affects hematopoietic cells, for example, Fanconi Anemia (FA), sickle cell anemia, severe combined immunodeficiency, β-thalassemia, Wiskott-Aldrich syndrome, and leukodystrophy.
19. The method of statement 1, wherein the HSCs are expanded by 2 fold, 3 fold, 4 fold or more.

### Method Statement Set 3

1. A method for expanding hematopoietic stem cells (HSCs) comprising culturing the HSCs under normoxic conditions in the absence of endothelial feeder cells, wherein the culturing medium comprises a factor selected from the group consisting of a TGF signaling inhibitor, a hypoxia mimetic, a HIF stabilizer, a ROS scavenger, and an mTOR inhibitor.
2. The method of statement 1, wherein the HIF stabilizer is selected from the group consisting of Roxadustat (RXD) (FG-4592), Vadadustat (AKB-6548), Daprodustat (GSK-1278863), and Molidustat (BAY 85-3934).
3. The method of statement 1, wherein the hypoxia mimetic is desferrioxamine (DFO).
4. The method of statement 1, wherein the ROS scavenger is selected from the group consisting of N-acetyl-L-cysteine (NAC), Sodium pyruvate, N,N′-dimethylthiourea (DMTU), Trolox (6-hydroxy-2,5,7,8-tetramethylchroman-2-carboxylic acid), α-tocopherol, Ebselen (2-phenyl-1,2-benzisoselenazol-3(2H)-one), uric acid, Vitamin C, and MnTBAP (manganese(III)-tetrakis(4-benzoic acid)porphyrin).
5. The method of statement 1, wherein the mTOR inhibitor is selected from the group consisting of rapamycin, temsirolymus, everolimus, ridaforolimus, Dactolisib (NVP-BEZ235), Omipalisib (GSK2126458), and sapanisertib.
6. The method of statement 1, wherein the TGFβ signaling inhibitor inhibits TGFβ signaling through a TGFβ receptor that is TGF R2 or ALK5 (TGFβR1).
7. The method of statement 1, wherein the TGFβ signaling inhibitor is an antibody which antagonizes the interaction and binding between TGFβ and TGFβ receptor.
8. The method of statement 1, wherein the TGFβ signaling inhibitor is a soluble polypeptide composed of the extracellular domain of a TGFβ receptor.
9. The method of statement 1, wherein the TGFβ signaling inhibitor is an oligonucleotide selected from the group consisting of an antisense, RNAi, dsRNA, siRNA and ribozyme molecule.
10. The method of statement 1, wherein said TGFβ signaling inhibitor is a small molecule organic compound.
11. The method of statement 1, wherein said TGFβ signaling inhibitor antagonizes the activation of latent TGFβ to its active form capable of activating signaling via a TGFβ receptor.
12. The method of statement 1, wherein the HSCs are obtained from the bone marrow or blood (e.g., umbilical cord blood) of a human subject.
13. The method of statement 10, wherein the human subject suffers from a disease caused by a genetic defect that affects hematopoietic cells, for example, Fanconi Anemia (FA), sickle cell anemia, severe combined immunodeficiency, β-thalassemia, Wiskott-Aldrich syndrome, and leukodystrophy.
14. The method of statement 1, wherein the HSCs are expanded by 2 fold, 3 fold, 4 fold or more.
15. The method of statement 1, wherein the normoxic condition comprises an oxygen level of more than 3%.

### Method Statement Set 4

1. A method of maintaining hematopoietic stem cells (HSCs) in vitro comprising culturing the HSCs under hypoxic conditions.
2. The method of statement 1, wherein the hypoxic condition comprises an oxygen level of less than 3%.
3. The method of statement 1, wherein the HCSs are adult HSCs or umbilical cord blood HSCs.
4. The method of statement 1, wherein the HSCs are obtained from the bone marrow or blood of a human subject.
5. The method of statement 4, wherein the human subject suffers from a disease caused by a genetic defect that affects hematopoietic cells, for example, Fanconi Anemia (FA), sickle cell anemia, severe combined immunodeficiency, β-thalassemia, Wiskott-Aldrich syndrome, and leukodystrophy.

### Method Statement Set 5

1. A method of maintaining hematopoietic stem cells (HSCs) in vitro comprising culturing the HSCs under normoxic conditions and in a culturing medium that comprises a factor selected from the group consisting of a hypoxia mimetic, a ROS scavenger, and a HIF stabilizer.
2. The method of statement 1, wherein the hypoxia mimetic is desferrioxamine (DFO).
3. The method of statement 1, wherein the HSCs are cultured in a culturing medium that comprises a factor selected from the group consisting of a ROS scavenger, and a HIF stabilizer.
4. The method of statement 1, wherein the HIF stabilizer is selected from the group consisting of Roxadustat (RXD) (FG-4592), Vadadustat (AKB-6548), Daprodustat (GSK-1278863), and Molidustat (BAY 85-3934).
5. The method of statement 1, wherein the ROS scavenger is selected from the group consisting of N-acetyl-L-cysteine (NAC), Sodium pyruvate, N,N′-dimethylthiourea (DMTU), Trolox (6-hydroxy-2,5,7,8-tetramethylchroman-2-carboxylic acid), α-tocopherol, Ebselen (2-phenyl-1,2-benzisoselenazol-3(2H)-one), uric acid, Vitamin C, and MnTBAP (manganese(III)-tetrakis(4-benzoic acid)porphyrin).
6. The method of statement 1, wherein the HSCs are obtained from the bone marrow or blood (e.g., umbilical cord blood) of a human subject.
7. The method of statement 6, wherein the human subject suffers from a disease caused by a genetic defect that affects hematopoietic cells, for example, Fanconi Anemia (FA), sickle cell anemia, severe combined immunodeficiency, β-thalassemia, Wiskott-Aldrich syndrome, and leukodystrophy.

### Method Statement Set 6

1. A method of treating subject suffering from a disease comprising a genetic defect affecting hematopoietic cells comprising:
   harvesting hematopoietic stem cells (HSCs) from the subject,
   culturing the HSCs according to any one of the methods in Method Sets 1-5;
   genetically manipulating the expanded HSCs to correct the genetic defect; and
   administering the genetically-manipulated HSC to the subject.
2. The method of statement 1, wherein the disease is selected from the group consisting of Fanconi Anemia (FA), sickle cell anemia, severe combined immunodeficiency, β-thalassemia, Wiskott-Aldrich syndrome, and leukodystrophy.
3. The method of statement 1, wherein the HSCs are obtained from the bone marrow or blood (e.g., umbilical cord blood) of a human subject.

The specific methods, devices and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising expanding hematopoietic stem cells and/or hematopoietic progenitor cells ex vivo under conditions comprising reduced atmospheric oxygen levels or reduced reactive oxygen species (ROS) levels in the presence of endothelial cells, to thereby generate an expanded population of hematopoietic stem cells and/or hematopoietic progenitor cells.

2. The method of claim 1, wherein the reduced atmospheric oxygen levels comprise an atmosphere of 1% oxygen, or less than 1% oxygen.

3. The method of claim 1, wherein the conditions comprise a culture medium that supports the growth of the hematopoietic stem cells and/or hematopoietic progenitor cell, wherein the culture medium further comprises at least one cytokine.

4. The method of claim 1, wherein the reduced reactive oxygen species (ROS) levels comprise at least one reactive oxygen species (ROS) scavenger in a culture medium that supports the growth of the hematopoietic stem cells and/or hematopoietic progenitor cell.

5. (canceled)

6. The method of claim 1, wherein the culture medium further comprises heparin or comprises a TGFβ inhibitor, a TGFβ signaling inhibitor, a chemokine, a hypoxia mimetic, a ROS scavenger, a HIF stabilizer, an mTOR inhibitor, BMP4, or a combination thereof.

7. The method of claim 1, wherein the conditions comprise reduced reactive oxygen species (ROS) and a normoxic atmosphere.

8. (canceled)

9. The method of claim 1, wherein the endothelial cells are within microcarriers, optionally dissolvable microcarriers.

10. The method of claim 1, wherein the endothelial cells are not genetically modified human endothelial cells.

11. The method of claim 1, wherein the endothelial cells express an exogenous wild type or genetically modified adenovirus E4 open reading frame 1 (E4ORF1) polypeptide or express an Akt gene from an exogenous expression cassette or exogenous expression vector.

12. (canceled)

13. The method of claim 1, wherein the hematopoietic stem cells and/or hematopoietic progenitor cells are obtained from donor bone marrow, umbilical donor cord blood, or donor peripheral blood.

14. The method of claim 1, which expands the hematopoietic stem cells and/or hematopoietic progenitor cells by at least 10 fold, or at least 40 fold or wherein the expanded population of hematopoietic stem cells and/or hematopoietic progenitor cells is from a single cell or from less than 1000 cells and optionally wherein the single cell or the less than 1000 cells is expanded into a population of at least 40 million hematopoietic stem cells and/or hematopoietic progenitor cells.

15-16. (canceled)

17. The method of claim 1, wherein diseased or mutant hematopoietic stem cells and/or hematopoietic progenitor cells are expanded to the same extent as healthy or wild type hematopoietic stem cells and/or hematopoietic progenitor cells.

18. The method of claim 1, further comprising administering the expanded population of hematopoietic stem cells and/or hematopoietic progenitor cells to a subject and optionally wherein expanded population of hematopoietic stem cells and/or hematopoietic progenitor cells administered to the subject are an expanded population of autologous hematopoietic stem cells and/or autologous hematopoietic progenitor cells.

19. The method of claim 1, wherein the hematopoietic stem cells and/or hematopoietic progenitor cells are from a subject suffering from a disease or condition or wherein the subject suffers from a disease caused by a genetic defect that affects hematopoietic cells, a metabolic condition, an infection of hematopoietic cells, or a reaction to a drug.

20. (canceled)

21. The method of claim 1, further comprising genetically modifying the hematopoietic stem cells and/or hematopoietic progenitor cells to generate genetically modified hematopoietic stem cells and/or hematopoietic progenitor cells, and then expanding the genetically modified hematopoietic stem cells and/or hematopoietic progenitor cells and optionally wherein the genetically modifying the hematopoietic stem cells and/or hematopoietic progenitor cells obviates or alleviates the genetic defect that affects hematopoietic cells, the metabolic condition, the infection of hematopoietic cells, or the reaction to a drug.

22-23. (canceled)

24. The method of claim 1, further comprising aliquoting portions of the hematopoietic stem cells and/or hematopoietic progenitor cells into a series of separate receptacles, adding one or more different test agents to the separate receptacles to form a series of test mixtures, expanding the hematopoietic stem cells and/or hematopoietic progenitor cells in the test mixtures, and quantifying the types of cells within the test mixtures after expansion to obtain a series of quantified cell numbers for the different test mixtures and optionally further comprising comparing each of the series of quantified cell numbers with a cell number control or an average quantified control cell number, wherein optionally the hematopoietic stem cells and/or hematopoietic progenitor cells are from a donor or subject with a disease or condition or optionally wherein the disease or condition is cancer.

25-27. (canceled)

28. A composition comprising a population of hematopoietic stem cells and/or hematopoietic progenitor cells expanded by the method of claim 1 which composition optionally further comprises a TGFβ inhibitor of a TGFβ family member, a TGFβ signaling inhibitor, a chemokine, a hypoxia mimetic, a ROS scavenger, a HIF stabilizer, an mTOR inhibitor, BMP4, or a combination thereof.

29. A composition comprising a population of hematopoietic stem cells and/or hematopoietic progenitor cells in a solution comprising less than 1% molecular oxygen or comprising hematopoietic stem cells and/or hematopoietic progenitor cells and microcarrier-encapsulated endothelial cells, wherein the microcarriers are digestible by one or more enzymes, which composition optionally further comprises a TGFβ inhibitor of a TGFβ family member, a TGFβ signaling inhibitor, a chemokine, a hypoxia mimetic, a ROS scavenger, a HIF stabilizer, an mTOR inhibitor, BMP4, or a combination thereof.

30. (canceled)

31. The composition of claim 28, comprising at least 10⁸ cells.

32. The composition of claim 28, wherein the hematopoietic stem cells and/or hematopoietic progenitor cells are expanded from cells obtained from a single donor or person.

33. (canceled)
